SEQUENCE LISTING (3 sequences)

SEQ ID NO: 1 - DNA, 8833 bp, Artificial Sequence
  Synthetic prepro-VWF
agctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaagatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcaccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgaggctgctgcagctgcatgggtgcctgctgctgcctgccttggcctgatggccaggccagagtgctgccagtcctctgcatgttctgctcttgtgcccttctgagcccacaataaaggctgagctcttatcttgcaaaaggc

SEQ ID NO: 2 - protein, 2783 aa, Artificial Sequence
  Synthetic prepro-VWF
MIPARFAGVLLLILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKLETEAGYYKLSGEAYGFVARIDGSGNFQVLLSDRYFNKTCGLCGNFNIFAEDDFMTQEGTLTSDPYDFANSWALSSGEQWCERPSSSCNISSGEMQKGLWEQCQLLKSTSVFARCHPLVDPEPFCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGSYAAACAGRGVRVAWREPGRCELNCPKGQVYLQCGTPCNLTCRSLSYPDEECNEACLEGCFCPPMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQHCTNGSVVYHEVLNAMECKCSPRKCSK

SEQ ID NO: 3 - protein, 2050 aa, Artificial Sequence
  Synthetic mature VWF
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

(19) United States
(12) Patent Application Publication
(10) Pub. No.: US 2022/0395560 A1
(43) Pub. Date: Dec. 15, 2022

(54) METHODS OF PROPHYLACTIC TREATMENT USING RECOMBINANT VWF (rVWF)

(71) Applicant: Takeda Pharmaceutical Company Limited, Osaka (JP)

(72) Inventors: Björn Mellgård, Cambridge, MA (US); Bruce Ewenstein, Brookline, MA (US)

(21) Appl. No.: 17/362,191

(22) Filed: Jun. 29, 2021

Related U.S. Application Data
(63) Continuation-in-part of application No. 16/778,771, filed on Jan. 31, 2020.
(60) Provisional application No. 62/800,370, filed on Feb. 1, 2019.

(51) Int. Cl. A61K 38/36; A61K 38/37; A61P 7/04

(52) U.S. Cl. CPC A61K 38/36; A61P 7/04; A61K 38/37

(57) ABSTRACT

The present invention relates to a method for prophylactic treatment of spontaneous bleeding in a subject with severe von Willebrand Disease comprising administering a therapeutic amount of recombinant von Willebrand Factor (rVWF) to the subject.

### CROSS REFERENCE TO RELATED APPLICATION

This application is a continuation-in-part of U.S. application Ser. No. 16/778,771, filed on Jan. 31, 2020, which claims priority to U.S. Provisional Patent Application No. 62/800,370, filed on Feb. 1, 2019, which are hereby incorporated by reference in their entireties.

### REFERENCE TO SEQUENCE LISTING

The Sequence Listing text copy submitted herewith via EFS-Web was created on Mar. 30, 2020, is entitled 008073-5207-US_Sequence_Listing.txt, is 53,949 bytes in size and is herein incorporated by reference in its entirety.

### BACKGROUND OF THE INVENTION

Coagulation diseases, such as von Willebrand Disease (VWD) generally result from a deficiency in the coagulation cascade. von Willebrand Disease (VWD) refers to the group of diseases caused by a deficiency of von Willebrand factor. Von Willebrand factor helps blood platelets clump together and stick to the blood vessel wall, which is necessary for normal blood clotting.

von Willebrand disease (VWD) is the most common inherited bleeding disorder, with an estimated prevalence rate of 1% (Veyradier A, et al., Medicine (Baltimore). 2016, 95(11):e3038). However, excluding milder forms of the disease, only about 1/10,000 patients actually require treatment. Current treatment for these coagulopathies includes a replacement therapy using pharmaceutical preparations comprising the normal coagulation factor.

VWF is a glycoprotein circulating in plasma as a series of multimers ranging in size from about 500 to 20,000 kD. The full length of cDNA of VWF has been cloned; the propolypeptide corresponds to amino acid residues 23 to 764 of the full-length prepro-VWF (Eikenboom et al (1995) Haemophilia 1, 77 90). Multimeric forms of VWF are composed of 250 kD polypeptide subunits linked together by disulfide bonds. VWF mediates the initial platelet adhesion to the sub-endothelium of the damaged vessel wall, with the larger multimers exhibiting enhanced hemostatic activity. Multimerized VWF binds to the platelet surface glycoprotein Gp1bα, through an interaction in the A1 domain of VWF, facilitating platelet adhesion. Other sites on VWF mediate binding to the blood vessel wall. Thus, VWF forms a bridge between the platelet and the vessel wall that is essential to platelet adhesion and primary hemostasis under conditions of high shear stress. Normally, endothelial cells secrete large polymeric forms of VWF and those forms of VWF that have a lower molecular weight arise from proteolytic cleavage. The multimers of exceptionally large molecular masses are stored in the Weibel-Pallade bodies of the endothelial cells and liberated upon stimulation by agonists such as thrombin and histamine.

For patients with VWD, it is recommended that they be treated with von Willebrand factor (VWF) replacement given the need for prolonged hemostasis, particularly in major surgery and in response to other bleeding episodes. However, prophylactic treatments are not well described or known. There remains a need in the art to help patients in advance of such bleeding episodes by means of prophylactic treatment regimens. The present invention meets this need by providing methods for the prophylactic treatment of VWD with human rVWF.

### BRIEF SUMMARY OF THE INVENTION

The present invention provides a method for prophylactically treating spontaneous bleeding episodes in a subject with severe von Willebrand Disease (VWD). The method comprises administering to the subject twice-weekly at least one dose of recombinant von Willebrand Factor (rVWF) ranging from at least about 40 IU/kg to about 80 IU/kg, thereby reducing the frequency and/or duration of spontaneous bleeding episodes.

In some embodiments, the at least one dose of rVWF ranges from at least about 50 IU/kg to about 80 IU/kg.

In some embodiments, the subject has a baseline VWF ristocetin cofactor activity (VWF:RCo) ranging from 20 IU/dL or less, or is diagnosed with Type 1 VWD. In certain embodiments, the subject is diagnosed with Type 2A, 2B, or 2M VWD. In particular embodiments, the subject has a VWF:antigen content (VWF:Ag) ranging from 3 IU/dL or greater, or is diagnosed with Type 3 VWD.

In some embodiments, the subject has been administered prophylactic treatment of plasma-derived VWF (pdVWF) within the past 12 months before initial administration of rVWF.

In some embodiments, the subject has experienced at least 3 spontaneous bleeding episodes within the past 12 months.

In some embodiments of the method, the administration of rVWF occurs every 3 to 4 days. In some embodiments, the administration occurs on day 1 and day 5, day 2 and day 6, or day 3 and day 7 of a 7 day period. In certain embodiments, the administration occurs at least every 24 hours, 36 hours, 48 hours, 72 hours, or 84 hours. In some embodiments, the administration occurs at least every 72 hours.

In some embodiments, the method as outlined further comprises administering to the subject at least one dose of recombinant Factor VIII (rFVIII). In some embodiments, the administration of the at least one dose of rFVIII is concomitantly or sequentially administered with the at least one dose of rVWF.

In some embodiments, the subject resumes the prophylactic treatment after receiving elective surgery or oral surgery. In some embodiments, if the elective surgery is minor surgery or oral surgery and the subject has FVIII activity (FVIII:C) of at least 0.4 IU/mL or greater, the subject is administered rVWF without rFVIII before surgery. In certain embodiments, if the elective surgery is major surgery and the subject has FVIII activity (FVIII:C) of at least 0.8 IU/mL or greater, the subject is administered rVWF without rFVIII before surgery.

In some embodiments, prophylactic treatment efficacy is indicated by a reduction of ≥25% in annual bleeding rate (ABR) for spontaneous bleeding episodes during rVWF prophylaxis relative to the pretreatment ABR.

In some embodiments, prophylactic treatment efficacy is indicated by a reduction of ≥25%, ≥30%, ≥35%, ≥40%, ≥45%, ≥50%, ≥55%, ≥60%, ≥65%, ≥70%, ≥75%, ≥80%, ≥85%, ≥90%, or ≥95% in annual bleeding rate (ABR) for spontaneous bleeding episodes during rVWF prophylaxis relative to the pretreatment ABR.

In some embodiments, prophylactic treatment efficacy is measured by examining vWF:RCo and/or FVIII activities in samples obtained from the subject before and after prophylactic treatment with rVWF.

In some embodiments, prophylactic treatment efficacy is measured by examining FVIII, FVIII:C, VWF:RCo, VWF:Ag, and/or VWF collagen-binding capacity activities in samples obtained from the subject before and after prophylactic treatment with rVWF.

In some embodiments, the samples for examining FVIII, FVIII:C, VWF:RCo, VWF:Ag, and/or VWF collagen-binding capacity activities are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF.

In some embodiments, the samples for examining FVIII, FVIII:C, VWF:RCo, VWF:Ag, and/or VWF collagen-binding capacity activities are obtained 25 to 31 days after prophylactic treatment with rVWF.

In some embodiments, prophylactic treatment efficacy is determined after or during a bleeding episode, wherein samples for examining FVIII, FVIII:C, VWF:RCo, VWF:Ag, and/or VWF collagen-binding capacity activities are obtained after the bleeding episode, and further wherein the samples are obtained prior to rVWF administration, 2 hours after rVWF administration and then every 12-24 hours until resolution of the bleeding episode.

In some embodiments, prophylactic treatment efficacy is indicated by an improvement in FVIII, FVIII:C, VWF:RCo, VWF:Ag, and/or VWF collagen-binding capacity activity levels after the prophylactic treatment with rVWF as compared to the levels prior to the prophylactic treatment with rVWF.

Also provided herein is a method for prophylactically treating spontaneous bleeding episodes in a subject with severe von Willebrand Disease (VWD). The method comprises administering to the subject a weekly dose of recombinant von Willebrand Factor (rVWF) substantially equivalent to a corresponding weekly dose of plasma-derived VWF (pdVWF) previously administered to said subject, thereby reducing the frequency and/or duration of spontaneous bleeding episodes.

In some embodiments, the weekly dose of rVWF is about 10% less than the corresponding weekly dose of pdVWF. In some embodiments, the weekly dose of rVWF is about 10% more than the corresponding weekly dose of pdVWF.

In some embodiments, the weekly dose of rVWF is two individual infusions administered on separate days. In some embodiments, the weekly dose of rVWF is three individual infusions administered on separate days. In some embodiments, the weekly dose of rVWF is a single infusion.

In some embodiments, each individual infusion comprises up to 80 IU/kg rVWF.

In some embodiments, each individual infusion comprises 80 IU/kg rVWF.

In some embodiments, each individual infusion comprises 50 IU/kg rVWF.

In some embodiments, the subject has a baseline VWF ristocetin cofactor activity (VWF:RCo) ranging from 20 IU/dL or less, or is diagnosed with Type 1 VWD. In some embodiments, the subject is diagnosed with Type 2A, 2B, or 2M VWD. In some embodiments, the subject has a VWF:antigen content (VWF:Ag) ranging from 3 IU/dL or greater, or is diagnosed with Type 3 VWD.

In some embodiments, the subject has received prophylactic treatment of pdVWF for at least 12 months.

In some embodiments, the two individual infusions are administered on day 1 and day 5, or day 2 and day 6, or day 3 and day 7 of a 7 day period. In certain embodiments, the three individual infusions are administered on day 1, day 3, and day 6 of a 7 day period. In some embodiments, the administration occurs at least every 24 hours, 36 hours, 48 hours, 72 hours, or 84 hours. In particular embodiments, the administration occurs at least every 72 hours.

In some embodiments, the method provided herein further comprises administering to the subject at least one dose of recombinant Factor VIII (rFVIII).

In some embodiments, the administration of the at least one dose of rFVIII is concomitantly or sequentially administered with the weekly dose of rVWF.

In some embodiments, the subject resumes the prophylactic treatment after receiving elective surgery or oral surgery. In some embodiments, if the elective surgery is minor surgery or oral surgery and the subject has a FVIII activity (FVIII:C) of at least 0.4 IU/mL or greater, the subject is administered rVWF without rFVIII prior to surgery. In some embodiments, if the elective surgery is major surgery and the subject has a FVIII activity (FVIII:C) of at least 0.8 IU/mL or greater, the subject is administered rVWF without rFVIII prior to surgery.

In some embodiments, prophylactic treatment efficacy is indicated by a reduction of ≥25% in annual bleeding rate (ABR) for spontaneous bleeding episodes during rVWF prophylaxis relative to the pretreatment ABR.

In some embodiments, prophylactic treatment efficacy is indicated by a reduction of ≥25%, ≥30%, ≥35%, ≥40%, ≥45%, ≥50%, ≥55%, ≥60%, ≥65%, ≥70%, ≥75%, ≥80%, ≥85%, ≥90%, or ≥95% in annual bleeding rate (ABR) for spontaneous bleeding episodes during rVWF prophylaxis relative to the pretreatment ABR.

In some embodiments, prophylactic treatment efficacy is measured by examining vWF:RCo and/or FVIII activities in samples obtained from the subject before and after prophylactic treatment with rVWF.

In some embodiments, prophylactic treatment efficacy is measured by examining FVIII, FVIII:C, VWF:RCo, VWF:Ag, and/or VWF collagen-binding capacity activities in samples obtained from the subject before and after prophylactic treatment with rVWF.

In some embodiments, the samples for examining FVIII, FVIII:C, VWF:RCo, VWF:Ag, and/or VWF collagen-binding capacity activities are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF.

In some embodiments, the samples for examining FVIII, FVIII:C, VWF:RCo, VWF:Ag, and/or VWF collagen-binding capacity activities are obtained 25 to 31 days after prophylactic treatment with rVWF.

In some embodiments, prophylactic treatment efficacy is determined after or during a bleeding episode, wherein samples for examining FVIII, FVIII:C, VWF:RCo, VWF:Ag, and/or VWF collagen-binding capacity activities are obtained after the bleeding episode, and further wherein the samples are obtained prior to rVWF administration, 2 hours after rVWF administration and then every 12-24 hours until resolution of the bleeding episode.

In some embodiments, prophylactic treatment efficacy is indicated by an improvement in FVIII, FVIII:C, VWF:RCo, VWF:Ag, and/or VWF collagen-binding capacity activity levels after the prophylactic treatment with rVWF as compared to the levels prior to the prophylactic treatment with rVWF.

In some embodiments, spontaneous bleeding or bleeding episode comprises any one selected from the group consisting of hemarthrosis, epistaxis, muscle bleeding, oral bleeding, and gastrointestinal bleeding.

In some embodiments of any of the methods described, the subject is not diagnosed with type 2N VWD or pseudo VWD.

Other objects, advantages and embodiments of the invention will be apparent from the detailed description following.

### BRIEF DESCRIPTION OF THE DRAWINGS

FIG. 1 shows the study design for the global, multicenter, open-label, phase 3 study (NCT02973087, EudraCT no.: 2016-001478-14) described herein.

FIG. 2 shows a table of the key study inclusion and exclusion criteria.

FIG. 3 shows a table of exemplary dosing schedules for rVWF prophylaxis.

FIG. 4 shows a table of main study outcome measures.

FIG. 5A-FIG. 5C depict the nucleic acid sequence of human recombinant prepro-VWF.

FIG. 6A-FIG. 6J depict the amino acid sequence of human recombinant prepro-VWF.

FIG. 7A-FIG. 7G depict the amino acid sequence of human recombinant mature VWF.

### DETAILED DESCRIPTION OF THE INVENTION

### Introduction

For patients with VWD, it is recommended that they be treated with von Willebrand factor (VWF) replacement given the need for prolonged hemostasis, particularly in major surgery (Mannucci P M and Franchini M., Haemophilia, 2017, 23(2):182-187; National Institutes of Health. National Heart, Lung, and Blood Institute. The Diagnosis, Evaluation, and Management of von Willebrand Disease NIH Publication No. 08-5832; December, 2007). Plasma-derived VWF therapies contain factor VIII (FVIII) and have the potential for FVIII accumulation with repeated dosing.

VONVENDI® which is approved for the US and its EU approved equivalent, VEYVONDI® each cover recombinant von Willebrand factor (rVWF) concentrates that are manufactured by recombinant DNA technology in the Chinese hamster ovary cell line without the addition of exogenous human or animal-derived protein (Turecek P L, et al. Hamostaseologie. 2009; 29(suppl 1):S32-38; Mannucci P M, et al. Blood, 2013; 122(5):648-657; Gill J C, et al. Blood, 2015; 126(17):2038-2046; European Medicines Agency. VEYVONDI Summary of Product Characteristics). rVWF contains the full VWF multimer profile, including ultra-large multimers that are usually deficient in plasma-derived VWF (pdVWF) concentrates exposed to ADAMTS13, the VWF-cleaving protein.

Most patient with VWD present with mild to moderate mucosal bleeding and bleeding after trauma or surgery, although life-threatening bleeding may also occur, particularly in patient with severe disease (Nichols et al., Haemophilia, 2008, 14:171-232; Leebeek F W and Eikenboom J C, N Engl J Med, 2016, 375:2067-80). Reducing the frequency and duration of bleeding episodes would be expected to decrease the need for red blood cell transfusions and lower the risk of debilitating comorbidities including arthopathy.

Patients with severe VWD may benefit from prophylactic rVWF treatment to maintain VWF and FVIII levels so that the risk of spontaneous bleeding episodes (BEs), including hemarthrosis, epistaxis, and gastrointestinal bleeding, is reduced (Abshire T C, Thromb Res 2009, 124(suppl 1):523-6; Berntorp E, Haemophilia 2008, 14(suppl 5):47-53; Berntorp E and Petrini P, Blood Coagul Fibrinolysis, 2005, 16 (suppl 1): S23-6; Berntorp E, Semin Thromb Hemost, 2006, 32:621-5; Abshire et al., Haemophilia, 2013, 19:76-81; Abshire TC, J Thromb Haemost, 2015, 13:1585-9).

The present invention provides methods for prophylactically treating spontaneous bleeding in a patient with severe von Willebrand Disease (VWD). The method comprises administering to the subject recombinant von Willebrand factor (rVWF). In some embodiments, the subject is administered a twice-weekly dose ranging from about 40-80 IU/kg of rVWF. In some embodiments, the subject is administered a weekly dose ranging from about 40-80 IU/kg of rVWF. In some instances, the weekly dose is provided as a single administration once a week. In certain instances, the weekly dose is provided as two separate administrations on different days over a week. In other instances, the weekly dose is provided as three separate administrations on different days over a week.

The disclosure of PCT Application Publication No. WO2012/171031 is herein incorporated by reference in its entirety for all purposes.

### Definitions

Before the invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

As used herein. “rVWF” refers to recombinant VWF.

As used herein, “rFVIII” refers to recombinant FVIII.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, “recombinant VWF” includes VWF obtained via recombinant DNA technology. In certain embodiments, VWF proteins of the invention can comprise a construct, for example, prepared as in WO 1986/06096 published on Oct. 23, 1986 and U.S. patent application Ser. No. 07/559,509, filed on Jul. 23, 1990, in the name of Ginsburg et al., which is incorporated herein by reference with respect to the methods of producing recombinant VWF. The VWF in the present invention can include all potential forms, including the monomeric and multimeric forms. It should also be understood that the present invention encompasses different forms of VWF to be used in combination. For example, the VWF of the present invention may include different multimers, different derivatives and both biologically active derivatives and derivatives not biologically active.

In the context of the present invention, the recombinant VWF embraces any member of the VWF family from, for example, a mammal such as a primate, human, monkey, rabbit, pig, rodent, mouse, rat, hamster, gerbil, canine, feline, and biologically active derivatives thereof. Mutant and variant VWF proteins having activity are also embraced, as are functional fragments and fusion proteins of the VWF proteins. Furthermore, the VWF of the invention may further comprise tags that facilitate purification, detection, or both. The VWF described herein may further be modified with a therapeutic moiety or a moiety suitable imaging in vitro or in vivo.

As used herein, “plasma-derived VWF” or “pdVWF” includes all forms of the protein found in blood including the mature VWF obtained from a mammal having the property of in vivo-stabilizing, e.g. binding, of at least one FVIII molecule.

The term “highly multimeric VWF” or “high molecular weight VWF” refers to VWF comprising at least 10 subunits, or 12, 14, or 16 subunits, to about 20, 22, 24 or 26 subunits or more. The term “subunit” refers to a monomer of VWF. As is known in the art, it is generally dimers of VWF that polymerize to form the larger order multimers (see Turecek et al., Semin. Thromb. Hemost. 2010, 36(5): 510-521 which is hereby incorporated by reference in its entirety for all purposes and in particular for all teachings regarding multimer analysis of VWF).

As used herein, the term “factor VIII” or “FVIII” refers to any form of factor VIII molecule with the typical characteristics of blood coagulation factor VIII, whether endogenous to a patient, derived from blood plasma, or produced through the use of recombinant DNA techniques, and including all modified forms of factor VIII. Factor VIII (FVIII) exists naturally and in therapeutic preparations as a heterogeneous distribution of polypeptides arising from a single gene product (see, e.g., Andersson et al., Proc. Natl. Acad. Sci. USA, 83:2979-2983 (1986)). Commercially available examples of therapeutic preparations containing Factor VIII include those sold under the trade names of HEMOFIL M, ADVATE, and RECOMBINATE (available from Baxter Healthcare Corporation, Deerfield, Ill., U.S.A.).

As used herein, “plasma FVIII activity” and “in vivo FVIII activity” are used interchangeably. The in vivo FVIII activity measured using standard assays may be endogenous FVIII activity, the activity of a therapeutically administered FVIII (recombinant or plasma derived), or both endogenous and administered FVIII activity. Similarly, “plasma FVIII” refers to endogenous FVIII or administered recombinant or plasma derived FVIII.

As used herein “von Willebrand Disease” refers to the group of diseases caused by a deficiency of von Willebrand factor. Von Willebrand factor helps blood platelets clump together and stick to the blood vessel wall, which is necessary for normal blood clotting. As described in further detail herein, there are several types of Von Willebrand disease including type 1, 2A, 2B, 2M and 3.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. VWF is the predominant species present in a preparation is substantially purified. The term “purified” in some embodiments denotes that a nucleic acid or protein gives rise to essentially one band in an electrophoretic gel. In other embodiments, it means that the nucleic acid or protein is at least 50% pure, more preferably at least 60%, 65%, 70%, 75%, 80%, 85%, 90%, 95%, 96%, 97%, 98%, 99% or more pure. “Purify” or “purification” in other embodiments means removing at least one contaminant from the composition to be purified. In this sense, purification does not require that the purified compound be homogenous, e.g., 100% pure.

As used herein, “administering” (and all grammatical equivalents) includes intravenous administration, intramuscular administration, subcutaneous administration, oral administration, administration as a suppository, topical contact, intraperitoneal, intralesional, or intranasal administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “therapeutically effective amount or dose” or “therapeutically sufficient amount or dose” or “effective or sufficient amount or dose” refer to a dose that produces therapeutic effects for which it is administered. For example, a therapeutically effective amount of a drug useful for treating hemophilia can be the amount that is capable of preventing or relieving one or more symptoms associated with hemophilia. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the terms “patient” and “subject” are used interchangeably and refer to a mammal (preferably human) that has a disease or has the potential of contracting a disease.

As used herein, the term “about” denotes an approximate range of plus or minus 10% from a specified value. For instance, the language “about 20%” encompasses a range of 18-22%.

As used herein, the term “half-life” refers to the period of time it takes for the amount of a substance undergoing decay (or clearance from a sample or from a patient) to decrease by half.

I. Recombinant von Willebrand Factor (rVWF)

The present invention utilizes compositions comprising von Willebrand Factor (rVWF) for prophylactic treatment for spontaneous bleeding episodes in a subject with severe VWD. In some embodiments, the treatment reduces the severity, incidence (frequency) and/or duration of spontaneous bleeding episodes.

In certain embodiments, VWF proteins of the invention may comprise a construct, for example, prepared as in WO 1986/06096 published on Oct. 23, 1986 and U.S. patent application Ser. No. 07/559,509, filed on Jul. 23, 1990, in the name of Ginsburg et al., which is incorporated herein by reference with respect to the methods of producing recombinant VWF. The VWF useful for the present invention includes all potential forms, including the monomeric and multimeric forms. One particularly useful form of VWF are homo-multimers of at least two VWFs. The VWF proteins may be either a biologically active derivative, or when to be used solely as a stabilizer for FVIII the VWF may be of a form not biologically active. It should also be understood that the present invention encompasses different forms of VWF to be used in combination. For example, a composition useful for the present invention may include different multimers, different derivatives and both biologically active derivatives and derivatives not biologically active.

In primary hemostasis VWF serves as a bridge between platelets and specific components of the extracellular matrix, such as collagen. The biological activity of VWF in this process can be measured by different in vitro assays (Turecek et al., Semin. Thromb. Hemost. 28: 149-160, 2002). The ristocetin cofactor assay is based on the agglutination of fresh or formalin-fixed platelets induced by the antibiotic ristocetin in the presence of VWF.

The degree of platelet agglutination depends on the VWF concentration and can be measured by the turbidimetric method, e.g. by use of an aggregometer (Weiss et al., J. Clin. Invest. 52: 2708-2716, 1973; Macfarlane et al., Thromb. Diath. Haemorrh. 34: 306-308, 1975). The second method is the collagen binding assay, which is based on ELISA technology (Brown et Bosak, Thromb. Res. 43: 303-311, 1986; Favaloro, Thromb. Haemost. 83: 127-135, 2000). A microtiter plate is coated with type I or III collagen. Then the VWF is bound to the collagen surface and subsequently detected with an enzyme-labeled polyclonal antibody. The last step is the substrate reaction, which can be photometrically monitored with an ELISA reader. As provided herein, the specific Ristocetin Cofactor activity of the VWF (VWF:RCo) of the present invention is generally described in terms of mU/μg of VWF, as measured using in vitro assays.

An advantage of the rVWF compositions of the present invention over pdVWF is that rVWF exhibits a higher specific activity than pdVWF. In some embodiments, the rVWF of the invention has a specific activity of at least about 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 105, 110, 115, 120, 125, 130, 135, 140, 145, 150 or more mU/μg.

The rVWF of the present invention is highly multimeric comprising about 10 to about 40 subunits. In further embodiments, the multimeric rVWF produced using methods of the present invention comprise about 10-30, 12-28, 14-26, 16-24, 18-22, 20-21 subunits. In further embodiments, the rVWF is present in multimers varying in size from dimers to multimers of over 40 subunits (>10 million Daltons). The largest multimers provide multiple binding sites that can interact with both platelet receptors and subendothelial matrix sites of injury, and are the most hemostatically active form of VWF. Application of ADAMTS13 will cleave the ultra-large rVWF multimers over time, but during production (generally through expression in cell culture), rVWF compositions of the present invention are generally not exposed to ADAMTS13 and retain their highly multimeric structure.

In one embodiment, a rVWF composition used in the methods described herein has a distribution of rVWF oligomers characterized in that 95% of the oligomers have between 6 subunits and 20 subunits. In other embodiments, the a rVWF composition has a distribution of rVWF oligomers characterized in that 95% of the oligomers have a range of subunits selected from variations 458 to 641 found in Table 2 of WO 2012/171031, which is herein incorporated by reference in its entirety for all purposes.

In one embodiment, a rVWF composition can be characterized according to the percentage of rVWF molecules that are present in a particular higher order rVWF multimer or larger multimer. For example, in one embodiment, at least 20% of rVWF molecules in a rVWF composition used in the methods described herein are present in an oligomeric complex of at least 10 subunits. In another embodiment, at least 20% of rVWF molecules in a rVWF composition used in the methods described herein are present in an oligomeric complex of at least 12 subunits. In yet other embodiments, a rVWF composition used in the methods provided herein has a minimal percentage (e.g., has at least X %) of rVWF molecules present in a particular higher-order rVWF multimer or larger multimer (e.g., a multimer of at least Y subunits) according to any one of variations 134 to 457 found in Table 3 to Table 5, which is herein incorporated by reference in its entirety for all purposes.

In accordance with the above, the rVWF composition administered to the subject (with or without FVIII) generally comprises a significant percentage of high molecular weight (HMW) rVWF multimers. In further embodiments, the HMW rVWF multimer composition comprises at least 10%-80% rVWF decamers or higher order multimers. In further embodiments, the composition comprises about 10-95%, 20-90%, 30-85%, 40-80%, 50-75%, 60-70% decamers or higher order multimers. In further embodiments, the BMW rVWF multimer composition comprises at least about 10%, 20%, 30%, 40%, 50%, 60%, 70%, 80%, 90% decamers or higher order multimers.

Assessment of the number and percentage of rVWF multimers can be conducted using methods known in the art, including without limitation methods using electrophoresis and size exclusion chromatography methods to separate VWF multimers by size, for example as discussed by Cumming et al., (J Clin Pathol. 1993 May; 46(5): 470-473, which is hereby incorporated by reference in its entirety for all purposes and in particular for all teachings related to assessment of VWF multimers). Such techniques may further include immunoblotting techniques (such as Western Blot), in which the gel is immunoblotted with a radiolabeled antibody against VWF followed by chemiluminescent detection (see, for example Wen et al., (1993), J. Clin. Lab. Anal., 7: 317-323, which is hereby incorporated by reference in its entirety for all purposes and in particular for all teachings related to assessment of VWF multimers). Further assays for VWF include VWF:Antigen (VWF:Ag), VWF:Ristocetin Cofactor (VWF:RCof), and VWF:Collagen Binding Activity assay (VWF:CBA), which are often used for diagnosis and classification of Von Willebrand Disease. (see for example Favaloro et al., Pathology, 1997, 29(4): 341-456, which is hereby incorporated by reference in its entirety for all purposes and in particular for all teachings related to assays for VWF).

In further embodiments, higher order rVWF multimers of the invention are stable for about 1 to about 90 hours post-administration. In still further embodiments, the higher order rVWF multimers are stable for about 5-80, 10-70, 15-60, 20-50, 25-40, 30-35 hours post-administration. In yet further embodiments, the higher order rVWF multimers are stable for at least 3, 6, 12, 18, 24, 36, 48, 72 hours post-administration. In certain embodiments the stability of the rVWF multimers is assessed in vitro.

In one embodiment, higher order rVWF multimers used in the compositions and methods provided herein have a half-life of at least 12 hours post administration. In another embodiment, the higher order rVWF multimers have a half-life of at least 24 hours post administration. In yet other embodiments, the higher order rVWF multimers have a half-life selected from variations 642 to 1045 found in Table 6 of WO 2012/171031, which is herein incorporated by reference in its entirety for all purposes.

In specific aspects, the rVWF (recombinant or plasma derived) used in accordance with the present invention are not modified with any conjugation, post-translation or covalent modifications. In particular embodiments, the rVWF of the present invention is not modified with a water soluble polymer, including without limitation, a polyethylene glycol (PEG), a polypropylene glycol, a polyoxyalkylene, a polysialic acid, hydroxyl ethyl starch, a poly-carbohydrate moiety, and the like.

In other aspects, the rVWF (recombinant or plasma derived) used in accordance with the present invention is modified through conjugation, post-translation modification, or covalent modification, including modifications of the N- or C-terminal residues as well as modifications of selected side chains, for example, at free sulfhydryl-groups, primary amines, and hydroxyl-groups. In one embodiment, a water soluble polymer is linked to the protein (directly or via a linker) by a lysine group or other primary amine. In one embodiment, the rVWF proteins of the present invention may be modified by conjugation of a water soluble polymer, including without limitation, a polyethylene glycol (PEG), a polypropylene glycol, a polyoxyalkylene, a polysialic acid, hydroxyl ethyl starch, a poly-carbohydrate moiety, and the like.

Water soluble polymers that may be used to modify the rVWF and/or FVIII include linear and branched structures. The conjugated polymers may be attached directly to the coagulation proteins of the invention, or alternatively may be attached through a linking moiety. Non-limiting examples of protein conjugation with water soluble polymers can be found in U.S. Pat. Nos. 4,640,835; 4,496,689; 4,301,144; 4,670,417; 4,791,192, and 4,179,337, as well as in Abuchowski and Davis “Enzymes as Drugs,” Holcenberg and Roberts, Eds., pp. 367 383, John Wiley and Sons, New York (1981), and Hermanson G., Bioconjugate Techniques 2nd Ed., Academic Press, Inc. 2008.

Protein conjugation may be performed by a number of well-known techniques in the art, for example, see Hermanson G., Bioconjugate Techniques 2nd Ed., Academic Press, Inc. 2008. Examples include linkage through the peptide bond between a carboxyl group on one of either the coagulation protein or water-soluble polymer moiety and an amine group of the other, or an ester linkage between a carboxyl group of one and a hydroxyl group of the other. Another linkage by which a coagulation protein of the invention could be conjugated to a water-soluble polymer compound is via a Schiff base, between a free amino group on the polymer moiety being reacted with an aldehyde group formed at the non-reducing end of the polymer by periodate oxidation (Jennings and Lugowski, J. Immunol. 1981; 127:1011-8; Femandes and Gregonradis, Biochim Biophys Acta. 1997; 1341; 26-34). The generated Schiff Base can be stabilized by specific reduction with NaCNBH₃ to form a secondary amine. An alternative approach is the generation of terminal free amino groups on the polymer by reductive amination with NH₄Cl after prior oxidation. Bifunctional reagents can be used for linking two amino or two hydroxyl groups. For example, a polymer containing an amino group can be coupled to an amino group of the coagulation protein with reagents like BS3 (Bis(sulfosuccinimidyl)suberate/Pierce, Rockford, Ill.). In addition, heterobifunctional cross linking reagents like Sulfo-EMCS (N-epsilon-Maleimidocaproyloxy) sulfosuccinimide ester/Pierce) can be used for instance to link amine and thiol groups. In other embodiments, an aldehyde reactive group, such as PEG alkoxide plus diethyl acetal of bromoacetaldehyde; PEG plus DMSO and acetic anhydride, and PEG chloride plus the phenoxide of 4-hydroxybenzaldehyde, succinimidyl active esters, activated dithiocarbonate PEG, 2,4,5-trichlorophenylcloroformate and P-nitrophenylcloroformate activated PEG, may be used in the conjugation of a coagulation protein.

In some aspects, the rVWF used in methods of the present invention has been matured in vitro with furin. In further embodiments, the furin is recombinant furin.

In further aspects, the rVWF used in the methods of the present invention are produced by expression in a mammalian cell culture using methods known in the art. In particular embodiments, the mammalian culture comprises CHO cells. In an exemplary embodiment, the rVWF of the invention comprises rVWF protein isolated from a CHO cell expression system. In a further embodiment, the propeptide removal is mediated in vitro through exposure of the pro-VWF to furin—in a still further embodiment, the Furin used for propeptide removal is recombinant furin. In as yet further embodiment, fully glycosylated/ABO blood group glycans are absent.

In yet further embodiments, the rVWF used in methods and compositions of the present invention by expression in a suitable eukaryotic host system. Examples of eukaryotic cells include, without limitation, mammalian cells, such as CHO, COS, HEK 293, BHK, SK-Hep, and HepG2; insect cells, e.g., SF9 cells, SF21 cells, S2 cells, and High Five cells; and yeast cells, e.g., Saccharomyces or Schizosaccharomyces cells. In one embodiment, the VWF can be expressed in yeast cells, insect cells, avian cells, mammalian cells, and the like. For example, in a human cell line, a hamster cell line, or a murine cell line. In one particular embodiment, the cell line is a CHO, BHK, or HEK cell line. Typically, mammalian cells, e.g., CHO cell from a continuous cell line, can be used to express the VWF of the present invention.

In certain embodiments, the nucleic acid sequence comprising a sequence coding for VWF can be a vector. The vector can be delivered by a virus or can be a plasmid. The nucleic acid sequence coding for the protein can be a specific gene or a biologically functional part thereof. In one embodiment, the protein is at least a biologically active part of VWF. A wide variety of vectors can be used for the expression of the VWF and can be selected from eukaryotic expression vectors. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

In some embodiments of the present invention, the nucleic acid sequence further comprises other sequences suitable for a controlled expression of a protein such as promoter sequences, enhancers, TATA boxes, transcription initiation sites, polylinkers, restriction sites, poly-A-sequences, protein processing sequences, selection markers, and the like which are generally known to a person of ordinary skill in the art.

In certain embodiments, the cell-culture methods of the invention may comprise the use of a microcarrier. In some embodiments, the cell-cultures of the embodiments can be performed in large bioreactors under conditions suitable for providing high volume-specific culture surface areas to achieve high cell densities and protein expression. One means for providing such growth conditions is to use microcarriers for cell-culture in stirred tank bioreactors. The concept of cell-growth on microcarriers was first described by van Wezel (van Wezel, A. L., Nature 216:64-5 (1967)) and allows for cell attachment on the surface of small solid particles suspended in the growth medium. These methods provide for high surface-to-volume ratios and thus allow for efficient nutrient utilization. Furthermore, for expression of secreted proteins in eukaryotic cell lines, the increased surface-to-volume ratio allows for higher levels of secretion and thus higher protein yields in the supernatant of the culture. Finally, these methods allow for the easy scale-up of eukaryotic expression cultures.

The cells expressing VWF can be bound to a spherical or a porous microcarrier during cell culture growth. The microcarrier can be a microcarrier selected from the group of microcarriers based on dextran, collagen, plastic, gelatine and cellulose and others as described in Butler (1988. In: Spier & Griffiths, Animal Cell Biotechnology 3:283-303). It is also possible to grow the cells to a biomass on spherical microcarriers and subculture the cells when they have reached final fermenter biomass and prior to production of the expressed protein on a porous microcarrier or vice versa. Suitable spherical microcarriers can include smooth surface microcarriers, such as Cytodex™ 1, Cytodex™ 2, and Cytode™ 3 (GE Healthcare) and macroporous microcarriers such as Cytopore™. 1, Cytopore™ 2, Cytoline™ 1, and Cytoline™ 2 (GE Healthcare).

In certain embodiments, rVWF is expressed in cells cultured in cell culture media that produces high molecular weight rVWF. The terms “cell culture solution,” “cell culture medium or media,” and “cell culture supernatant” refer to aspects of cell culture processes generally well known in the art. In the context of the present invention, a cell culture solution can include cell culture media and cell culture supernatant. The cell culture media are externally added to the cell culture solution, optionally together with supplements, to provide nutrients and other components for culturing the cells expressing VWF. The cell culture supernatant refers to a cell culture solution comprising the nutrients and other components from the cell culture medium as well as products released, metabolized, and/or excreted from the cells during culture. In further embodiments, the media can be animal protein-free and chemically defined. Methods of preparing animal protein-free and chemically defined culture media are known in the art, for example in US 2008/0009040 and US 2007/0212770, which are both incorporated herein for all purposes and in particular for all teachings related to cell culture media. “Protein free” and related terms refers to protein that is from a source exogenous to or other than the cells in the culture, which naturally shed proteins during growth. In another embodiment, the culture medium is polypeptide free. In another embodiment, the culture medium is serum free. In another embodiment the culture medium is animal protein free. In another embodiment the culture medium is animal component free. In another embodiment, the culture medium contains protein, e.g., animal protein from serum such as fetal calf serum. In another embodiment, the culture has recombinant proteins exogenously added. In another embodiment, the proteins are from a certified pathogen free animal. The term “chemically defined” as used herein shall mean, that the medium does not comprise any undefined supplements, such as, for example, extracts of animal components, organs, glands, plants, or yeast. Accordingly, each component of a chemically defined medium is accurately defined. In a preferred embodiment, the media are animal-component free and protein free.

In further embodiments, subsequent to purification from a mammalian cell culture, rFVIII is reconstituted prior to administration. In still further embodiments, the rVWF is treated with furin prior to or subsequent to reconstitution. In further embodiments, the furin is recombinant furin. In still further embodiments, the rVWF of the invention is not exposed to ADAMTS13, with the result that ultra large (i.e., comprising 10 or more subunits) are present in rVWF compositions of the invention.

In specific aspects, the rVWF used in methods of the present invention is contained in a formulation containing a buffer, a sugar and/or a sugar alcohol (including without limitation trehalose and mannitol), a stabilizer (such as glycine), and a surfactant (such as polysorbate 80). In further embodiments, for formulations containing rFVIII, the formulation may further include sodium, histidine, calcium, and glutathione.

In one aspect, the formulations comprising rVWF is lyophilized prior to administration. Lyophilization is carried out using techniques common in the art and should be optimized for the composition being developed [Tang et al., Pharm Res. 21:191-200. (2004) and Chang et al., Pharm Res. 13:243-9 (1996)].

Methods of preparing pharmaceutical formulations can include one or more of the following steps: adding a stabilizing agent as described herein to said mixture prior to lyophilizing, adding at least one agent selected from a bulking agent, an osmolarity regulating agent, and a surfactant, each of which as described herein, to said mixture prior to lyophilization. A lyophilized formulation is, in one aspect, at least comprised of one or more of a buffer, a bulking agent, and a stabilizer. In this aspect, the utility of a surfactant is evaluated and selected in cases where aggregation during the lyophilization step or during reconstitution becomes an issue. An appropriate buffering agent is included to maintain the formulation within stable zones of pH during lyophilization.

The standard reconstitution practice for lyophilized material is to add back a volume of pure water or sterile water for injection (WFI) (typically equivalent to the volume removed during lyophilization), although dilute solutions of antibacterial agents are sometimes used in the production of pharmaceuticals for parenteral administration [Chen, Drug Development and Industrial Pharmacy, 18:1311-1354 (1992)]. Accordingly, methods are provided for preparation of reconstituted recombinant VWF compositions comprising the step of adding a diluent to a lyophilized recombinant VWF composition of the invention.

The lyophilized material may be reconstituted as an aqueous solution. A variety of aqueous carriers, e.g., sterile water for injection, water with preservatives for multi dose use, or water with appropriate amounts of surfactants (for example, an aqueous suspension that contains the active compound in admixture with excipients suitable for the manufacture of aqueous suspensions). In various aspects, such excipients are suspending agents, for example and without limitation, sodium carboxymethylcellulose, methylcellulose, hydroxypropylmethylcellulose, sodium alginate, polyvinylpyrrolidone, gum tragacanth and gum acacia; dispersing or wetting agents are a naturally-occurring phosphatide, for example and without limitation, lecithin, or condensation products of an alkylene oxide with fatty acids, for example and without limitation, polyoxyethylene stearate, or condensation products of ethylene oxide with long chain aliphatic alcohols, for example and without limitation, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example and without limitation, polyethylene sorbitan monooleate. In various aspects, the aqueous suspensions also contain one or more preservatives, for example and without limitation, ethyl, or n-propyl, p-hydroxybenzoate.

In certain embodiments, compositions of the present invention are liquid formulations for administration with the use of a syringe or other storage vessel. In further embodiments, these liquid formulations are produced from lyophilized material described herein reconstituted as an aqueous solution.

In a further aspect, the compositions of the invention further comprise one or more pharmaceutically acceptable carriers. The phrases “pharmaceutically” or “pharmacologically” acceptable refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

II. Production of Recombinant VWF

The free mature recombinant von Willebrand Factor (rVWF) of the present invention can be produced recombinantly. One skilled in the art recognizes useful methods for expressing a recombinant protein in a host cell. In some instances, the method includes expressing a nucleic acid sequence encoding rVWF in a host cell such as a CHO cell and culturing the resulting host cell under certain conditions to produce rVWF, prepro-VWF, pro-VWF, and the like.

In certain embodiments, the nucleic acid sequence comprising a sequence coding for VWF can be an expression vector. The vector can be delivered by a virus or can be a plasmid. The nucleic acid sequence coding for the protein can be a specific gene or a biologically functional part thereof. In one embodiment, the protein is at least a biologically active part of VWF. The nucleic acid sequence can further comprise other sequences suitable for a controlled expression of a protein such as promoter sequences, enhancers, TATA boxes, transcription initiation sites, polylinkers, restriction sites, poly-A-sequences, protein processing sequences, selection markers, and the like which are generally known to a person of ordinary skill in the art.

A wide variety of vectors can be used for the expression of the VWF and can be selected from eukaryotic expression vectors. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

In some aspects, the rVWF used in the methods of the present invention is produced by expression in a mammalian cell culture using methods known in the art. In particular embodiments, the mammalian culture comprises CHO cells. In further embodiments, the rVWF is co-expressed with recombinant Factor VIII (rFVIII) in the same culture. In such embodiments, the rVWF and the rFVIII are purified together (co-purified) or separately using methods known in the art. In other embodiments, the rVWF is expressed in a culture that does not contain rFVIII.

In some embodiments, rVWF is expressed and isolated from a suitable eukaryotic host system. Examples of eukaryotic cells include, without limitation, mammalian cells, such as CHO, COS, HEK 293, BHK, SK-Hep, and HepG2; insect cells, e.g., SF9 cells, SF21 cells, S2 cells, and High Five cells; and yeast cells, e.g., Saccharomyces or Schizosaccharomyces cells. In one embodiment, the VWF can be expressed in yeast cells, insect cells, avian cells, mammalian cells, and the like. For example, in a human cell line, a hamster cell line, or a murine cell line. In one particular embodiment, the cell line is a CHO, BHK, or HEK cell line. Typically, mammalian cells, e.g., CHO cell from a continuous cell line, can be used to express the VWF of the present invention. In certain instances, VWF protein is expressed and isolated from a CHO cell expression system.

VWF can be produced in a cell culture system or according to any cell culture method recognized by those in the art. In some embodiments, the cell cultures can be performed in large bioreactors under conditions suitable for providing high volume-specific culture surface areas to achieve high cell densities and protein expression. One means for providing such growth conditions is to use microcarriers for cell-culture in stirred tank bioreactors. The concept of cell-growth on microcarriers was first described by van Wezel (van Wezel, A. L., Nature, 1967, 216:64-5) and allows for cell attachment on the surface of small solid particles suspended in the growth medium. These methods provide for high surface-to-volume ratios and thus allow for efficient nutrient utilization. Furthermore, for expression of secreted proteins in eukaryotic cell lines, the increased surface-to-volume ratio allows for higher levels of secretion and thus higher protein yields in the supernatant of the culture. Finally, these methods allow for the easy scale-up of eukaryotic expression cultures.

The cells expressing VWF can be bound to a spherical or a porous microcarrier during cell culture growth. The microcarrier can be a microcarrier selected from the group of microcarriers based on dextran, collagen, plastic, gelatine and cellulose and others as described in Butler (1988. In: Spier & Griffiths, Animal Cell Biotechnology 3:283-303). It is also possible to grow the cells to a biomass on spherical microcarriers and subculture the cells when they have reached final fermenter biomass and prior to production of the expressed protein on a porous microcarrier or vice versa. Suitable spherical microcarriers can include smooth surface microcarriers, such as Cytodex™ 1, Cytodex™ 2, and Cytodex™ 3 (GE Healthcare) and macroporous microcarriers such as Cytopore™ 1, Cytopore™ 2, Cytoline™ 1, and Cytoline™ 2 (GE Healthcare).

In a further embodiment, the VWF propeptide is cleaved from the non-mature VWF in vitro through exposure of the pro-VWF to furin. In some embodiments, the furin used for propeptide cleavage is recombinant furin.

In certain embodiments, rVWF is expressed in cells cultured in cell culture media that produces high molecular weight rVWF. The terms “cell culture solution,” “cell culture medium or media,” and “cell culture supernatant” refer to aspects of cell culture processes generally well known in the art. In the context of the present invention, a cell culture solution can include cell culture media and cell culture supernatant. The cell culture media are externally added to the cell culture solution, optionally together with supplements, to provide nutrients and other components for culturing the cells expressing VWF. The cell culture supernatant refers to a cell culture solution comprising the nutrients and other components from the cell culture medium as well as products released, metabolized, and/or excreted from the cells during culture. In further embodiments, the media can be animal protein-free and chemically defined. Methods of preparing animal protein-free and chemically defined culture media are known in the art, for example in US 2006/0094104, US 2007/0212770, and US 2008/0009040, which are both incorporated herein for all purposes and in particular for all teachings related to cell culture media. “Protein free” and related terms refers to protein that is from a source exogenous to or other than the cells in the culture, which naturally shed proteins during growth. In another embodiment, the culture medium is polypeptide free. In another embodiment, the culture medium is serum free. In another embodiment the culture medium is animal protein free. In another embodiment the culture medium is animal component free. In another embodiment, the culture medium contains protein, e.g., animal protein from serum such as fetal calf serum. In another embodiment, the culture has recombinant proteins exogenously added. In another embodiment, the proteins are from a certified pathogen free animal. The term “chemically defined” as used herein shall mean, that the medium does not comprise any undefined supplements, such as, for example, extracts of animal components, organs, glands, plants, or yeast. Accordingly, each component of a chemically defined medium is accurately defined. In a preferred embodiment, the media are animal-component free and protein free.

In certain embodiments, the culture of cells expressing VWF can be maintained for at least about 7 days, or at least about 14 days, 21 days, 28 days, or at least about 5 weeks, 6 weeks, 7 weeks, or at least about 2 months, or 3, 4, 5, 6, 7, 8, 9, 10, 11, 12, 13, 14, 15, 16, 17, 18 months or longer. The cell density at which a cell-culture is maintained at for production of a recombinant VWF protein will depend upon the culture-conditions and medium used for protein expression. One of skill in the art will readily be able to determine the optimal cell density for a cell-culture producing an VWF. In one embodiment, the culture is maintained at a cell density of between about 0.5×10⁶ and 4×10⁷ cells/ml for an extended period of time. In other embodiments, the cell density is maintained at a concentration of between about 1.0×10⁶ and about 1.0×10⁷ cells/ml for an extended period of time. In other embodiments, the cell density is maintained at a concentration of between about 1.0×10⁶ and about 4.0×10⁶ cells/ml for an extended period of time. In other embodiments, the cell density is maintained at a concentration of between about 1.0×10⁶ and about 4.0×10⁶ cells/ml for an extended period of time. In yet other embodiments, the cell density may be maintained at a concentration between about 2.0×10⁶ and about 4.0×10⁶, or between about 1.0×10⁶ and about 2.5×10⁶, or between about 1.5×10⁶ and about 3.5×10⁶, or any other similar range, for an extended period of time. After an appropriate time in cell culture, the rVWF can be isolated from the expression system using methods known in the art.

In a specific embodiment, the cell density of the continuous cell culture for production of rVWF is maintained at a concentration of no more than 2.5×10⁶ cells/mL for an extended period. In other specific embodiments, the cell density is maintained at no more than 2.0×10⁶ cells/mL, 1.5×10⁶ cells/mL, 1.0×10⁶ cells/mL, 0.5×10⁶ cells/mL, or less. In one embodiment, the cell density is maintained at between 1.5×10⁶ cells/mL and 2.5×10⁶ cells/mL.

In one embodiment of the cell cultures described above, the cell culture solution comprises a medium supplement comprising copper. Such cell culture solutions are described, for example, in U.S. Pat. Nos. 8,852,888 and 9,409,971, which is hereby incorporated by reference in its entirety for all purposes and in particular for all teachings related to cell culture methods and compositions for producing recombinant VWF.

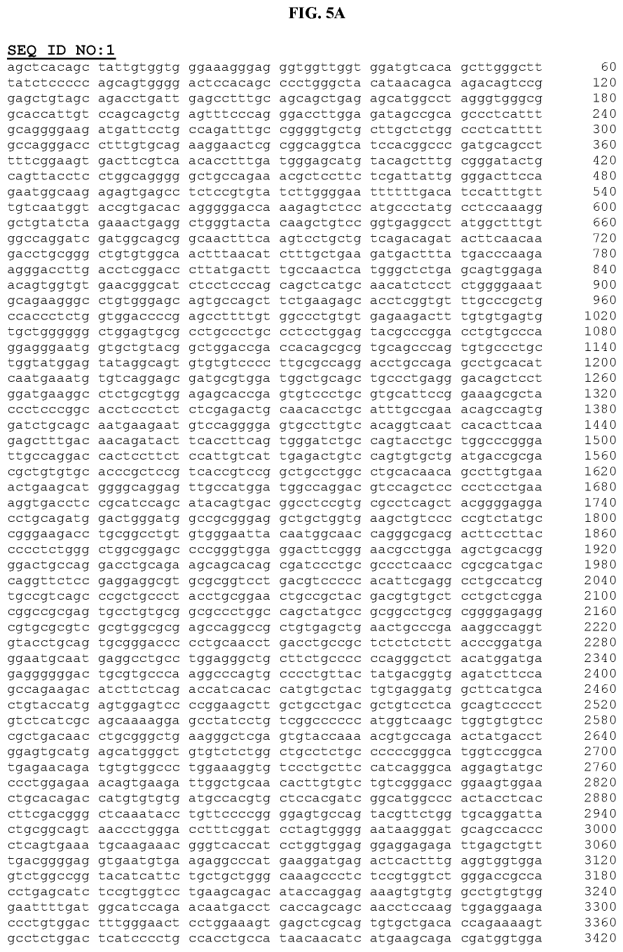

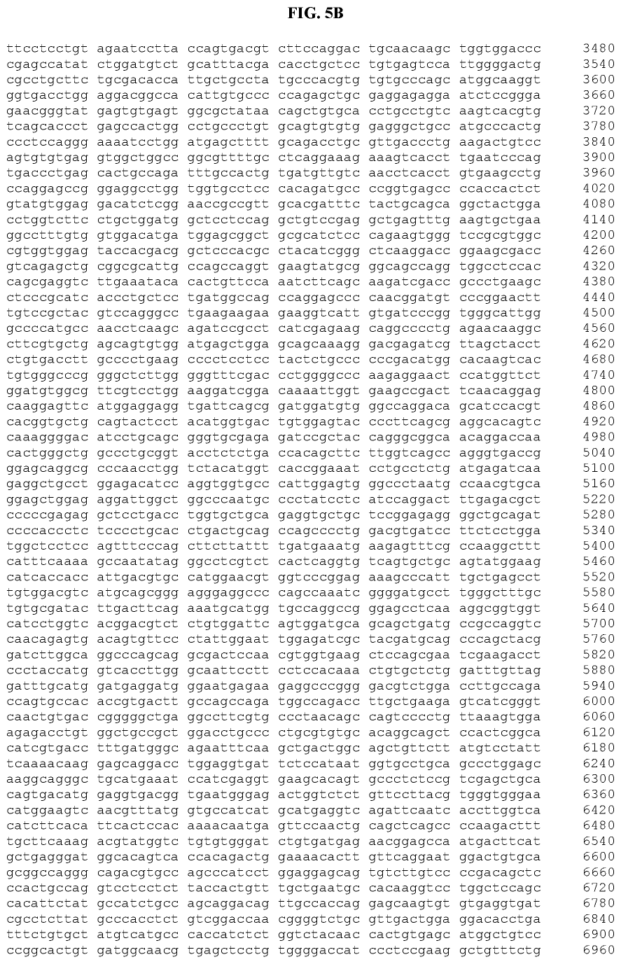

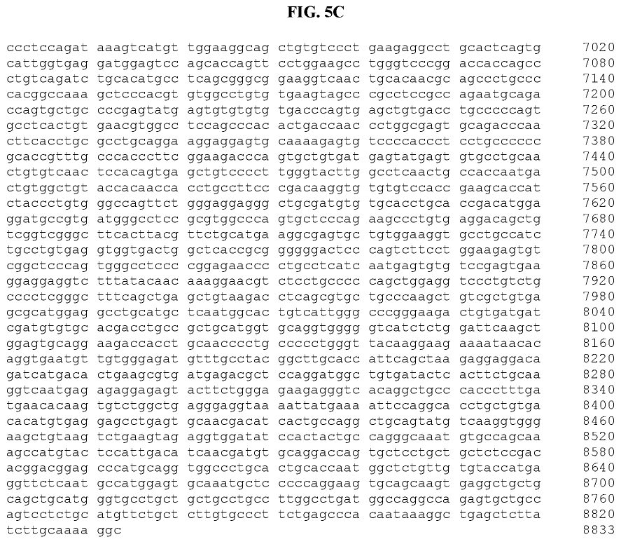

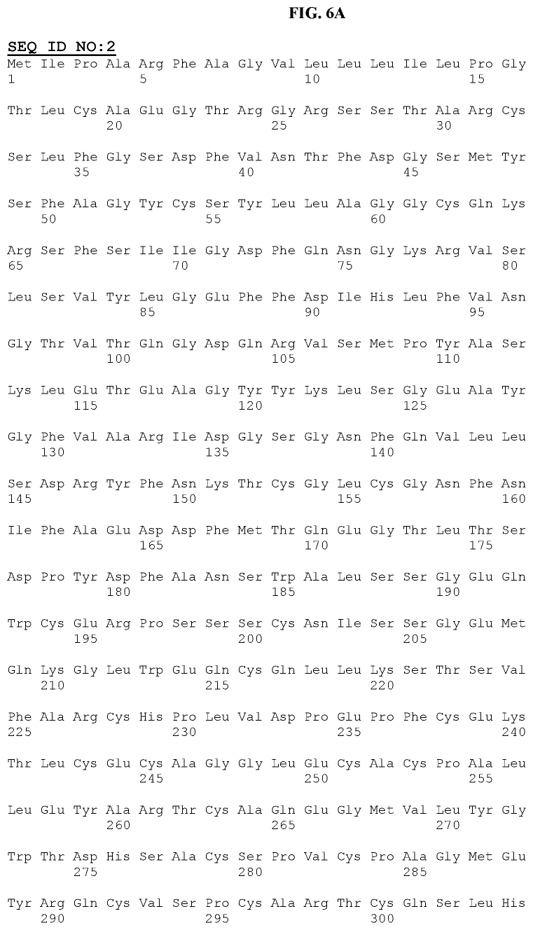

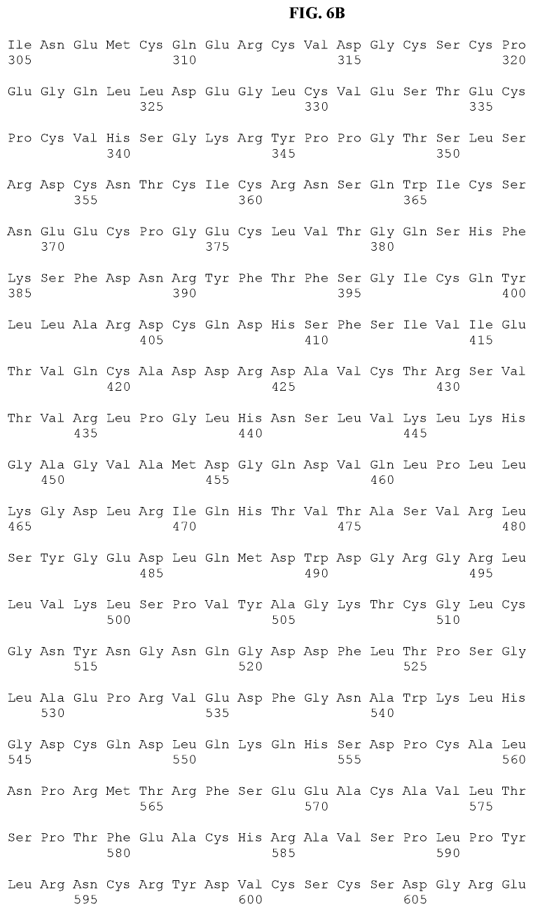

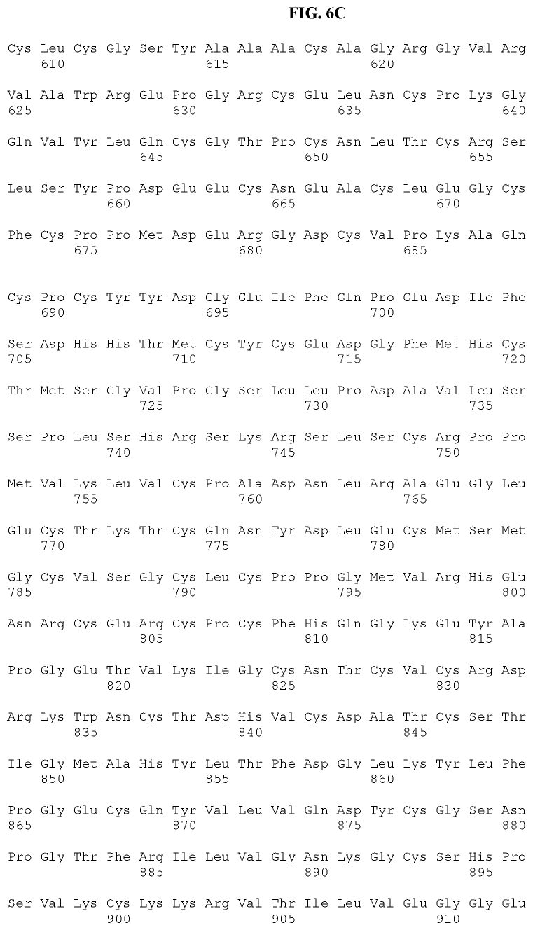

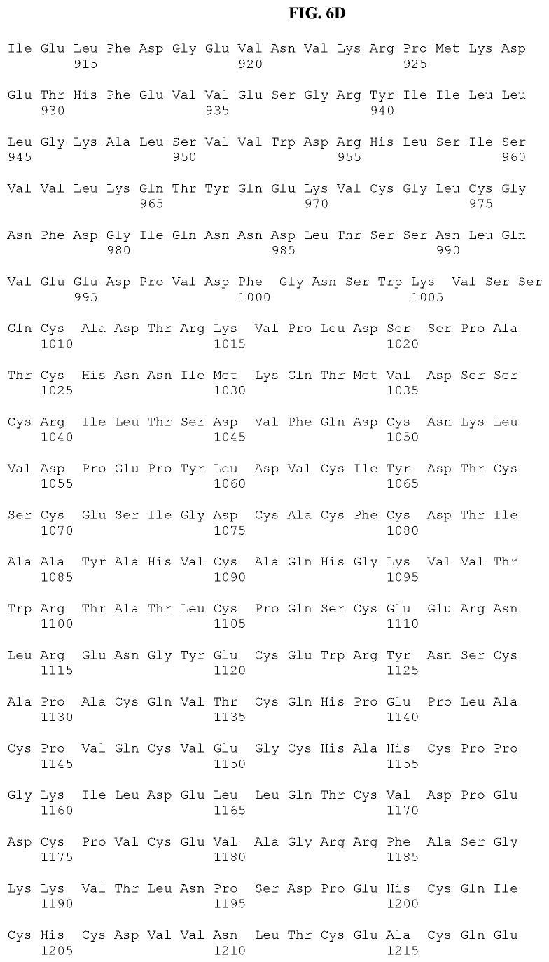

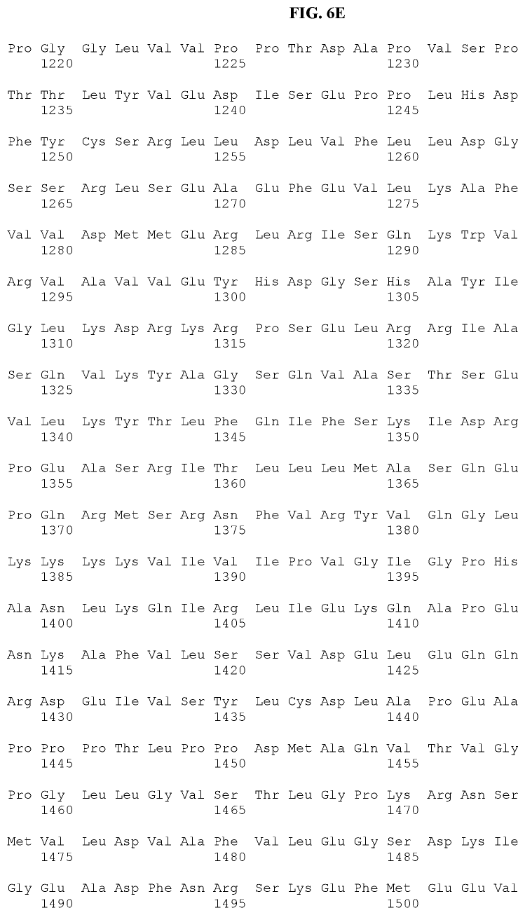

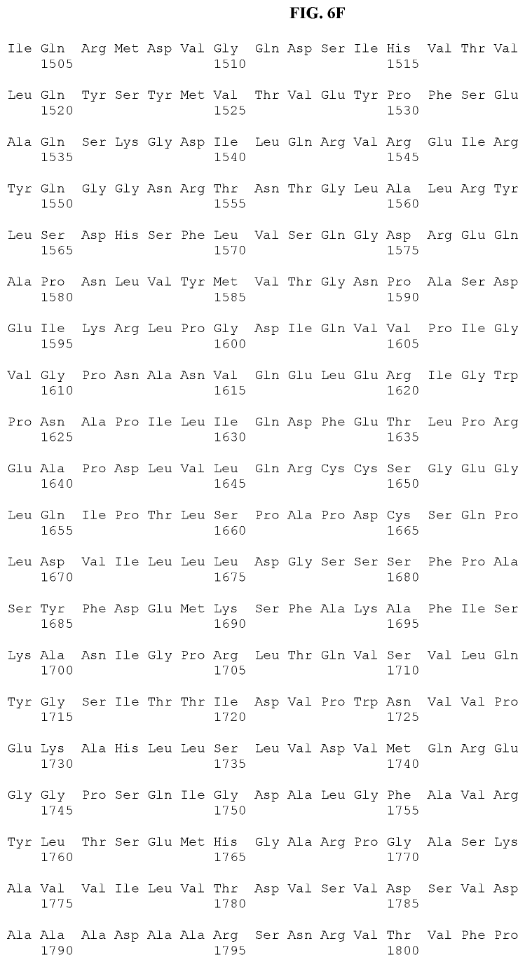

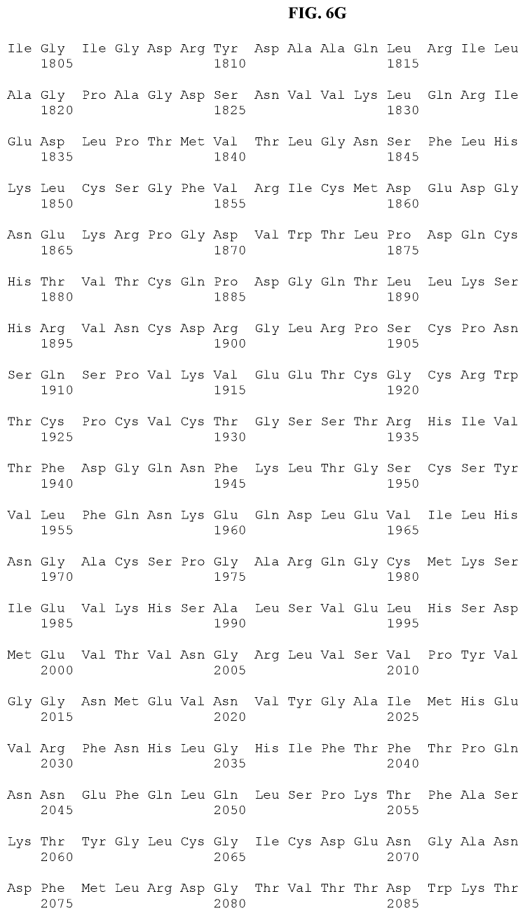

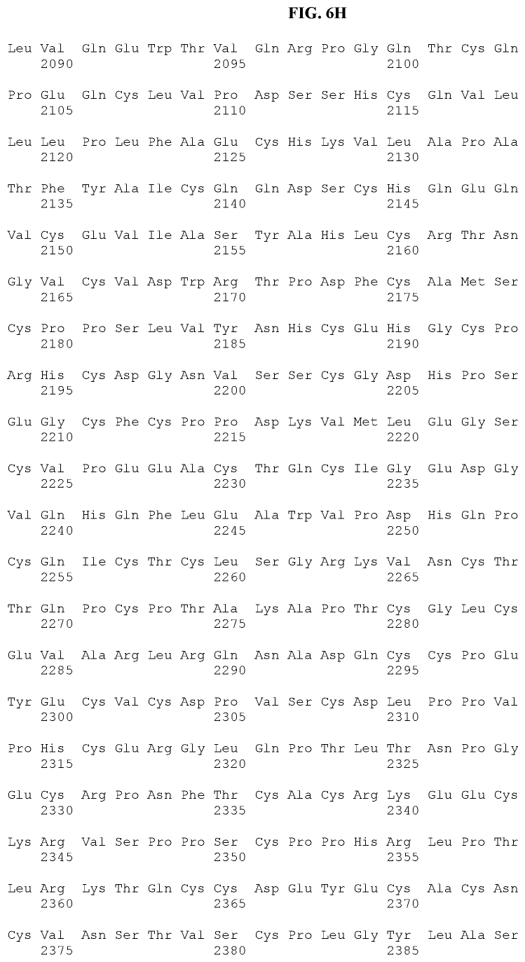

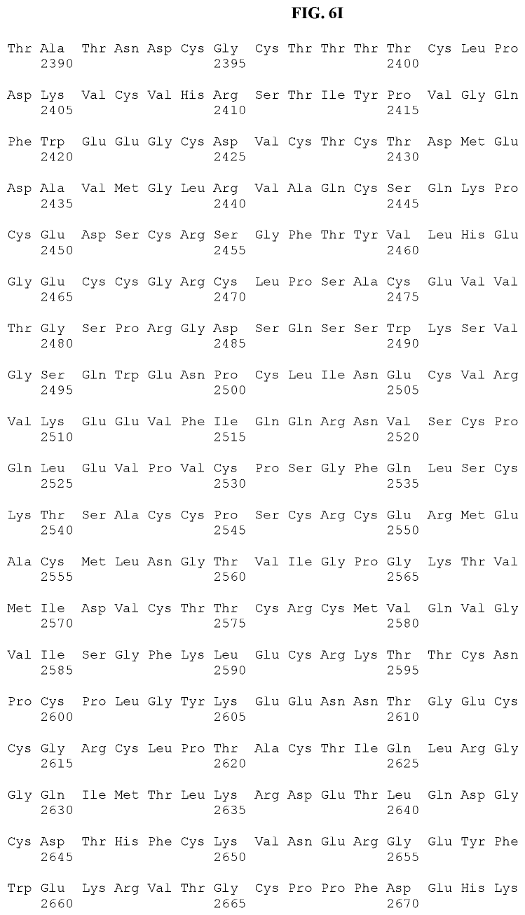

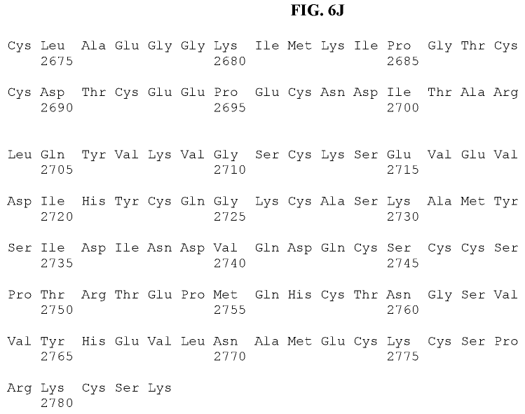

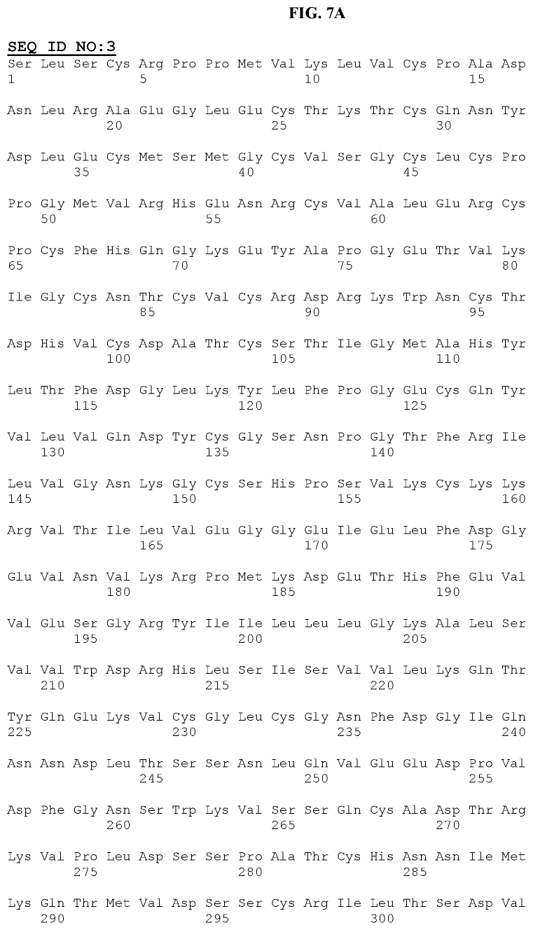

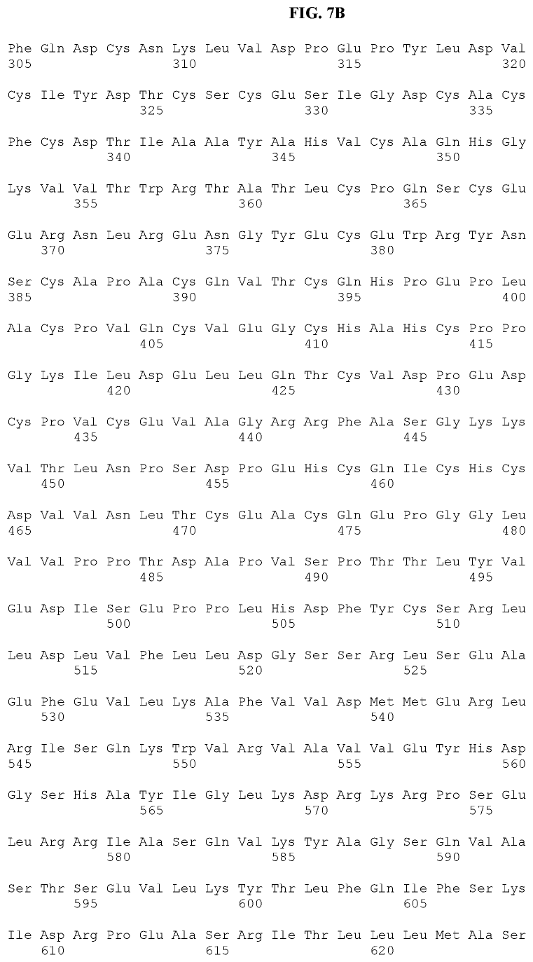

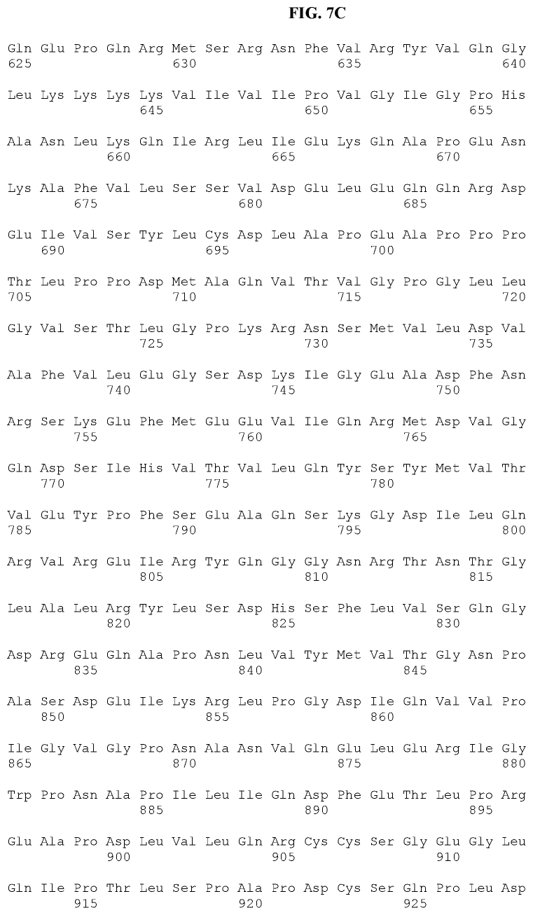

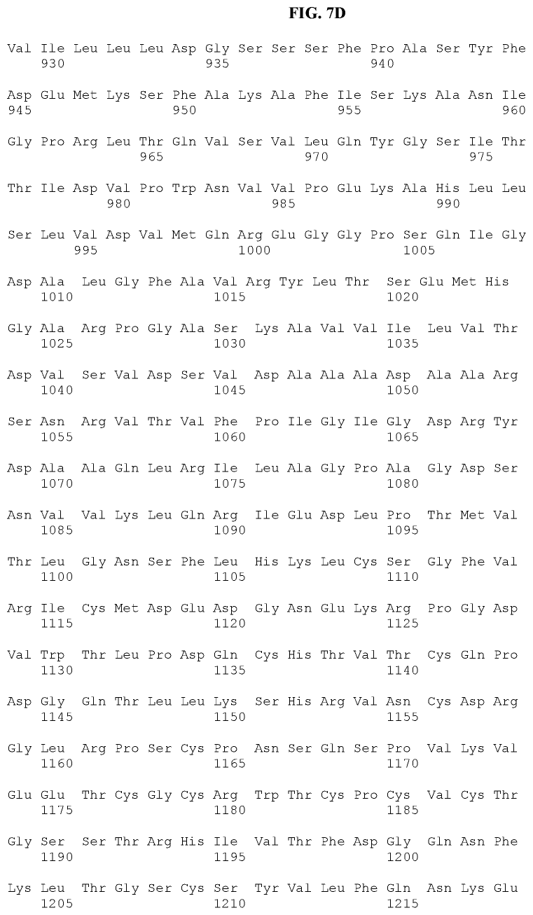

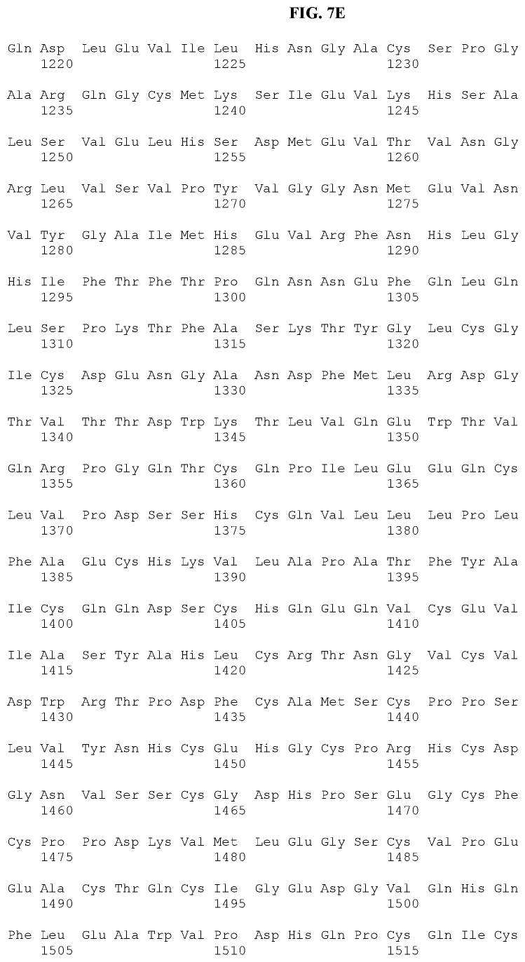

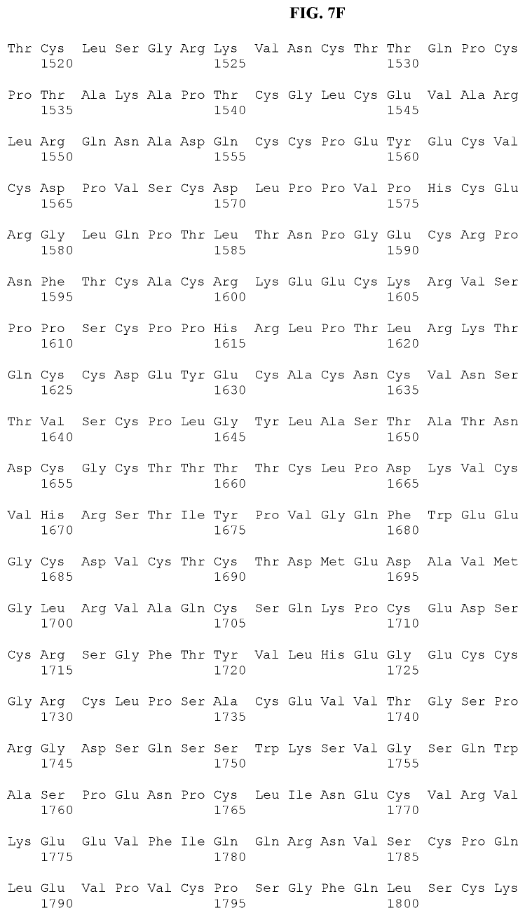

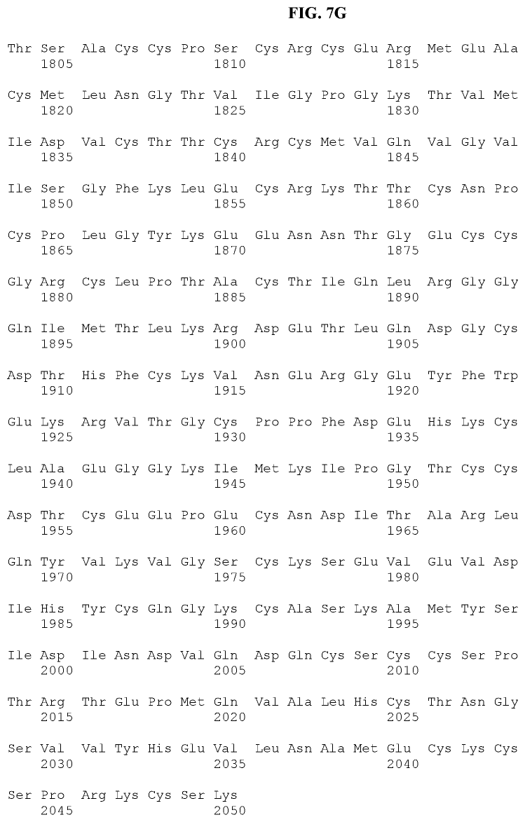

The polynucleotide and amino acid sequences of prepro-VWF are set out in SEQ ID NO:1 and SEQ ID NO:2, respectively, and are available at GenBank Accession Nos. NM_000552 (Homo sapiens von Willebrand factor (VWF) mRNA) and NP 000543, respectively. The amino acid sequence corresponding to the mature VWF protein is set out in SEQ ID NO: 3 (corresponding to amino acids 764-2813 of the full length prepro-VWF amino acid sequence). In some embodiments, the VWF exhibits at least 80%, at least 85%, at least 90%, at least 95%, at least 96%, at least 97%, at least 98%, at least 99%, or at least 100% identity to the sequence of SEQ ID NO:3. In some embodiments, the rVWF of the invention exhibits at least 80%, at least 85%, at least 90%, at least 95%, at least 96%, at least 97%, at least 98%, at least 99%, or at least 100% identity to the sequence of SEQ ID NO:3. See, for example, U.S. Pat. No. 8,597,910, U.S. Patent Publication No. 2016/0129090, as well as FIGS. 5A-5C, 6A-6J, and 7A-7G.

One form of useful rVWF has at least the property of in vivo-stabilizing, e.g. binding, of at least one Factor VIII (FVIII) molecule and having optionally a glycosylation pattern which is pharmacologically acceptable. Specific examples thereof include VWF without the A2 domain thus resistant to proteolysis (Lankhof et al., Thromb. Haemost. 77: 1008-1013, 1997), and a VWF fragment from Val 449 to Asn 730 including the glycoprotein 1b-binding domain and binding sites for collagen and heparin (Pietu et al., Biochem. Biophys. Res. Commun. 164: 1339-1347, 1989). The determination of the ability of a VWF to stabilize at least one FVIII molecule is, in one aspect, carried out in VWF-deficient mammals according to methods known in the state in the art.

The rVWF of the present invention can be produced by any method known in the art. One specific example is disclosed in WO86/06096 published on Oct. 23, 1986 and U.S. patent application Ser. No. 07/559,509, filed on Jul. 23, 1990, which is incorporated herein by reference with respect to the methods of producing recombinant VWF. Thus, methods are known in the art for (i) the production of recombinant DNA by genetic engineering, e.g. via reverse transcription of RNA and/or amplification of DNA, (ii) introducing recombinant DNA into prokaryotic or eukaryotic cells by transfection, e.g. via electroporation or microinjection, (iii) cultivating the transformed cells, e.g. in a continuous or batchwise manner, (iv) expressing VWF, e.g. constitutively or upon induction, and (v) isolating the VWF, e.g. from the culture medium or by harvesting the transformed cells, in order to (vi) obtain purified rVWF, e.g. via anion exchange chromatography or affinity chromatography. A recombinant VWF is, in one aspect, made in transformed host cells using recombinant DNA techniques well known in the art. For instance, sequences coding for the polypeptide could be excised from DNA using suitable restriction enzymes. Alternatively, the DNA molecule is, in another aspect, synthesized using chemical synthesis techniques, such as the phosphoramidate method. Also, in still another aspect, a combination of these techniques is used.

The invention also provides vectors encoding polypeptides of the invention in an appropriate host. The vector comprises the polynucleotide that encodes the polypeptide operatively linked to appropriate expression control sequences. Methods of effecting this operative linking, either before or after the polynucleotide is inserted into the vector, are well known. Expression control sequences include promoters, activators, enhancers, operators, ribosomal binding sites, start signals, stop signals, cap signals, polyadenylation signals, and other signals involved with the control of transcription or translation. The resulting vector having the polynucleotide therein is used to transform an appropriate host. This transformation may be performed using methods well known in the art.

Any of a large number of available and well-known host cells are used in the practice of this invention. The selection of a particular host is dependent upon a number of factors recognized by the art, including, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all host cells are equally effective for the expression of a particular DNA sequence. Within these general guidelines, useful microbial host cells include, without limitation, bacteria, yeast and other fungi, insects, plants, mammalian (including human) cells in culture, or other hosts known in the art.

Transformed host cells are cultured under conventional fermentation conditions so that the desired compounds are expressed. Such fermentation conditions are well known in the art. Finally, the polypeptides are purified from culture media or the host cells themselves by methods well known in the art.

Depending on the host cell utilized to express a compound of the invention, carbohydrate (oligosaccharide) groups are optionally attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids not counting proline. The structures of N-linked and O-linked oligosaccharides and the sugar residues found in each type are different. One type of sugar that is commonly found on both N-linked and O-linked oligosaccharides is N-acetylneuraminic acid (referred to as sialic acid). Sialic acid is usually the terminal residue of both N-linked and O-linked oligosaccharides and, by virtue of its negative charge, in one aspect, confers acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). In other aspects, such sites are glycosylated by synthetic or semi-synthetic procedures known in the art.

In some embodiments, sialysation (also referred to as sialylation), can be performed on the column as part of the purification procedures described herein (including the anion exchange, cation exchange, size exclusion, and/or immunoaffinity methods). In some embodiments, the sialylation results in increased stability of the rVWF as compared to rVWF that has not undergone sialylation. In some embodiments, the sialylation results in increased stability of the rVWF in blood circulation (for example, after administration to a subject) as compared to rVWF that has not undergone sialylation. In some embodiments, the increased stability of salivated rVWF results in an increase of 10%, 20%, 30%, 40%, 50%, 60%, 70%, 80%, 90%, or more as compared rVWF that has not undergone sialylation. In some embodiments, the sialylation results in increased half-life for the rVWF as compared to rVWF that has not undergone sialylation. In some embodiments, the sialylation results in increased half-life for the rVWF in blood circulation (for example, after administration to a subject) as compared to rVWF that has not undergone sialylation. In some embodiments, the increased half-life of sialylated rVWF results in an increase of 10%, 20%, 30%, 40%, 50%, 60%, 70%, 80%, 90%, or more as compared rVWF that has not undergone sialylation. In some embodiments, the increased half-life of sialylated rVWF results in rVWF that is stable for 1 hour, 2 hours, 3 hours, 4 hours, 6 hours, 12 hours, 24 hours or more in blood circulation (for example, after administration to a subject) as compared to rVWF that has not undergone sialylation. In some embodiments, sialylation increases the number of 2,3 sialylation and/or 2,6 sialylation. In some embodiments, sialylation is increased by the addition of 2,3 sialyltransferase and/or 2,6 sialyltransferase and CMP-NANA (Cytidine-5′-monophospho-N-acetylneuraminic acid sodium salt) as an additional buffer step. In some embodiments, sialylation is increased by the addition of 2,3 sialyltransferase and CMP-NANA (Cytidine-5′-monophospho-N-acetylneuraminic acid sodium salt) as an additional buffer step. In some embodiments, 2,3 sialylation is increased by the addition of 2,3 sialyltransferase and CMP-NANA (Cytidine-5′-monophospho-N-acetylneuraminic acid sodium salt) as an additional buffer step.

In some embodiments, 2,6 sialylation is increased by the addition of 2,6 sialyltransferase and CMP-NANA (Cytidine-5′-monophospho-N-acetylneuraminic acid sodium salt) as an additional buffer step. In some embodiments, 2,3 sialylation and/or 2,6 sialylation are increased by the addition of 2,3 sialyltransferase and/or 2,6 sialyltransferase and CMP-NANA (Cytidine-5′-monophospho-N-acetylneuraminic acid sodium salt) as an additional buffer step. In some embodiments, CMP-NANA is chemically or enzymatic modified to transfer modified sialic acid to potential free position. In some embodiments, sialylation is performed by loading rVWF onto the resin, washing with one or more buffers as described herein to deplete unwanted impurities, apply one or more buffers containing sialyltransferase and CMP-NANA at conditions that allow additional sialylation, and washing with one or more buffers to deplete excess of the sialylation reagents, and eluting with one or more buffers the enhanced rVWF (e.g., the rVWF with increased sialylation). In some embodiments, the sialylation process is performed as part of a cation exchange method, an anion exchange method, a size exclusion method, or an immunoaffinity purification method, as described herein.

Alternatively, the compounds are made by synthetic methods using, for example, solid phase synthesis techniques. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides

Fragments, variants and analogs of VWF can be produced according to methods that are well-known in the art. Fragments of a polypeptide can be prepared using, without limitation, enzymatic cleavage (e.g., trypsin, chymotrypsin) and also using recombinant means to generate a polypeptide fragments having a specific amino acid sequence. Polypeptide fragments may be generated comprising a region of the protein having a particular activity, such as a multimerization domain or any other identifiable VWF domain known in the art.

Methods of making polypeptide analogs are also well-known. Amino acid sequence analogs of a polypeptide can be substitutional, insertional, addition or deletion analogs. Deletion analogs, including fragments of a polypeptide, lack one or more residues of the native protein which are not essential for function or immunogenic activity. Insertional analogs involve the addition of, e.g., amino acid(s) at a non-terminal point in the polypeptide. This analog may include, for example and without limitation, insertion of an immunoreactive epitope or simply a single residue. Addition analogs, including fragments of a polypeptide, include the addition of one or more amino acids at either or both termini of a protein and include, for example, fusion proteins. Combinations of the aforementioned analogs are also contemplated.

Substitutional analogs typically exchange one amino acid of the wild-type for another at one or more sites within the protein, and may be designed to modulate one or more properties of the polypeptide without the complete loss of other functions or properties. In one aspect, substitutions are conservative substitutions. “Conservative amino acid substitution” is substitution of an amino acid with an amino acid having a side chain or a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

In one aspect, analogs are substantially homologous or substantially identical to the recombinant VWF from which they are derived. Analogs include those which retain at least some of the biological activity of the wild-type polypeptide, e.g. blood clotting activity.

Polypeptide variants contemplated include, without limitation, polypeptides chemically modified by such techniques as ubiquitination, glycosylation, including polysialation (or polysialylation), conjugation to therapeutic or diagnostic agents, labeling, covalent polymer attachment such as pegylation (derivatization with polyethylene glycol), introduction of non-hydrolyzable bonds, and insertion or substitution by chemical synthesis of amino acids such as ornithine, which do not normally occur in human proteins. Variants retain the same or essentially the same binding properties of non-modified molecules of the invention. Such chemical modification may include direct or indirect (e.g., via a linker) attachment of an agent to the VWF polypeptide. In the case of indirect attachment, it is contemplated that the linker may be hydrolyzable or non-hydrolyzable.

Preparing pegylated polypeptide analogs will in one aspect comprise the steps of (a) reacting the polypeptide with polyethylene glycol (such as a reactive ester or aldehyde derivative of PEG) under conditions whereby the binding construct polypeptide becomes attached to one or more PEG groups, and (b) obtaining the reaction product(s). In general, the optimal reaction conditions for the acylation reactions are determined based on known parameters and the desired result. For example, the larger the ratio of PEG: protein, the greater the percentage of poly-pegylated product. In some embodiments, the binding construct has a single PEG moiety at the N-terminus. Polyethylene glycol (PEG) may be attached to the blood clotting factor to, for example, provide a longer half-life in vivo. The PEG group may be of any convenient molecular weight and is linear or branched. The average molecular weight of the PEG ranges from about 2 kiloDalton (“kD”) to about 100 kDa, from about 5 kDa to about 50 kDa, or from about 5 kDa to about 10 kDa. In certain aspects, the PEG groups are attached to the blood clotting factor via acylation or reductive alkylation through a natural or engineered reactive group on the PEG moiety (e.g., an aldehyde, amino, thiol, or ester group) to a reactive group on the blood clotting factor (e.g., an aldehyde, amino, or ester group) or by any other technique known in the art.

Methods for preparing polysialylated polypeptide are described in United States Patent Publication 20060160948, Fernandes et Gregoriadis; Biochim. Biophys. Acta 1341: 26-34, 1997, and Saenko et al., Haemophilia 12:42-51, 2006. Briefly, a solution of colominic acid (CA) containing 0.1 M NaIO₄ is stirred in the dark at room temperature to oxidize the CA. The activated CA solution is dialyzed against, e.g., 0.05 M sodium phosphate buffer, pH 7.2 in the dark and this solution was added to a rVWF solution and incubated for 18 h at room temperature in the dark under gentle shaking. Free reagents are optionally separated from the rVWF-polysialic acid conjugate by, for example, ultrafiltration/diafiltration. Conjugation of rVWF with polysialic acid is achieved using glutaraldehyde as cross-linking reagent (Migneault et al., Biotechniques 37: 790-796, 2004).

It is also contemplated in another aspect that prepro-VWF and pro-VWF polypeptides will provide a therapeutic benefit in the formulations of the present invention. For example, U.S. Pat. No. 7,005,502 describes a pharmaceutical preparation comprising substantial amounts of pro-VWF that induces thrombin generation in vitro. In addition to recombinant, biologically active fragments, variants, or other analogs of the naturally-occurring mature VWF, the present invention contemplates the use of recombinant biologically active fragments, variants, or analogs of the prepro-VWF (set out in SEQ ID NO:2) or pro-VWF polypeptides (amino acid residues 23 to 764 of SEQ ID NO: 2), or rVWF (set out in SEQ ID NO:3) in the formulations described herein.

Polynucleotides encoding fragments, variants and analogs may be readily generated by a worker of skill to encode biologically active fragments, variants, or analogs of the naturally-occurring molecule that possess the same or similar biological activity to the naturally-occurring molecule. In various aspects, these polynucleotides are prepared using PCR techniques, digestion/ligation of DNA encoding molecule, and the like. Thus, one of skill in the art will be able to generate single base changes in the DNA strand to result in an altered codon and a missense mutation, using any method known in the art, including, but not limited to site-specific mutagenesis. As used herein, the phrase “moderately stringent hybridization conditions” means, for example, hybridization at 42° C. in 50% formamide and washing at 60° C. in 0.1×SSC, 0.1% SDS. It is understood by those of skill in the art that variation in these conditions occurs based on the length and GC nucleotide base content of the sequences to be hybridized. Formulas standard in the art are appropriate for determining exact hybridization conditions. See Sambrook et al., 9.47-9.51 in Molecular Cloning, Cold Spring Harbor Laboratory Press, Cold Spring Harbor, N.Y. (1989).

A. VWF Multimers

Assessment of the number and percentage of rVWF multimers can be conducted using methods known in the art, including without limitation methods using electrophoresis and size exclusion chromatography methods to separate VWF multimers by size, for example as discussed by Cumming et al., (J Clin Pathol., 1993 May; 46(5): 470-473, which is hereby incorporated by reference in its entirety for all purposes and in particular for all teachings related to assessment of VWF multimers). Such techniques may further include immunoblotting techniques (such as Western Blot), in which the gel is immunoblotted with a radiolabelled antibody against VWF followed by chemiluminescent detection (see, for example, Wen et al., J. Clin. Lab. Anal., 1993, 7: 317-323, which is hereby incorporated by reference in its entirety for all purposes and in particular for all teachings related to assessment of VWF multimers). Further assays for VWF include VWF:Antigen (VWF:Ag), VWF:Ristocetin Cofactor (VWF:RCof), and VWF:Collagen Binding Activity assay (VWF:CBA), which are often used for diagnosis and classification of Von Willebrand Disease (see, for example, Favaloro et al., Pathology, 1997, 29(4): 341-456; Sadler, J E, Annu Rev Biochem, 1998, 67:395-424; and Turecek et al., Semin Thromb Hemost, 2010, 36:510-521, which are hereby incorporated by reference in their entirety for all purposes and in particular for all teachings related to assays for VWF). In some embodiments, the rVWF obtained using the present methods includes any multimer pattern present in the loading sample of the rVWF. In some embodiments, the rVWF obtained using the present methods includes physiological occurring multimer patters as well as ultralarge VWF-multimer patterns.

b. VWF Assays

In primary hemostasis VWF serves as a bridge between platelets and specific components of the extracellular matrix, such as collagen. The biological activity of VWF in this process can be measured by different in vitro assays (Turecek et al., Semin Thromb Hemost, 2010, 36: 510-521).

The VWF:Ristocetin Cofactor (VWF:RCo) assay is based on the agglutination of fresh or formalin-fixed platelets induced by the antibiotic ristocetin in the presence of VWF. The degree of platelet agglutination depends on the VWF concentration and can be measured by the turbidimetric method, e.g., by use of an aggregometer (Weiss et al., J. Clin. Invest., 1973, 52: 2708-2716; Macfarlane et al., Thromb. Diath. Haemorrh., 1975, 34: 306-308). As provided herein, the specific ristocetin cofactor activity of the VWF (VWF:RCo) of the present invention is generally described in terms of mU/μg of VWF, as measured using in vitro assays.

In some embodiments, the rVWF purified according to the methods of the present invention has a specific activity of at least about 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 105, 110, 115, 120, 125, 130, 135, 140, 145, 150 or more mU/μg. In some embodiments, rVWF used in the methods described herein has a specific activity of from 20 mU/μg to 150 mU/μg. In some embodiments, the rVWF has a specific activity of from 30 mU/μg to 120 mU/μg. In some embodiments, the rVWF has a specific activity from 40 mU/μg to 90 mU/μg. In some embodiments, the rVWF has a specific activity selected from variations 1 to 133 found in Table 3, below.

TABLE 3
Exemplary embodiments for the specific activity of rVWF found
in the compositions and used in the methods provided herein.

| (mU/μg) | |
|---|---|
| 20 | Var. 1 |
| 22.5 | Var. 2 |
| 25 | Var. 3 |
| 27.5 | Var. 4 |
| 30 | Var. 5 |
| 32.5 | Var. 6 |
| 35 | Var. 7 |
| 37.5 | Var. 8 |
| 40 | Var. 9 |
| 42.5 | Var. 10 |
| 45 | Var. 11 |
| 47.5 | Var. 12 |
| 50 | Var. 13 |
| 52.5 | Var. 14 |
| 55 | Var. 15 |
| 57.5 | Var. 16 |
| 60 | Var. 17 |
| 62.5 | Var. 18 |
| 65 | Var. 19 |
| 67.5 | Var. 20 |
| 70 | Var. 21 |
| 72.5 | Var. 22 |
| 75 | Var. 23 |
| 77.5 | Var. 24 |
| 80 | Var. 25 |
| 82.5 | Var. 26 |
| 85 | Var. 27 |
| 87.5 | Var. 28 |
| 90 | Var. 29 |
| 92.5 | Var. 30 |
| 95 | Var. 31 |
| 97.5 | Var. 32 |
| 100 | Var. 33 |
| 105 | Var. 34 |
| 110 | Var. 35 |
| 115 | Var. 36 |
| 120 | Var. 37 |
| 125 | Var. 38 |
| 130 | Var. 39 |
| 135 | Var. 40 |
| 140 | Var. 41 |
| 145 | Var. 42 |
| 150 | Var. 43 |
| 20-150 | Var. 44 |
| 20-140 | Var. 45 |
| 20-130 | Var. 46 |
| 20-120 | Var. 47 |
| 20-110 | Var. 48 |
| 20-100 | Var. 49 |
| 20-90 | Var. 50 |
| 20-80 | Var. 51 |
| 20-70 | Var. 52 |
| 20-60 | Var. 53 |
| 20-50 | Var. 54 |
| 20-40 | Var. 55 |
| 30-150 | Var. 56 |
| 30-140 | Var. 57 |
| 30-130 | Var. 58 |
| 30-120 | Var. 59 |
| 30-110 | Var. 60 |
| 30-100 | Var. 61 |
| 30-90 | Var. 62 |
| 30-80 | Var. 63 |
| 30-70 | Var. 64 |
| 30-60 | Var. 65 |
| 30-50 | Var. 66 |
| 30-40 | Var. 67 |
| 40-150 | Var. 68 |
| 40-140 | Var. 69 |
| 40-130 | Var. 70 |
| 40-120 | Var. 71 |
| 40-110 | Var. 72 |
| 40-100 | Var. 73 |
| 40-90 | Var. 74 |
| 40-80 | Var. 75 |
| 40-70 | Var. 76 |
| 40-60 | Var. 77 |
| 40-50 | Var. 78 |
| 50-150 | Var. 79 |
| 50-140 | Var. 80 |
| 50-130 | Var. 81 |
| 50-120 | Var. 82 |
| 50-110 | Var. 83 |
| 50-100 | Var. 84 |
| 50-90 | Var. 85 |
| 50-80 | Var. 86 |
| 50-70 | Var. 87 |
| 50-60 | Var. 88 |
| 60-150 | Var. 89 |
| 60-140 | Var. 90 |
| 60-130 | Var. 91 |
| 60-120 | Var. 92 |
| 60-110 | Var. 93 |
| 60-100 | Var. 94 |
| 60-90 | Var. 95 |
| 60-80 | Var. 96 |
| 60-70 | Var. 97 |
| 70-150 | Var. 98 |
| 70-140 | Var. 99 |
| 70-130 | Var. 100 |
| 70-120 | Var. 101 |
| 70-110 | Var. 102 |
| 70-100 | Var. 103 |
| 70-90 | Var. 104 |
| 70-80 | Var. 105 |
| 80-150 | Var. 106 |
| 80-140 | Var. 107 |
| 80-130 | Var. 108 |
| 80-120 | Var. 109 |
| 80-110 | Var. 110 |
| 80-100 | Var. 111 |
| 80-90 | Var. 112 |
| 90-150 | Var. 113 |
| 90-140 | Var. 114 |
| 90-130 | Var. 115 |
| 90-120 | Var. 116 |
| 90-110 | Var. 117 |
| 90-100 | Var. 118 |
| 100-150 | Var. 119 |
| 100-140 | Var. 120 |
| 100-130 | Var. 121 |
| 100-120 | Var. 122 |
| 100-110 | Var. 123 |
| 110-150 | Var. 124 |
| 110-140 | Var. 125 |
| 110-130 | Var. 126 |
| 110-120 | Var. 127 |
| 120-150 | Var. 128 |
| 120-140 | Var. 129 |
| 120-130 | Var. 130 |
| 130-150 | Var. 131 |
| 130-140 | Var. 132 |
| 140-150 | Var. 133 |

Var. = Variation

The rVWF of the present invention is highly multimeric comprising about 10 to about 40 subunits. In further embodiments, the multimeric rVWF produced using methods of the present invention comprise about 10-30, 12-28, 14-26, 16-24, 18-22, 20-21 subunits. In some embodiments, the rVWF is present in multimers varying in size from dimers to multimers of over 40 subunits (>10 million Daltons). The largest multimers provide multiple binding sites that can interact with both platelet receptors and subendothelial matrix sites of injury, and are the most hemostatically active form of VWF. In some embodiments, the rVWF of the present invention comprises ultralarge multimers (ULMs). Generally, high and ultralarge multimers are considered to be hemostatically most effective (see, for example, Turecek, P., Hämostaseologie, (Vol. 37): Supplement 1, pages S15-S25 (2017)). In some embodiments, the rVWF is between 500 kDa and 20,000 kDa. In some embodiments, any desired multimer pattern can be obtained using the methods described. In some embodiments, when anion exchange and/or cation exchanger methods are employed, the pH, conductivity, and/or counterion concentration of the buffers in the one or more wash step(s) or the gradient buffers can be manipulated to obtain the desired multimer pattern. In some embodiments, then size exclusion chromatography methods are employed, the collection criteria can be employed to obtain the desired multimer pattern. In some embodiments, the described multimer pattern comprises ultralarge multimers. In some embodiments, the ultralarge multimers are at least 10,000 kDa, at least 11,000 kDa, at least 12,000 kDa, at least 13,000 kDa, at least 14,000 kDa, at least 15,000 kDa, at least 16,000 kDa, at least 17,000 kDa, at least 18,000 kDa, at least 19,000 kDa, at least 20,000 kDa. In some embodiments, the ultralarge multimers are between about 10,000 kDa and 20,000 kDa. In some embodiments, the ultralarge multimers are between about 11,000 kDa and 20,000 kDa. In some embodiments, the ultralarge multimers are between about 12,000 kDa and 20,000 kDa. In some embodiments, the ultralarge multimers are between about 13,000 kDa and 20,000 kDa. In some embodiments, the ultralarge multimers are between about 14,000 kDa and 20,000 kDa. In some embodiments, the ultralarge multimers are between about 15,000 kDa and 20,000 kDa. In some embodiments, the ultralarge multimers are between about 16,000 kDa and 20,000 kDa. In some embodiments, the ultralarge multimers are between about 17,000 kDa and 20,000 kDa. In some embodiments, the ultralarge multimers are between about 18,000 kDa and 20,000 kDa. In some embodiments, the ultralarge multimers are between about 19,000 kDa and 20,000 kDa. In some embodiments, the rVWF obtained using the present methods includes any multimer pattern present in the loading sample of the rVWF. In some embodiments, the rVWF obtained using the present methods includes physiolocical occurring multimer patters as well as ultra large VWF-multimer patterns.

In some embodiments, the rVWF composition prepared by the purification method described herein has a distribution of rVWF oligomers characterized in that 95% of the oligomers have between 6 subunits and 20 subunits. In some embodiments, the rVWF composition has a distribution of rVWF oligomers characterized in that 95% of the oligomers have a range of subunits selected from variations 458 to 641 found in 4.

TABLE 4
Exemplary embodiments for the distribution of rVWF oligomers found
in the compositions and used in the methods provided herein.

| Subunits | |
|---|---|
| 2-40 | Var. 458 |
| 2-38 | Var. 459 |
| 2-36 | Var. 460 |
| 2-34 | Var. 461 |
| 2-32 | Var. 462 |
| 2-30 | Var. 463 |
| 2-28 | Var. 464 |
| 2-26 | Var. 465 |
| 2-24 | Var. 466 |
| 2-22 | Var. 467 |
| 2-20 | Var. 468 |
| 2-18 | Var. 469 |
| 2-16 | Var. 470 |
| 2-14 | Var. 471 |
| 2-12 | Var. 472 |
| 2-10 | Var. 473 |
| 2-8 | Var. 474 |
| 4-40 | Var. 475 |
| 4-38 | Var. 476 |
| 4-36 | Var. 477 |
| 4-34 | Var. 478 |
| 4-32 | Var. 479 |
| 4-30 | Var. 480 |
| 4-28 | Var. 481 |
| 4-26 | Var. 482 |
| 4-24 | Var. 483 |
| 4-22 | Var. 484 |
| 4-20 | Var. 485 |
| 4-18 | Var. 486 |
| 4-16 | Var. 487 |
| 4-14 | Var. 488 |
| 4-12 | Var. 489 |
| 4-10 | Var. 490 |
| 4-8 | Var. 491 |
| 6-40 | Var. 492 |
| 6-38 | Var. 493 |
| 6-36 | Var. 494 |
| 6-34 | Var. 495 |
| 6-32 | Var. 496 |
| 6-30 | Var. 497 |
| 6-28 | Var. 498 |
| 6-26 | Var. 499 |
| 6-24 | Var. 500 |
| 6-22 | Var. 501 |
| 6-20 | Var. 502 |
| 6-18 | Var. 503 |
| 6-16 | Var. 504 |
| 6-14 | Var. 505 |
| 6-12 | Var. 506 |
| 6-10 | Var. 507 |
| 6-8 | Var. 508 |
| 8-40 | Var. 509 |
| 8-38 | Var. 510 |
| 8-36 | Var. 511 |
| 8-34 | Var. 512 |
| 8-32 | Var. 513 |
| 8-30 | Var. 514 |
| 8-28 | Var. 515 |
| 8-26 | Var. 516 |
| 8-24 | Var. 517 |
| 8-22 | Var. 518 |
| 8-20 | Var. 519 |
| 8-18 | Var. 520 |
| 8-16 | Var. 521 |
| 8-14 | Var. 522 |
| 8-12 | Var. 523 |
| 8-10 | Var. 524 |
| 10-40 | Var. 525 |
| 10-38 | Var. 526 |
| 10-36 | Var. 527 |
| 10-34 | Var. 528 |
| 10-32 | Var. 529 |
| 10-30 | Var. 530 |
| 10-28 | Var. 531 |
| 10-26 | Var. 532 |
| 10-24 | Var. 533 |
| 10-22 | Var. 534 |
| 10-20 | Var. 535 |
| 10-18 | Var. 536 |
| 10-16 | Var. 537 |
| 10-14 | Var. 538 |
| 10-12 | Var. 539 |
| 12-40 | Var. 540 |
| 12-38 | Var. 541 |
| 12-36 | Var. 542 |
| 12-34 | Var. 543 |
| 12-32 | Var. 544 |
| 12-30 | Var. 545 |
| 12-28 | Var. 546 |
| 12-26 | Var. 547 |
| 12-24 | Var. 548 |
| 12-22 | Var. 549 |
| 12-20 | Var. 550 |
| 12-18 | Var. 551 |
| 12-16 | Var. 552 |
| 12-14 | Var. 553 |
| 14-40 | Var. 554 |
| 14-38 | Var. 555 |
| 14-36 | Var. 556 |
| 14-34 | Var. 557 |
| 14-32 | Var. 558 |
| 14-30 | Var. 559 |
| 14-28 | Var. 560 |
| 14-26 | Var. 561 |
| 14-24 | Var. 562 |
| 14-22 | Var. 563 |
| 14-20 | Var. 564 |
| 14-18 | Var. 565 |
| 14-16 | Var. 566 |
| 16-40 | Var. 567 |
| 16-38 | Var. 568 |
| 16-36 | Var. 569 |
| 16-34 | Var. 570 |
| 16-32 | Var. 571 |
| 16-30 | Var. 572 |
| 16-28 | Var. 573 |
| 16-26 | Var. 574 |
| 16-24 | Var. 575 |
| 16-22 | Var. 576 |
| 16-20 | Var. 577 |
| 16-18 | Var. 578 |
| 18-40 | Var. 579 |
| 18-38 | Var. 580 |
| 18-36 | Var. 581 |
| 18-34 | Var. 582 |
| 18-32 | Var. 583 |
| 18-30 | Var. 584 |
| 18-28 | Var. 585 |
| 18-26 | Var. 586 |
| 18-24 | Var. 587 |
| 18-22 | Var. 588 |
| 18-20 | Var. 589 |
| 20-40 | Var. 590 |
| 20-38 | Var. 591 |
| 20-36 | Var. 592 |
| 20-34 | Var. 593 |
| 20-32 | Var. 594 |
| 20-30 | Var. 595 |
| 20-28 | Var. 596 |
| 20-26 | Var. 597 |
| 20-24 | Var. 598 |
| 20-22 | Var. 599 |
| 22-40 | Var. 600 |
| 22-38 | Var. 601 |
| 22-36 | Var. 602 |
| 22-34 | Var. 603 |
| 22-32 | Var. 604 |
| 22-30 | Var. 605 |
| 22-28 | Var. 606 |
| 22-26 | Var. 607 |
| 22-24 | Var. 608 |
| 24-40 | Var. 609 |
| 24-38 | Var. 610 |
| 24-36 | Var. 611 |
| 24-34 | Var. 612 |
| 24-32 | Var. 613 |
| 24-30 | Var. 614 |
| 24-28 | Var. 615 |
| 24-26 | Var. 616 |
| 26-40 | Var. 617 |
| 26-38 | Var. 618 |
| 26-36 | Var. 619 |
| 26-34 | Var. 620 |
| 26-32 | Var. 621 |
| 26-30 | Var. 622 |
| 26-28 | Var. 623 |
| 28-40 | Var. 624 |
| 28-38 | Var. 625 |
| 28-36 | Var. 626 |
| 28-34 | Var. 627 |
| 28-32 | Var. 628 |
| 28-30 | Var. 629 |
| 30-40 | Var. 630 |
| 30-38 | Var. 631 |
| 30-36 | Var. 632 |
| 30-34 | Var. 633 |
| 30-32 | Var. 634 |
| 32-40 | Var. 635 |
| 32-38 | Var. 636 |
| 32-36 | Var. 637 |
| 32-34 | Var. 638 |
| 34-40 | Var. 639 |
| 36-38 | Var. 640 |
| 38-40 | Var. 641 |

Var. = Variation

In some embodiments, the rVWF composition prepared by the methods provided herein can be characterized according to the percentage of rVWF molecules that are present in a particular higher order rVWF multimer or larger multimer. For example, in one embodiment, at least 20% of rVWF molecules in a rVWF composition used in the methods described herein are present in an oligomeric complex of at least 10 subunits. In another embodiment, at least 20% of rVWF molecules in a rVWF composition used in the methods described herein are present in an oligomeric complex of at least 12 subunits. In yet other embodiments, a rVWF composition used in the methods provided herein has a minimal percentage (e.g., has at least X %) of rVWF molecules present in a particular higher-order rVWF multimer or larger multimer (e.g., a multimer of at least Y subunits) according to any one of variations 134 to 457 found in Table 5 to Table 7.

TABLE 5
Exemplary embodiments for the percentage of rVWF molecules that are
present in a particular higher order rVWF multimer or larger multimer
found in the compositions and used in the methods provided herein.

| | | Minimal Number of Subunits in rVWF Multimer | | | | | |
|---|---|---|---|---|---|---|---|
| | | 6 | 8 | 10 | 12 | 14 | 16 |
| Minimal | 10% | Var. 134 | Var. 152 | Var. 170 | Var. 188 | Var. 206 | Var. 224 |
| Percentage | 15% | Var. 135 | Var. 153 | Var. 171 | Var. 189 | Var. 207 | Var. 225 |
| of rVWF | 20% | Var. 136 | Var. 154 | Var. 172 | Var. 190 | Var. 208 | Var. 226 |
| [symbol] | 25% | Var. 137 | Var. 155 | Var. 173 | Var. 191 | Var. 209 | Var. 227 |
| | 30% | Var. 138 | Var. 156 | Var. 174 | Var. 192 | Var. 210 | Var. 228 |
| | 35% | Var. 139 | Var. 157 | Var. 175 | Var. 193 | Var. 211 | Var. 229 |
| | 40% | Var. 140 | Var. 158 | Var. 176 | Var. 194 | Var. 212 | Var. 230 |
| | 45% | Var. 141 | Var. 159 | Var. 177 | Var. 195 | Var. 213 | Var. 231 |
| | 50% | Var. 142 | Var. 160 | Var. 178 | Var. 196 | Var. 214 | Var. 232 |
| | 55% | Var. 143 | Var. 161 | Var. 179 | Var. 197 | Var. 215 | Var. 233 |
| | 60% | Var. 144 | Var. 162 | Var. 180 | Var. 198 | Var. 216 | Var. 234 |
| | 65% | Var. 145 | Var. 163 | Var. 181 | Var. 199 | Var. 217 | Var. 235 |
| | 70% | Var. 146 | Var. 164 | Var. 182 | Var. 200 | Var. 218 | Var. 236 |
| | 75% | Var. 147 | Var. 165 | Var. 183 | Var. 201 | Var. 219 | Var. 237 |
| | 80% | Var. 148 | Var. 166 | Var. 184 | Var. 202 | Var. 220 | Var. 238 |
| | 85% | Var. 149 | Var. 167 | Var. 185 | Var. 203 | Var. 221 | Var. 239 |
| | 90% | Var. 150 | Var. 168 | Var. 186 | Var. 204 | Var. 222 | Var. 240 |
| | 95% | Var. 151 | Var. 169 | Var. 187 | Var. 205 | Var. 223 | Var. 241 |

Var. = Variation [symbol]
indicates data missing or illegible when filed

TABLE 6
Exemplary embodiments for the percentage of rVWF molecules that are
present in a particular higher order rVWF multimer or larger multimer
found in the compositions and used in the methods provided herein.

| | | Minimal Number of Subunits in rVWF Multimer | | | | | |
|---|---|---|---|---|---|---|---|
| | | 18 | 20 | 22 | 24 | 26 | 28 |
| Minimal | 10% | Var. 242 | Var. 260 | Var. 278 | Var. 296 | Var. 314 | Var. 332 |
| Percentage | 15% | Var. 243 | Var. 261 | Var. 279 | Var. 297 | Var. 315 | Var. 333 |
| of rVWF | 20% | Var. 244 | Var. 262 | Var. 280 | Var. 298 | Var. 316 | Var. 334 |
| Molecules | 25% | Var. 245 | Var. 263 | Var. 281 | Var. 299 | Var. 317 | Var. 335 |
| | 30% | Var. 246 | Var. 264 | Var. 282 | Var. 300 | Var. 318 | Var. 336 |
| | 35% | Var. 247 | Var. 265 | Var. 283 | Var. 301 | Var. 319 | Var. 337 |
| | 40% | Var. 248 | Var. 266 | Var. 284 | Var. 302 | Var. 320 | Var. 338 |
| | 45% | Var. 249 | Var. 267 | Var. 285 | Var. 303 | Var. 321 | Var. 339 |
| | 50% | Var. 250 | Var. 268 | Var. 286 | Var. 304 | Var. 322 | Var. 340 |
| | 55% | Var. 251 | Var. 269 | Var. 287 | Var. 305 | Var. 323 | Var. 341 |
| | 60% | Var. 252 | Var. 270 | Var. 288 | Var. 306 | Var. 324 | Var. 342 |
| | 65% | Var. 253 | Var. 271 | Var. 289 | Var. 307 | Var. 325 | Var. 343 |
| | 70% | Var. 254 | Var. 272 | Var. 290 | Var. 308 | Var. 326 | Var. 344 |
| | 75% | Var. 255 | Var. 273 | Var. 291 | Var. 309 | Var. 327 | Var. 345 |
| | 80% | Var. 256 | Var. 274 | Var. 292 | Var. 310 | Var. 328 | Var. 346 |
| | 85% | Var. 257 | Var. 275 | Var. 293 | Var. 311 | Var. 329 | Var. 347 |
| | 90% | Var. 258 | Var. 276 | Var. 294 | Var. 312 | Var. 330 | Var. 348 |
| | 95% | Var. 259 | Var. 277 | Var. 295 | Var. 313 | Var. 331 | Var. 349 |

Var. = Variation

TABLE 7
Exemplary embodiments for the percentage of rVWF molecules that are
present in a particular higher order rVWF multimer or larger multimer
found in the compositions and used in the methods provided herein.

| | | Minimal Number of Subunits in rVWF Multimer | | | | | |
|---|---|---|---|---|---|---|---|
| | | 30 | 32 | 34 | 36 | 38 | 40 |
| Minimal | 10% | Var. 350 | Var. 368 | Var. 386 | Var. 404 | Var. 422 | Var. 440 |
| [symbol] | 15% | Var. 351 | Var. 369 | Var. 387 | Var. 405 | Var. 423 | Var. 441 |
| | 20% | Var. 352 | Var. 370 | Var. 388 | Var. 406 | Var. 424 | Var. 442 |
| | 25% | Var. 353 | Var. 371 | Var. 389 | Var. 407 | Var. 425 | Var. 443 |
| | 30% | Var. 354 | Var. 372 | Var. 390 | Var. 408 | Var. 426 | Var. 444 |
| | 35% | Var. 355 | Var. 373 | Var. 391 | Var. 409 | Var. 427 | Var. 445 |
| | 40% | Var. 356 | Var. 374 | Var. 392 | Var. 410 | Var. 428 | Var. 446 |
| | 45% | Var. 357 | Var. 375 | Var. 393 | Var. 411 | Var. 429 | Var. 447 |
| | 50% | Var. 358 | Var. 376 | Var. 394 | Var. 412 | Var. 430 | Var. 448 |
| | 55% | Var. 359 | Var. 377 | Var. 395 | Var. 413 | Var. 431 | Var. 449 |
| | 60% | Var. 360 | Var. 378 | Var. 396 | Var. 414 | Var. 432 | Var. 450 |
| | 65% | Var. 361 | Var. 379 | Var. 397 | Var. 415 | Var. 433 | Var. 451 |
| | 70% | Var. 362 | Var. 380 | Var. 398 | Var. 416 | Var. 434 | Var. 452 |
| | 75% | Var. 363 | Var. 381 | Var. 399 | Var. 417 | Var. 435 | Var. 453 |
| | 80% | Var. 364 | Var. 382 | Var. 400 | Var. 418 | Var. 436 | Var. 454 |
| | 85% | Var. 365 | Var. 383 | Var. 401 | Var. 419 | Var. 437 | Var. 455 |
| | 90% | Var. 366 | Var. 384 | Var. 402 | Var. 420 | Var. 438 | Var. 456 |
| | 95% | Var. 367 | Var. 385 | Var. 403 | Var. 421 | Var. 439 | Var. 457 |

Var. = Variation [symbol]
indicates data missing or illegible when filed

In accordance with the above, the rVWF comprises a significant percentage of high molecular weight (HMW) rVWF multimers. In further embodiments, the HMW rVWF multimer composition comprises at least 10%-80% rVWF decamers or higher order multimers. In further embodiments, the composition comprises about 10-95%, 20-90%, 30-85%, 40-80%, 50-75%, 60-70% decamers or higher order multimers. In further embodiments, the HMW rVWF multimer composition comprises at least about 10%, 20%, 30%, 40%, 50%, 60%, 70%, 80%, 90% decamers or higher order multimers.

Assessment of the number and percentage of rVWF multimers can be conducted using methods known in the art, including without limitation methods using electrophoresis and size exclusion chromatography methods to separate rVWF multimers by size, for example as discussed by Cumming et al, (J Clin Pathol. 1993 May; 46(5): 470-473, which is hereby incorporated by reference in its entirety for all purposes and in particular for all teachings related to assessment of rVWF multimers). Such techniques may further include immunoblotting techniques (such as Western Blot), in which the gel is immunoblotted with a radiolabelled antibody against VWF followed by chemiluminescent detection (see for example Wen et al., (1993), J. Clin. Lab. Anal., 7: 317-323, which is hereby incorporated by reference in its entirety for all purposes and in particular for all teachings related to assessment of rVWF multimers). Further assays for VWF include VWF:Antigen (VWF:Ag), VWF:Ristocetin Cofactor (VWF:RCof), and VWF:Collagen Binding Activity assay (VWF:CBA), which are often used for diagnosis and classification of Von Willebrand Disease. (see for example Favaloro et al., Pathology, 1997, 29(4): 341-456, which is hereby incorporated by reference in its entirety for all purposes and in particular for all teachings related to assays for VWF).

In some embodiments, the ratio of rFVIII procoagulant activity (IU rFVIII:C) to rVWF Ristocetin cofactor activity (IU rVWF:RCo) for the rVWF prepared according to the methods of the present invention is between 3:1 and 1:5. In further embodiments, the ratio is between 2:1 and 1:4. In still further embodiments, the ratio is between 5:2 and 1:4. In further embodiments, the ratio is between 3:2 and 1:3. In still further embodiments, the ratio is about 1:1, 1:2, 1:3, 1:4, 1:5, 2:1, 2:3, 2:4, 2:5, 3:1, 3:2, 3:4, or 3:5. In further embodiments, the ratio is between 1:1 and 1:2. In yet further embodiments, the ratio is 1.1:1, 1.2:1, 1.3:1, 1.4:1, 1.5:1, 1.6:1, 1.7:1, 1.8:1, 1.9:1, or 2:1. In certain embodiments, the ratio of rFVIII procoagulant activity (IU rFVIII:C) to rVWF Ristocetin cofactor activity (IU rVWF:RCo) in a composition useful for a method described herein is selected from variations 1988 to 2140 found in Table 8.

TABLE 8
Exemplary embodiments for the ratio of rFVIII procoagulant activity
(IU rFVIII:C) to rVWF Ristocetin cofactor activity (IU rVWF:RCo)
in compositions and used in methods provided herein.

| (IU rFVIII:C) to | |
|---|---|
| (IU rVWF:RCo) | |
| 4:1 | Var. 1988 |
| 3:1 | Var. 1989 |
| 2:1 | Var. 1990 |
| 3:2 | Var. 1991 |
| 4:3 | Var. 1992 |
| 1:1 | Var. 1993 |
| 5:6 | Var. 1994 |
| 4:5 | Var. 1995 |
| 3:4 | Var. 1996 |
| 2:3 | Var. 1997 |
| 3:5 | Var. 1998 |
| 1:2 | Var. 1999 |
| 2:5 | Var. 2000 |
| 1:3 | Var. 2001 |
| 1:4 | Var. 2002 |
| 1:5 | Var. 2003 |
| 1:6 | Var. 2004 |
| 4:1-1:6 | Var. 2005 |
| 4:1-1:5 | Var. 2006 |
| 4:1-1:4 | Var. 2007 |
| 4:1-1:3 | Var. 2008 |
| 4:1-2:5 | Var. 2009 |
| 4:1-1:2 | Var. 2010 |
| 4:1-3:5 | Var. 2011 |
| 4:1-2:3 | Var. 2012 |
| 4:1-3:4 | Var. 2013 |
| 4:1-4:5 | Var. 2014 |
| 4:1-5:6 | Var. 2015 |
| 4:1-1:1 | Var. 2016 |
| 4:1-4:3 | Var. 2017 |
| 4:1-3:2 | Var. 2018 |
| 4:1-2:1 | Var. 2019 |
| 4:1-3:1 | Var. 2020 |
| 3:1-1:6 | Var. 2021 |
| 3:1-1:5 | Var. 2022 |
| 3:1-1:4 | Var. 2023 |
| 3:1-1:3 | Var. 2024 |
| 3:1-2:5 | Var. 2025 |
| 3:1-1:2 | Var. 2026 |
| 3:1-3:5 | Var. 2027 |
| 3:1-2:3 | Var. 2028 |
| 3:1-3:4 | Var. 2029 |
| 3:1-4:5 | Var. 2030 |
| 3:1-5:6 | Var. 2031 |
| 3:1-1:1 | Var. 2032 |
| 3:1-4:3 | Var. 2033 |
| 3:1-3:2 | Var. 2034 |
| 3:1-2:1 | Var. 2035 |
| 2:1-1:6 | Var. 2036 |
| 2:1-1:5 | Var. 2037 |
| 2:1-1:4 | Var. 2038 |
| 2:1-1:3 | Var. 2039 |
| 2:1-2:5 | Var. 2040 |
| 2:1-1:2 | Var. 2041 |
| 2:1-3:5 | Var. 2042 |
| 2:1-2:3 | Var. 2043 |
| 2:1-3:4 | Var. 2044 |
| 2:1-4:5 | Var. 2045 |
| 2:1-5:6 | Var. 2046 |
| 2:1-1:1 | Var. 2047 |
| 2:1-4:3 | Var. 2048 |
| 2:1-3:2 | Var. 2049 |
| 3:2-1:6 | Var. 2050 |
| 3:2-1:5 | Var. 2051 |
| 3:2-1:4 | Var. 2052 |
| 3:2-1:3 | Var. 2053 |
| 3:2-2:5 | Var. 2054 |
| 3:2-1:2 | Var. 2055 |
| 3:2-3:5 | Var. 2056 |
| 3:2-2:3 | Var. 2057 |
| 3:2-3:4 | Var. 2058 |
| 3:2-4:5 | Var. 2059 |
| 3:2-5:6 | Var. 2060 |
| 3:2-1:1 | Var. 2061 |
| 3:2-4:3 | Var. 2062 |
| 4:3-1:6 | Var. 2063 |
| 4:3-1:5 | Var. 2064 |
| 4:3-1:4 | Var. 2065 |
| 4:3-1:3 | Var. 2066 |
| 4:3-2:5 | Var. 2067 |
| 4:3-1:2 | Var. 2068 |
| 4:3-3:5 | Var. 2069 |
| 4:3-2:3 | Var. 2070 |
| 4:3-3:4 | Var. 2071 |
| 4:3-4:5 | Var. 2072 |
| 4:3-5:6 | Var. 2073 |
| 4:3-1:1 | Var. 2074 |
| 1:1-1:6 | Var. 2075 |
| 1:1-1:5 | Var. 2076 |
| 1:1-1:4 | Var. 2077 |
| 1:1-1:3 | Var. 2078 |
| 1:1-2:5 | Var. 2079 |
| 1:1-1:2 | Var. 2080 |
| 1:1-3:5 | Var. 2081 |
| 1:1-2:3 | Var. 2082 |
| 1:1-3:4 | Var. 2083 |
| 1:1-4:5 | Var. 2084 |
| 1:1-5:6 | Var. 2085 |
| 5:6-1:6 | Var. 2086 |
| 5:6-1:5 | Var. 2087 |
| 5:6-1:4 | Var. 2088 |
| 5:6-1:3 | Var. 2089 |
| 5:6-2:5 | Var. 2090 |
| 5:6-1:2 | Var. 2091 |
| 5:6-3:5 | Var. 2092 |
| 5:6-2:3 | Var. 2093 |
| 5:6-3:4 | Var. 2094 |
| 5:6-4:5 | Var. 2095 |
| 4:5-1:6 | Var. 2096 |
| 4:5-1:5 | Var. 2097 |
| 4:5-1:4 | Var. 2098 |
| 4:5-1:3 | Var. 2099 |
| 4:5-2:5 | Var. 2100 |
| 4:5-1:2 | Var. 2101 |
| 4:5-3:5 | Var. 2102 |
| 4:5-2:3 | Var. 2103 |
| 4:5-3:4 | Var. 2104 |
| 3:4-1:6 | Var. 2105 |
| 3:4-1:5 | Var. 2106 |
| 3:4-1:4 | Var. 2107 |
| 3:4-1:3 | Var. 2108 |
| 3:4-2:5 | Var. 2109 |
| 3:4-1:2 | Var. 2110 |
| 3:4-3:5 | Var. 2111 |
| 3:4-2:3 | Var. 2112 |
| 2:3-1:6 | Var. 2113 |
| 2:3-1:5 | Var. 2114 |
| 2:3-1:4 | Var. 2115 |
| 2:3-1:3 | Var. 2116 |
| 2:3-2:5 | Var. 2117 |
| 2:3-1:2 | Var. 2118 |
| 2:3-3:5 | Var. 2119 |
| 3:5-1:6 | Var. 2120 |
| 3:5-1:5 | Var. 2121 |
| 3:5-1:4 | Var. 2122 |
| 3:5-1:3 | Var. 2123 |
| 3:5-2:5 | Var. 2124 |
| 3:5-1:2 | Var. 2125 |
| 1:2-1:6 | Var. 2126 |
| 1:2-1:5 | Var. 2127 |
| 1:2-1:4 | Var. 2128 |
| 1:2-1:3 | Var. 2129 |
| 1:2-2:5 | Var. 2130 |
| 2:5-1:6 | Var. 2131 |
| 2:5-1:5 | Var. 2132 |
| 2:5-1:4 | Var. 2133 |
| 2:5-1:3 | Var. 2134 |
| 1:3-1:6 | Var. 2135 |
| 1:3-1:5 | Var. 2136 |
| 1:3-1:4 | Var. 2137 |
| 1:4-1:6 | Var. 2138 |
| 1:4-1:5 | Var. 2139 |
| 1:5-1:6 | Var. 2140 |

Var. = Variation

In further embodiments, higher order rVWF multimers of the invention are stable for about 1 to about 90 hours post-administration. In still further embodiments, the higher order rVWF multimers are stable for about 5-80, 10-70, 15-60, 20-50, 25-40, 30-35 hours post-administration. In yet further embodiments, the higher order rVWF multimers are stable for at least 3, 6, 12, 18, 24, 36, 48, 72 hours post-administration. In certain embodiments the stability of the rVWF multimers is assessed in vitro.

In one embodiment, higher order rVWF multimers used in the compositions and methods provided herein have a half-life of at least 12 hour post administration. In another embodiment, the higher order rVWF multimers have a half-life of at least 24 hour post administration. In yet other embodiments, the higher order rVWF multimers have a half-life selected from variations 642 to 1045 found in Table 9.

TABLE 9
Exemplary embodiments for the half-life of higher
order rVWF multimers found in the compositions
prepared by the methods provided herein.

| Hours | |
|---|---|
| at least 1 | Var. 642 |
| at least 2 | Var. 643 |
| at least 3 | Var. 644 |
| at least 4 | Var. 645 |
| at least 5 | Var. 646 |
| at least 6 | Var. 647 |
| at least 7 | Var. 648 |
| at least 8 | Var. 649 |
| at least 9 | Var. 650 |
| at least 10 | Var. 651 |
| at least 11 | Var. 652 |
| at least 12 | Var. 653 |
| at least 14 | Var. 654 |
| at least 16 | Var. 655 |
| at least 18 | Var. 656 |
| at least 20 | Var. 657 |
| at least 22 | Var. 658 |
| at least 24 | Var. 659 |
| at least 27 | Var. 660 |
| at least 30 | Var. 661 |
| at least 33 | Var. 662 |
| at least 36 | Var. 663 |
| at least 39 | Var. 664 |
| at least 42 | Var. 665 |
| at least 45 | Var. 666 |
| at least 48 | Var. 667 |
| at least 54 | Var. 668 |
| at least 60 | Var. 669 |
| at least 66 | Var. 670 |
| at least 72 | Var. 671 |
| at least 78 | Var. 672 |
| at least 84 | Var. 673 |
| at least 90 | Var. 674 |
| 2-90 | Var. 675 |
| 2-84 | Var. 676 |
| 2-78 | Var. 677 |
| 2-72 | Var. 678 |
| 2-66 | Var. 679 |
| 2-60 | Var. 680 |
| 2-54 | Var. 681 |
| 2-48 | Var. 682 |
| 2-45 | Var. 683 |
| 2-42 | Var. 684 |
| 2-39 | Var. 685 |
| 2-36 | Var. 686 |
| 2-33 | Var. 687 |
| 2-30 | Var. 688 |
| 2-27 | Var. 689 |
| 2-24 | Var. 690 |
| 2-22 | Var. 691 |
| 2-20 | Var. 692 |
| 2-18 | Var. 693 |
| 2-16 | Var. 694 |
| 2-14 | Var. 695 |
| 2-12 | Var. 696 |
| 2-10 | Var. 697 |
| 2-8 | Var. 698 |
| 2-6 | Var. 699 |
| 2-4 | Var. 700 |
| 3-90 | Var. 701 |
| 3-84 | Var. 702 |
| 3-78 | Var. 703 |
| 3-72 | Var. 704 |
| 3-66 | Var. 705 |
| 3-60 | Var. 706 |
| 3-54 | Var. 707 |
| 3-48 | Var. 708 |
| 3-45 | Var. 709 |
| 3-42 | Var. 710 |
| 3-39 | Var. 711 |
| 3-36 | Var. 712 |
| 3-33 | Var. 713 |
| 3-30 | Var. 714 |
| 3-27 | Var. 715 |
| 3-24 | Var. 716 |
| 3-22 | Var. 717 |
| 3-20 | Var. 718 |
| 3-18 | Var. 719 |
| 3-16 | Var. 720 |
| 3-14 | Var. 721 |
| 3-12 | Var. 722 |
| 3-10 | Var. 723 |
| 3-8 | Var. 724 |
| 3-6 | Var. 725 |
| 3-4 | Var. 726 |
| 4-90 | Var. 727 |
| 4-84 | Var. 728 |
| 4-78 | Var. 729 |
| 4-72 | Var. 730 |
| 4-66 | Var. 731 |
| 4-60 | Var. 732 |
| 4-54 | Var. 733 |
| 4-48 | Var. 734 |
| 4-45 | Var. 735 |
| 4-42 | Var. 736 |
| 4-39 | Var. 737 |
| 4-36 | Var. 738 |
| 4-33 | Var. 739 |
| 4-30 | Var. 740 |
| 4-27 | Var. 741 |
| 4-24 | Var. 742 |
| 4-22 | Var. 743 |
| 4-20 | Var. 744 |
| 4-18 | Var. 745 |
| 4-16 | Var. 746 |
| 4-14 | Var. 747 |
| 4-12 | Var. 748 |
| 4-10 | Var. 749 |
| 4-8 | Var. 750 |
| 4-6 | Var. 751 |
| 6-90 | Var. 752 |
| 6-84 | Var. 753 |
| 6-78 | Var. 754 |
| 6-72 | Var. 755 |
| 6-66 | Var. 756 |
| 6-60 | Var. 757 |
| 6-54 | Var. 758 |
| 6-48 | Var. 759 |
| 6-45 | Var. 760 |
| 6-42 | Var. 761 |
| 6-39 | Var. 762 |
| 6-36 | Var. 763 |
| 6-33 | Var. 764 |
| 6-30 | Var. 765 |
| 6-27 | Var. 766 |
| 6-24 | Var. 767 |
| 6-22 | Var. 768 |
| 6-20 | Var. 769 |
| 6-18 | Var. 770 |
| 6-16 | Var. 771 |
| 6-14 | Var. 772 |
| 6-12 | Var. 773 |
| 6-10 | Var. 774 |
| 6-8 | Var. 775 |
| 8-90 | Var. 776 |
| 8-84 | Var. 777 |
| 8-78 | Var. 778 |
| 8-72 | Var. 779 |
| 8-66 | Var. 780 |
| 8-60 | Var. 781 |
| 8-54 | Var. 782 |
| 8-48 | Var. 783 |
| 8-45 | Var. 784 |
| 8-42 | Var. 785 |
| 8-39 | Var. 786 |
| 8-36 | Var. 787 |
| 8-33 | Var. 788 |
| 8-30 | Var. 789 |
| 8-27 | Var. 790 |
| 8-24 | Var. 791 |
| 8-22 | Var. 792 |
| 8-20 | Var. 793 |
| 8-18 | Var. 794 |
| 8-16 | Var. 795 |
| 8-14 | Var. 796 |
| 8-12 | Var. 797 |
| 8-10 | Var. 798 |
| 10-90 | Var. 799 |
| 10-84 | Var. 800 |
| 10-78 | Var. 801 |
| 10-72 | Var. 802 |
| 10-66 | Var. 803 |
| 10-60 | Var. 804 |
| 10-54 | Var. 805 |
| 10-48 | Var. 806 |
| 10-45 | Var. 807 |
| 10-42 | Var. 808 |
| 10-39 | Var. 809 |
| 10-36 | Var. 810 |
| 10-33 | Var. 811 |
| 10-30 | Var. 812 |
| 10-27 | Var. 813 |
| 10-24 | Var. 814 |
| 10-22 | Var. 815 |
| 10-20 | Var. 816 |
| 10-18 | Var. 817 |
| 10-16 | Var. 818 |
| 10-14 | Var. 819 |
| 10-12 | Var. 820 |
| 12-90 | Var. 821 |
| 12-84 | Var. 822 |
| 12-78 | Var. 823 |
| 12-72 | Var. 824 |
| 12-66 | Var. 825 |
| 12-60 | Var. 826 |
| 12-54 | Var. 827 |
| 12-48 | Var. 828 |
| 12-45 | Var. 829 |
| 12-42 | Var. 830 |
| 12-39 | Var. 831 |
| 12-36 | Var. 832 |
| 12-33 | Var. 833 |
| 12-30 | Var. 834 |
| 12-27 | Var. 835 |
| 12-24 | Var. 836 |
| 12-22 | Var. 837 |
| 12-20 | Var. 838 |
| 12-18 | Var. 839 |
| 12-16 | Var. 840 |
| 12-14 | Var. 841 |
| 14-90 | Var. 842 |
| 14-84 | Var. 843 |
| 14-78 | Var. 844 |
| 14-72 | Var. 845 |
| 14-66 | Var. 846 |
| 14-60 | Var. 847 |
| 14-54 | Var. 848 |
| 14-48 | Var. 849 |
| 14-45 | Var. 850 |
| 14-42 | Var. 851 |
| 14-39 | Var. 852 |
| 14-36 | Var. 853 |
| 14-33 | Var. 854 |
| 14-30 | Var. 855 |
| 14-27 | Var. 856 |
| 14-24 | Var. 857 |
| 14-22 | Var. 858 |
| 14-20 | Var. 859 |
| 14-18 | Var. 860 |
| 14-16 | Var. 861 |
| 16-90 | Var. 862 |
| 16-84 | Var. 863 |
| 16-78 | Var. 864 |
| 16-72 | Var. 865 |
| 16-66 | Var. 866 |
| 16-60 | Var. 867 |
| 16-54 | Var. 868 |
| 16-48 | Var. 869 |
| 16-45 | Var. 870 |
| 16-42 | Var. 871 |
| 16-39 | Var. 872 |
| 16-36 | Var. 873 |
| 16-33 | Var. 874 |
| 16-30 | Var. 875 |
| 16-27 | Var. 876 |
| 16-24 | Var. 877 |
| 16-22 | Var. 878 |
| 16-20 | Var. 879 |
| 16-18 | Var. 880 |
| 18-90 | Var. 881 |
| 18-84 | Var. 882 |
| 18-78 | Var. 883 |
| 18-72 | Var. 884 |
| 18-66 | Var. 885 |
| 18-60 | Var. 886 |
| 18-54 | Var. 887 |
| 18-48 | Var. 888 |
| 18-45 | Var. 889 |
| 18-42 | Var. 890 |
| 18-39 | Var. 891 |
| 18-36 | Var. 892 |
| 18-33 | Var. 893 |
| 18-30 | Var. 894 |
| 18-27 | Var. 895 |
| 18-24 | Var. 896 |
| 18-22 | Var. 897 |
| 18-20 | Var. 898 |
| 20-90 | Var. 899 |
| 20-84 | Var. 900 |
| 20-78 | Var. 901 |
| 20-72 | Var. 902 |
| 20-66 | Var. 903 |
| 20-60 | Var. 904 |
| 20-54 | Var. 905 |
| 20-48 | Var. 906 |
| 20-45 | Var. 907 |
| 20-42 | Var. 908 |
| 20-39 | Var. 909 |
| 20-36 | Var. 910 |
| 20-33 | Var. 911 |
| 20-30 | Var. 912 |
| 20-27 | Var. 913 |
| 20-24 | Var. 914 |
| 20-22 | Var. 915 |
| 22-90 | Var. 916 |
| 22-84 | Var. 917 |
| 22-78 | Var. 918 |
| 22-72 | Var. 919 |
| 22-66 | Var. 920 |
| 22-60 | Var. 921 |
| 22-54 | Var. 922 |
| 22-48 | Var. 923 |
| 22-45 | Var. 924 |
| 22-42 | Var. 925 |
| 22-39 | Var. 926 |
| 22-36 | Var. 927 |
| 22-33 | Var. 928 |
| 22-30 | Var. 929 |
| 22-27 | Var. 930 |
| 22-24 | Var. 931 |
| 24-90 | Var. 932 |
| 24-84 | Var. 933 |
| 24-78 | Var. 934 |
| 24-72 | Var. 935 |
| 24-66 | Var. 936 |
| 24-60 | Var. 937 |
| 24-54 | Var. 938 |
| 24-48 | Var. 939 |
| 24-45 | Var. 940 |
| 24-42 | Var. 941 |
| 24-39 | Var. 942 |
| 24-36 | Var. 943 |
| 24-33 | Var. 944 |
| 24-30 | Var. 945 |
| 24-27 | Var. 946 |
| 27-90 | Var. 947 |
| 27-84 | Var. 948 |
| 27-78 | Var. 949 |
| 27-72 | Var. 950 |
| 27-66 | Var. 951 |
| 27-60 | Var. 952 |
| 27-54 | Var. 953 |
| 27-48 | Var. 954 |
| 30-90 | Var. 955 |
| 30-84 | Var. 956 |
| 30-78 | Var. 957 |
| 30-72 | Var. 958 |
| 30-66 | Var. 959 |
| 30-60 | Var. 960 |
| 30-54 | Var. 961 |
| 30-48 | Var. 962 |
| 30-45 | Var. 963 |
| 30-42 | Var. 964 |
| 30-39 | Var. 965 |
| 30-36 | Var. 966 |
| 30-33 | Var. 967 |
| 33-90 | Var. 968 |
| 33-84 | Var. 969 |
| 33-78 | Var. 970 |
| 33-72 | Var. 971 |
| 33-66 | Var. 972 |
| 33-60 | Var. 973 |
| 33-54 | Var. 974 |
| 33-48 | Var. 975 |
| 33-45 | Var. 976 |
| 33-42 | Var. 977 |
| 33-29 | Var. 978 |
| 33-36 | Var. 979 |
| 36-90 | Var. 980 |
| 36-84 | Var. 981 |
| 36-78 | Var. 982 |
| 36-72 | Var. 983 |
| 36-66 | Var. 984 |
| 36-60 | Var. 985 |
| 36-54 | Var. 986 |
| 36-48 | Var. 987 |
| 36-45 | Var. 988 |
| 36-42 | Var. 989 |
| 36-39 | Var. 990 |
| 39-90 | Var. 991 |
| 39-84 | Var. 992 |
| 39-78 | Var. 993 |
| 39-72 | Var. 994 |
| 39-66 | Var. 995 |
| 39-60 | Var. 996 |
| 39-54 | Var. 997 |
| 39-48 | Var. 998 |
| 39-45 | Var. 999 |
| 39-42 | Var. 1000 |
| 42-90 | Var. 1001 |
| 42-84 | Var. 1002 |
| 42-78 | Var. 1003 |
| 42-72 | Var. 1004 |
| 42-66 | Var. 1005 |
| 42-60 | Var. 1006 |
| 42-54 | Var. 1007 |
| 42-48 | Var. 1008 |
| 42-45 | Var. 1009 |
| 45-90 | Var. 1010 |
| 45-84 | Var. 1011 |
| 45-78 | Var. 1012 |
| 45-72 | Var. 1013 |
| 45-66 | Var. 1014 |
| 45-60 | Var. 1015 |
| 45-54 | Var. 1016 |
| 45-48 | Var. 1017 |
| 48-90 | Var. 1018 |
| 48-84 | Var. 1019 |
| 48-78 | Var. 1020 |
| 48-72 | Var. 1021 |
| 48-66 | Var. 1022 |
| 48-60 | Var. 1023 |
| 48-54 | Var. 1024 |
| 54-90 | Var. 1025 |
| 54-84 | Var. 1026 |
| 54-78 | Var. 1027 |
| 54-72 | Var. 1028 |
| 54-66 | Var. 1029 |
| 54-60 | Var. 1030 |
| 60-90 | Var. 1031 |
| 60-84 | Var. 1032 |
| 60-78 | Var. 1033 |
| 60-72 | Var. 1034 |
| 60-66 | Var. 1035 |
| 66-90 | Var. 1036 |
| 66-84 | Var. 1037 |
| 66-78 | Var. 1038 |
| 66-72 | Var. 1039 |
| 72-90 | Var. 1040 |
| 72-84 | Var. 1041 |
| 72-78 | Var. 1042 |
| 78-90 | Var. 1043 |
| 78-84 | Var. 1044 |
| 84-90 | Var. 1045 |

Var. = Variation

In some embodiments, the pro-VWF and/or purified rVWF purified in accordance with the present invention is not modified with any conjugation, post-translation or covalent modifications. In particular embodiments, the pro-VWF and/or purified rVWF of the present invention is not modified with a water soluble polymer, including without limitation, a polyethylene glycol (PEG), a polypropylene glycol, a polyoxyalkylene, a polysialic acid, hydroxyl ethyl starch, a poly-carbohydrate moiety, and the like.

In some embodiments, the pro-VWF and/or purified rVWF purified in accordance with the present invention is modified through conjugation, post-translation modification, or covalent modification, including modifications of the N- or C-terminal residues as well as modifications of selected side chains, for example, at free sulfhydryl-groups, primary amines, and hydroxyl-groups. In one embodiment, a water soluble polymer is linked to the protein (directly or via a linker) by a lysine group or other primary amine. In some embodiments, the pro-VWF and/or purified rVWF of the present invention may be modified by conjugation of a water soluble polymer, including without limitation, a polyethylene glycol (PEG), a polypropylene glycol, a polyoxyalkylene, a polysialic acid, hydroxyl ethyl starch, a poly-carbohydrate moiety, and the like.

Water soluble polymers that may be used to modify the pro-VWF and/or purified rVWF include linear and branched structures. The conjugated polymers may be attached directly to the coagulation proteins of the invention, or alternatively may be attached through a linking moiety. Non-limiting examples of protein conjugation with water soluble polymers can be found in U.S. Pat. Nos. 4,640,835; 4,496,689; 4,301,144; 4,670,417; 4,791,192, and 4,179,337, as well as in Abuchowski and Davis “Enzymes as Drugs,” Holcenberg and Roberts, Eds., pp. 367 383, John Wiley and Sons, New York (1981), and Hermanson G., Bioconjugate Techniques 2nd Ed., Academic Press, Inc. 2008.

Protein conjugation may be performed by a number of well-known techniques in the art, for example, see Hermanson G., Bioconjugate Techniques 2nd Ed., Academic Press, Inc. 2008. Examples include linkage through the peptide bond between a carboxyl group on one of either the coagulation protein or water-soluble polymer moiety and an amine group of the other, or an ester linkage between a carboxyl group of one and a hydroxyl group of the other. Another linkage by which a coagulation protein of the invention could be conjugated to a water-soluble polymer compound is via a Schiff base, between a free amino group on the polymer moiety being reacted with an aldehyde group formed at the non-reducing end of the polymer by periodate oxidation (Jennings and Lugowski, J. Immunol. 1981; 127:1011-8; Femandes and Gregonradis, Biochim Biophys Acta. 1997; 1341; 26-34). The generated Schiff Base can be stabilized by specific reduction with NaCNBH₃ to form a secondary amine. An alternative approach is the generation of terminal free amino groups on the polymer by reductive amination with NH₄Cl after prior oxidation. Bifunctional reagents can be used for linking two amino or two hydroxyl groups. For example, a polymer containing an amino group can be coupled to an amino group of the coagulation protein with reagents like BS3 (Bis(sulfosuccinimidyl)suberate/Pierce, Rockford, Ill.). In addition, heterobifunctional cross linking reagents like Sulfo-EMCS (N-c-Maleimidocaproyloxy) sulfosuccinimide ester/Pierce) can be used for instance to link amine and thiol groups. In other embodiments, an aldehyde reactive group, such as PEG alkoxide plus diethyl acetal of bromoacetaldehyde; PEG plus DMSO and acetic anhydride, and PEG chloride plus the phenoxide of 4-hydroxybenzaldehyde, succinimidyl active esters, activated dithiocarbonate PEG, 2,4,5-trichlorophenylcloroformate and P-nitrophenylcloroformate activated PEG, may be used in the conjugation of a coagulation protein.

Another method for measuring the biological activity of VWF is the collagen binding assay, which is based on ELISA technology (Brown and Bosak, Thromb. Res., 1986, 43:303-311; Favaloro, Thromb. Haemost., 2000, 83 127-135). A microtiter plate is coated with type I or III collagen. Then the VWF is bound to the collagen surface and subsequently detected with an enzyme-labeled polyclonal antibody. The last step is a substrate reaction, which can be photometrically monitored with an ELISA reader.

Immunological assays of von Willebrand factors (VWF:Ag) are immunoassays that measure the concentration of the VWF protein in plasma. They give no indication as to VWF function. A number of methods exist for measuring VWF:Ag and these include both enzyme-linked immunosorbent assay (ELISA) or automated latex immunoassays (LIA.) Many laboratories now use a fully automated latex immunoassay. Historically laboratories used a variety of techniques including Laurell electroimmunoassay ‘Laurell Rockets’ but these are rarely used in most labs today.

III. Kits

As an additional aspect, the invention includes kits which comprise one or more lyophilized compositions packaged in a manner which facilitates their use for administration to subjects. In one embodiment, such a kit includes pharmaceutical formulation described herein (e.g., a composition comprising a therapeutic protein or peptide), packaged in a container such as a sealed bottle or vessel, with a label affixed to the container or included in the package that describes use of the compound or composition in practicing the method. In one embodiment, the pharmaceutical formulation is packaged in the container such that the amount of headspace in the container (e.g., the amount of air between the liquid formulation and the top of the container) is very small. Preferably, the amount of headspace is negligible (e.g., almost none). In one embodiment, the kit contains a first container having a therapeutic protein or peptide composition and a second container having a physiologically acceptable reconstitution solution for the composition. In one aspect, the pharmaceutical formulation is packaged in a unit dosage form. The kit may further include a device suitable for administering the pharmaceutical formulation according to a specific route of administration. Preferably, the kit contains a label that describes use of the pharmaceutical formulations.

IV. rVWF for Methods of Prophylactic Treatment of Spontaneous Bleeding in Patients with Severe VWD

One of the advantages of administering rVWF to subjects with severe VWD for prophylactic treatment of spontaneous bleeding episodes is that the higher specific activity of rVWF as compared to pdVWF allows for flexibility in the amount of rVWF administered and the number of times the subject is re-dosed. As will be appreciated and as is discussed in further detail herein, the co-administered FVIII may be recombinant or plasma derived.

Single or multiple administrations of rVWF are carried out with the dose levels and pattern being selected by the treating physician. For the prevention or treatment of disease, the appropriate dosage depends on the type of disease to be treated (e.g., von Willebrand disease), the severity and course of the disease, whether drug is administered for preventive or therapeutic purposes, previous therapy, the patient's clinical history and response to the drug, and the discretion of the attending physician.

In some aspects, rVWF is administered prophylactically to a subject at a dose ranging from 40-80 IU/kg, e.g., 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 40-80, 45-80, 50-80, 45-70, 45-60, 45-55, 45-50, 50-60, 55-60, 60-65, 55-65, 60-70, 65-70, 60-75, 70-80, or 75-80 IU/kg. In some embodiments, the dose ranges from 40 IU/kg to 60 IU/kg. In some embodiments, the dose ranges from 45 IU/kg to 55 IU/kg. In some embodiments, the dose is about 40 IU/kg, about 50 IU/kg, or about 60 IU/kg. In some embodiments, the dose is about 50 IU/kg. In some embodiments, the dose is about 80 IU/kg. In some embodiments, rVWF is administered once a week, two times a week, three times a week, four times a week, five times a week, or more. In some embodiments, rVWF is administered twice a week. In some embodiments, rVWF is administered twice a week. In some embodiments, rVWF is administered twice a week as a dose ranging from 40 IU/kg to 60 IU/kg. In some embodiments, rVWF is administered twice a week as a dose ranging from 40 IU/kg to 60 IU/kg by IV infusion.

In some embodiments, blood samples to measure vWF:Ag, vWF:RCo, vWF:CB and FVIII activity levels were taken pre-dose, at 15 minutes, 30 minutes, and 60 minutes after drug infusion, and at 3 hours, 6 hours, 12 hours, 24 hours, 28 hours, 32 hours, 48 hours, 72 hours, and 96 hours. In some embodiments, samples, including for example blood samples, can be obtained for examining vWF:RCo and FVIII activity. In some embodiments, samples for FVIII, FVIII:C, VWF:RCo, VWF:Ag, and VWF collagen-binding capacity activity are obtained prior to the prophylactic treatment with rVWF. In some embodiments, samples for examining FVIII, FVIII:C, VWF:RCo, VWF:Ag, and VWF collagen-binding capacity activity are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF. In some embodiments, samples for examining FVIII, FVIII:C, VWF:RCo, VWF:Ag, and VWF collagen-binding capacity activity are obtained 25 to 31 days after prophylactic treatment with rVWF. In some embodiments, samples for FVIII, FVIII:C, VWF:RCo, VWF:Ag, and VWF collagen-binding capacity activity are obtained after a bleeding episode and in such embodiments, a sample is take prior to rVWF administration, 2 hours after administrator and then every 12-24 hours until resolution of the bleeding event. In some embodiments, FVIII, FVIII:C, VWF:RCo, VWF:Ag, and VWF collagen-binding capacity activity versus time profiles are determined based on the samples. In some embodiments, FVIII:C is also measured by a 1-stage clotting assay versus time profiles.

In some embodiments, rVWF is administered to the subject at a range of 40-80 IU/kg, e.g., 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 40-80, 45-80, 50-80, 45-70, 45-60, 45-55, 45-50, 50-60, 55-60, 60-65, 55-65, 60-70, 65-70, 60-75, 70-80, or 75-80 IU/kg as an initial (first) administration. In some embodiments, rVWF is administered to the subject at a range of 40-80 IU/kg, e.g., 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 40-80, 45-80, 50-80, 45-70, 45-60, 45-55, 45-50, 50-60, 55-60, 60-65, 55-65, 60-70, 65-70, 60-75, 70-80, or 75-80 IU/kg as a second administration. In some embodiments, rVWF is administered to the subject at a range of 40-80 IU/kg, e.g., 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 40-80, 45-80, 50-80, 45-70, 45-60, 45-55, 45-50, 50-60, 55-60, 60-65, 55-65, 60-70, 65-70, 60-75, 70-80, or 75-80 IU/kg as a third administration. In some embodiments, rVWF is administered to the subject at a range of 40-80 IU/kg, e.g., 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 71, 72, 73, 74, 75, 76, 77, 78, 79, 80, 40-80, 45-80, 50-80, 45-70, 45-60, 45-55, 45-50, 50-60, 55-60, 60-65, 55-65, 60-70, 65-70, 60-75, 70-80, or 75-80 IU/kg as a subsequent administration.

Compositions of rVWF can be contained in pharmaceutical formulations, as described herein. Such formulations can be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

In one aspect, formulations of the invention are administered by an initial bolus followed by a continuous infusion to maintain therapeutic circulating levels of drug product. As another example, the inventive compound is administered as a one-time dose. Those of ordinary skill in the art will readily optimize effective dosages and administration regimens as determined by good medical practice and the clinical condition of the individual patient. The route of administration can be, but is not limited to, by intravenous, intraperitoneal, subcutaneous, or intramuscular administration. The frequency of dosing depends on the pharmacokinetic parameters of the agents and the route of administration. The optimal pharmaceutical formulation is determined by one skilled in the art depending upon the route of administration and desired dosage. See for example, Remington's Pharmaceutical Sciences, 18th Ed., 1990, Mack Publishing Co., Easton, Pa. 18042 pages 1435-1712, the disclosure of which is hereby incorporated by reference in its entirety for all purposes and in particular for all teachings related to formulations, routes of administration and dosages for pharmaceutical products. Such formulations influence the physical state, stability, rate of in vivo release, and rate of in vivo clearance of the administered agents. Depending on the route of administration, a suitable dose is calculated according to body weight, body surface area or organ size. Appropriate dosages may be ascertained through use of established assays for determining blood level dosages in conjunction with appropriate dose-response data. The final dosage regimen is determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. By way of example, a typical dose of a recombinant VWF of the present invention is approximately 50 IU/kg, equal to 500 μg/kg. As studies are conducted, further information will emerge regarding the appropriate dosage levels and duration of treatment for various diseases and conditions.

To administer compositions to human or test animals, in one aspect, the compositions comprises one or more pharmaceutically acceptable carriers. The phrases “pharmaceutically” or “pharmacologically” acceptable refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

The pharmaceutical formulations are administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, and/or intrapulmonary injection at a particular site is contemplated as well. Generally, compositions are essentially free of pyrogens, as well as other impurities that could be harmful to the recipient.

Single or multiple administrations of rVWF are carried out with the dose levels and pattern being selected by the treating physician. For the prevention or treatment of disease, the appropriate dosage depends on the type of disease to be treated (e.g., von Willebrand disease), the severity and course of the disease, whether drug is administered for preventive or therapeutic purposes, previous therapy, the patient's clinical history and response to the drug, and the discretion of the attending physician.

a. Lyophilized VWF Formulations

The present method also provides formulations of rVWF for use in the treatment methods provided herein. In some embodiments, the rVWF composition is used for the production of a pharmaceutical composition. In some embodiments, the rVWF can be formulated into a lyophilized formulation.

In some embodiments, the formulations comprising a VWF polypeptide of the invention are lyophilized after purification and prior to administration to a subject. Lyophilization is carried out using techniques common in the art and should be optimized for the composition being developed (Tang et al., Pharm Res. 21:191-200, (2004) and Chang et al., Pharm Res. 13:243-9 (1996)).

A lyophilization cycle is, in one aspect, composed of three steps: freezing, primary drying, and secondary drying (A. P. Mackenzie, Phil Trans R Soc London, Ser B, Biol 278:167 (1977)). In the freezing step, the solution is cooled to initiate ice formation. Furthermore, this step induces the crystallization of the bulking agent. The ice sublimes in the primary drying stage, which is conducted by reducing chamber pressure below the vapor pressure of the ice, using a vacuum and introducing heat to promote sublimation. Finally, adsorbed or bound water is removed at the secondary drying stage under reduced chamber pressure and at an elevated shelf temperature. The process produces a material known as a lyophilized cake. Thereafter the cake can be reconstituted with either sterile water or suitable diluent for injection.

The lyophilization cycle not only determines the final physical state of excipients but also affects other parameters such as reconstitution time, appearance, stability and final moisture content. The composition structure in the frozen state proceeds through several transitions (e.g., glass transitions, wettings, and crystallizations) that occur at specific temperatures and the structure may be used to understand and optimize the lyophilization process. The glass transition temperature (Tg and/or Tg′) can provide information about the physical state of a solute and can be determined by differential scanning calorimetry (DSC). Tg and Tg′ are an important parameter that must be taken into account when designing the lyophilization cycle. For example, Tg′ is important for primary drying. Furthermore, in the dried state, the glass transition temperature provides information on the storage temperature of the final product.

b. Pharmaceutical Formulations and Excipients in General

Excipients are additives that either impart or enhance the stability and delivery of a drug product (e.g., protein). Regardless of the reason for their inclusion, excipients are an integral component of a formulation and therefore need to be safe and well tolerated by patients. For protein drugs, the choice of excipients is particularly important because they can affect both efficacy and immunogenicity of the drug. Hence, protein formulations need to be developed with appropriate selection of excipients that afford suitable stability, safety, and marketability.

A lyophilized formulation is, in one aspect, at least comprised of one or more of a buffer, a bulking agent, and a stabilizer. In this aspect, the utility of a surfactant is evaluated and selected in cases where aggregation during the lyophilization step or during reconstitution becomes an issue. An appropriate buffering agent is included to maintain the formulation within stable zones of pH during lyophilization. A comparison of the excipient components contemplated for liquid and lyophilized protein formulations is provided in Table 10.

TABLE 10
Excipient components of lyophilized protein formulations

| Excipient | |
|---|---|
| component | Function in lyophilized formulation |
| Buffer | Maintain pH of formulation during |
| | lyophilization and upon reconstitution |
| Tonicity agent/ | Stabilizers include cryo and lycoprotectants |
| stabilizer | Examples include Polyols, sugars and |
| | polymers |
| | Cryoprotectants protect proteins from |
| | freezing stresses |
| | Lyoprotectants stabilize proteins in the freeze- |
| | dried state |
| Bulking agent | Used to enhance product elegance and to |
| | prevent blowout |
| | Provides structural strength to the lyo cake |
| | Examples include mannitol and glycine |
| Surfactant | Employed if aggregation during the |
| | lyophilization process is an issue |
| | May serve to reduce reconstitution times |
| | Examples include polysorbate 20 and 80 |
| Anti-oxidant | Usually not employed, molecular reactions in |
| | the lyo cake are generally retarded |
| Metal ions/ | May be included if a specific metal ion is |
| chelating agent | included only as a co-factor of where the |
| | metal is required for protease activity |
| | Chelating agents are generally not needed in |
| | lyo formulations |
| Preservative | For multi-dose formulations only |
| | Provides protection against microbial growth |
| | in formulation |
| | Is usually included in the reconstitution |
| | diluent (e.g., bWFI) |

The principal challenge in developing formulations for proteins is stabilizing the product against the stresses of manufacturing, shipping and storage. The role of formulation excipients is to provide stabilization against these stresses. Excipients are also employed to reduce viscosity of high concentration protein formulations in order to enable their delivery and enhance patient convenience. In general, excipients can be classified on the basis of the mechanisms by which they stabilize proteins against various chemical and physical stresses. Some excipients are used to alleviate the effects of a specific stress or to regulate a particular susceptibility of a specific protein. Other excipients have more general effects on the physical and covalent stabilities of proteins. The excipients described herein are organized either by their chemical type or their functional role in formulations. Brief descriptions of the modes of stabilization are provided when discussing each excipient type.

Given the teachings and guidance provided herein, those skilled in the art will know what amount or range of excipient can be included in any particular formulation to achieve a biopharmaceutical formulation of the invention that promotes retention in stability of the biopharmaceutical (e.g., a protein). For example, the amount and type of a salt to be included in a biopharmaceutical formulation of the invention is selected based on the desired osmolality (e.g., isotonic, hypotonic or hypertonic) of the final solution as well as the amounts and osmolality of other components to be included in the formulation.

By way of example, inclusion of about 5% sorbitol can achieve isotonicity while about 9% of a sucrose excipient is needed to achieve isotonicity. Selection of the amount or range of concentrations of one or more excipients that can be included within a biopharmaceutical formulation of the invention has been exemplified above by reference to salts, polyols and sugars. However, those skilled in the art will understand that the considerations described herein and further exemplified by reference to specific excipients are equally applicable to all types and combinations of excipients including, for example, salts, amino acids, other tonicity agents, surfactants, stabilizers, bulking agents, cryoprotectants, lyoprotectants, anti-oxidants, metal ions, chelating agents and/or preservatives.

Further, where a particular excipient is reported in molar concentration, those skilled in the art will recognize that the equivalent percent (%) w/v (e.g., (grams of substance in a solution sample/mL of solution)×100%) of solution is also contemplated.

Of course, a person having ordinary skill in the art would recognize that the concentrations of the excipients described herein share an interdependency within a particular formulation. By way of example, the concentration of a bulking agent may be lowered where, e.g., there is a high protein concentration or where, e.g., there is a high stabilizing agent concentration. In addition, a person having ordinary skill in the art would recognize that, in order to maintain the isotonicity of a particular formulation in which there is no bulking agent, the concentration of a stabilizing agent would be adjusted accordingly (e.g., a “tonicifying” amount of stabilizer would be used). Common excipients are known in the art and can be found in Powell et al., Compendium of Excipients for Parenteral Formulations (1998), PDA J. Pharm. Sci. Technology, 52:238-311.

c. Pharmaceutical Buffers and Buffering Agents

The stability of a pharmacologically active protein formulation is usually observed to be maximal in a narrow pH range. This pH range of optimal stability needs to be identified early during pre-formulation studies. Several approaches, such as accelerated stability studies and calorimetric screening studies, are useful in this endeavor (Remmele R. L. Jr., et al., Biochemistry, 38(16): 5241-7 (1999)). Once a formulation is finalized, the protein must be manufactured and maintained throughout its shelf-life. Hence, buffering agents are almost always employed to control pH in the formulation.

The buffer capacity of the buffering species is maximal at a pH equal to the pKa and decreases as pH increases or decreases away from this value. Ninety percent of the buffering capacity exists within one pH unit of its pKa. Buffer capacity also increases proportionally with increasing buffer concentration.

Several factors need to be considered when choosing a buffer. First and foremost, the buffer species and its concentration need to be defined based on its pKa and the desired formulation pH. Equally important is to ensure that the buffer is compatible with the protein and other formulation excipients, and does not catalyze any degradation reactions. A third important aspect to be considered is the sensation of stinging and irritation the buffer may induce upon administration. For example, citrate is known to cause stinging upon injection (Laursen T, et al., Basic Clin Pharmacol Toxicol., 98(2): 218-21 (2006)). The potential for stinging and irritation is greater for drugs that are administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored. One has to be particularly careful about potassium ions administered in the form of the potassium phosphate buffer, which can induce cardiovascular effects in a patient (Hollander-Rodriguez J C, et al., Am. Fam. Physician., 73(2): 283-90 (2006)).

Buffers for lyophilized formulations need additional consideration. Some buffers like sodium phosphate can crystallize out of the protein amorphous phase during freezing resulting in shifts in pH. Other common buffers such as acetate and imidazole may sublime or evaporate during the lyophilization process, thereby shifting the pH of formulation during lyophilization or after reconstitution.

The buffer system present in the compositions is selected to be physiologically compatible and to maintain a desired pH of the pharmaceutical formulation. In one embodiment, the pH of the solution is between pH 2.0 and pH 12.0. For example, the pH of the solution may be 2.0, 2.3, 2.5, 2.7, 3.0, 3.3, 3.5, 3.7, 4.0, 4.3, 4.5, 4.7, 5.0, 5.3, 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, 10.0, 10.3, 10.5, 10.7, 11.0, 11.3, 11.5, 11.7, or 12.0.

The pH buffering compound may be present in any amount suitable to maintain the pH of the formulation at a predetermined level. In one embodiment, the pH buffering concentration is between 0.1 mM and 500 mM (1 M). For example, it is contemplated that the pH buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM.

Exemplary pH buffering agents used to buffer the formulation as set out herein include, but are not limited to organic acids, glycine, histidine, glutamate, succinate, phosphate, acetate, citrate, Tris, HEPES, and amino acids or mixtures of amino acids, including, but not limited to aspartate, histidine, and glycine. In one embodiment of the present invention, the buffering agent is citrate.

d. Pharmaceutical Stabilizers and Bulking Agents

In one aspect of the present pharmaceutical formulations, a stabilizer (or a combination of stabilizers) is added to prevent or reduce storage-induced aggregation and chemical degradation. A hazy or turbid solution upon reconstitution indicates that the protein has precipitated or at least aggregated. The term “stabilizer” means an excipient capable of preventing aggregation or physical degradation, including chemical degradation (for example, autolysis, deamidation, oxidation, etc.) in an aqueous state. Stabilizers contemplated include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methyl pyrollidene, cellulose and hyaluronic acid, sodium chloride, (Carpenter et al., Develop. Biol. Standard 74:225, (1991)). In the present formulations, the stabilizer is incorporated in a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. In one embodiment of the present invention, mannitol and trehalose are used as stabilizing agents.

If desired, the formulations also include appropriate amounts of bulking and osmolality regulating agents. Bulking agents include, for example and without limitation, mannitol, glycine, sucrose, polymers such as dextran, polyvinylpyrolidone, carboxymethylcellulose, lactose, sorbitol, trehalose, or xylitol. In one embodiment, the bulking agent is mannitol. The bulking agent is incorporated in a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM.

e. Pharmaceutical Surfactants

Proteins have a high propensity to interact with surfaces making them susceptible to adsorption and denaturation at air-liquid, vial-liquid, and liquid-liquid (silicone oil) interfaces. This degradation pathway has been observed to be inversely dependent on protein concentration and results in either the formation of soluble and insoluble protein aggregates or the loss of protein from solution via adsorption to surfaces. In addition to container surface adsorption, surface-induced degradation is exacerbated with physical agitation, as would be experienced during shipping and handling of the product.

Surfactants are commonly used in protein formulations to prevent surface-induced degradation. Surfactants are amphipathic molecules with the capability of out-competing proteins for interfacial positions. Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serves to prevent protein molecules from adsorbing at the interface. Thereby, surface-induced degradation is minimized. Surfactants contemplated herein include, without limitation, fatty acid esters of sorbitan polyethoxylates, e.g., polysorbate 20 and polysorbate 80. The two differ only in the length of the aliphatic chain that imparts hydrophobic character to the molecules, C-12 and C-18, respectively. Accordingly, polysorbate-80 is more surface-active and has a lower critical micellar concentration than polysorbate-20.

Detergents can also affect the thermodynamic conformational stability of proteins. Here again, the effects of a given detergent excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (e.g. increasing the exposure of hydrophobic portions of the protein molecule in complement to binding polysorbate). Alternatively, if the protein native state exhibits some hydrophobic surfaces, detergent binding to the native state may stabilize that conformation.

Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon during freezing and drying (Chang, B, J. Pharm. Sci. 85:1325, (1996)). Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfo succinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, 60, 65 and 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. Compositions comprising these surfactants, either individually or as a mixture in different ratios, are therefore further provided. In one embodiment of the present invention, the surfactant is TWEEN-80. In the present formulations, the surfactant is incorporated in a concentration of about 0.01 to about 0.5 g/L. In formulations provided, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 g/L.

f. Pharmaceutical Salts

Salts are often added to increase the ionic strength of the formulation, which can be important for protein solubility, physical stability, and isotonicity. Salts can affect the physical stability of proteins in a variety of ways. Ions can stabilize the native state of proteins by binding to charged residues on the protein's surface. Alternatively, salts can stabilize the denatured state by binding to peptide groups along the protein backbone (—CONH—). Salts can also stabilize the protein native conformation by shielding repulsive electrostatic interactions between residues within a protein molecule. Salts in protein formulations can also shield attractive electrostatic interactions between protein molecules that can lead to protein aggregation and insolubility. In formulations provided, the salt concentration is between 0.1, 1, 10, 20, 30, 40, 50, 80, 100, 120, 150, 200, 300, and 500 mM.

g. Other Common Excipient Components: Pharmaceutical Amino Acids

Amino acids have found versatile use in protein formulations as buffers, bulking agents, stabilizers and antioxidants. Thus, in one aspect histidine and glutamic acid are employed to buffer protein formulations in the pH range of 5.5-6.5 and 4.0-5.5 respectively. The imidazole group of histidine has a pKa=6.0 and the carboxyl group of glutamic acid side chain has a pKa of 4.3 which makes these amino acids suitable for buffering in their respective pH ranges. Glutamic acid is particularly useful in such cases. Histidine is commonly found in marketed protein formulations, and this amino acid provides an alternative to citrate, a buffer known to sting upon injection. Interestingly, histidine has also been reported to have a stabilizing effect, with respect to aggregation when used at high concentrations in both liquid and lyophilized presentations (Chen B, et al., Pharm Res., 20(12): 1952-60 (2003)). Histidine was also observed by others to reduce the viscosity of a high protein concentration formulation. However, in the same study, the authors observed increased aggregation and discoloration in histidine containing formulations during freeze-thaw studies of the antibody in stainless steel containers. Another note of caution with histidine is that it undergoes photo-oxidation in the presence of metal ions (Tomita M, et al., Biochemistry, 8(12): 5149-60 (1969)). The use of methionine as an antioxidant in formulations appears promising; it has been observed to be effective against a number of oxidative stresses (Lam X M, et al., J Pharm ScL, 86(11): 1250-5 (1997)).

In various aspects, formulations are provided which include one or more of the amino acids glycine, proline, serine, arginine and alanine have been shown to stabilize proteins by the mechanism of preferential exclusion. Glycine is also a commonly used bulking agent in lyophilized formulations. Arginine has been shown to be an effective agent in inhibiting aggregation and has been used in both liquid and lyophilized formulations.

In formulations provided, the amino acid concentration is between 0.1, 1, 10, 20, 30, 40, 50, 80, 100, 120, 150, 200, 300, and 500 mM. In one embodiment of the present invention, the amino acid is glycine.

h. Other Common Excipient Components: Pharmaceutical Antioxidants

Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The invention therefore contemplates the use of the pharmaceutical antioxidants including, without limitation, reducing agents, oxygen/free-radical scavengers, or chelating agents. Antioxidants in therapeutic protein formulations are, in one aspect, water-soluble and remain active throughout the product shelf-life. Reducing agents and oxygen/free-radical scavengers work by ablating active oxygen species in solution. Chelating agents such as EDTA are effective by binding trace metal contaminants that promote free-radical formation. For example, EDTA was utilized in the liquid formulation of acidic fibroblast growth factor to inhibit the metal ion catalyzed oxidation of cysteine residues.

In addition to the effectiveness of various excipients to prevent protein oxidation, the potential for the antioxidants themselves to induce other covalent or physical changes to the protein is of concern. For example, reducing agents can cause disruption of intramolecular disulfide linkages, which can lead to disulfide shuffling. In the presence of transition metal ions, ascorbic acid and EDTA have been shown to promote methionine oxidation in a number of proteins and peptides (Akers M J, and Defelippis M R. Peptides and Proteins as Parenteral Solutions. In: Pharmaceutical Formulation Development of Peptides and Proteins. Sven Frokjaer, Lars Hovgaard, editors. Pharmaceutical Science. Taylor and Francis, UK (1999)); Fransson J. R., /. Pharm. Sci. 86(9): 4046-1050 (1997); Yin J, et al., Pharm Res., 21(12): 2377-83 (2004)). Sodium thiosulfate has been reported to reduce the levels of light and temperature induced methionine-oxidation in rhuMab HER2; however, the formation of a thiosulfate-protein adduct was also reported in this study (Lam X M, Yang J Y, et al., J Pharm Sci. 86(11): 1250-5 (1997)). Selection of an appropriate antioxidant is made according to the specific stresses and sensitivities of the protein. Antioxidants contemplated in certain aspects include, without limitation, reducing agents and oxygen/free-radical scavengers, EDTA, and sodium thiosulfate.

i. Other Common Excipient Components: Pharmaceutical Metal Ions

In general, transition metal ions are undesired in protein formulations because they can catalyze physical and chemical degradation reactions in proteins. However, specific metal ions are included in formulations when they are co-factors to proteins and in suspension formulations of proteins where they form coordination complexes (e.g., zinc suspension of insulin). Recently, the use of magnesium ions (10-120 mM) has been proposed to inhibit the isomerization of aspartic acid to isoaspartic acid (WO 2004039337).

Two examples where metal ions confer stability or increased activity in proteins are human deoxyribonuclease (rhDNase, Pulmozyme®), and Factor VIII. In the case of rhDNase, Ca′ ions (up to 100 mM) increased the stability of the enzyme through a specific binding site (Chen B, et al., Pharm Sci., 88(4): 477-82 (1999)). In fact, removal of calcium ions from the solution with EGTA caused an increase in deamidation and aggregation. However, this effect was observed only with Ca⁺² ions; other divalent cations Mg⁺², Mn⁺² and Zn⁺² were observed to destabilize rhDNase. Similar effects were observed in Factor VIII. Ca⁺² and Sr⁺² ions stabilized the protein while others like Mg⁺², Mn⁺² and Zn⁺², Cu⁺² and Fe⁺² destabilized the enzyme (Fatouros, A., et al., Int. J. Pharm., 155, 121-131 (1997). In a separate study with Factor VIII, a significant increase in aggregation rate was observed in the presence of Al⁺³ ions (Derrick T S, et al., I. Pharm. Sci., 93(10): 2549-57 (2004)). The authors note that other excipients like buffer salts are often contaminated with Al⁺³ ions and illustrate the need to use excipients of appropriate quality in formulated products.

j. Other Common Excipient Components: Pharmaceutical Preservatives

Preservatives are necessary when developing multi-use parenteral formulations that involve more than one extraction from the same container. Their primary function is to inhibit microbial growth and ensure product sterility throughout the shelf-life or term of use of the drug product. Commonly used preservatives include, without limitation, benzyl alcohol, phenol and m-cresol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy S, et al., J Pharm ScL, 94(2): 382-96 (2005)).

To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin® (liquid, Novo Nordisk), Nutropin AQ® (liquid, Genentech) & Genotropin (lyophilized—dual chamber cartridge, Pharmacia & Upjohn) contain phenol while Somatrope® (Eli Lilly) is formulated with m-cresol.

Several aspects need to be considered during the formulation development of preserved dosage forms. The effective preservative concentration in the drug product must be optimized. This requires testing a given preservative in the dosage form with concentration ranges that confer anti-microbial effectiveness without compromising protein stability. For example, three preservatives were successfully screened in the development of a liquid formulation for interleukin-1 receptor (Type I), using differential scanning calorimetry (DSC). The preservatives were rank ordered based on their impact on stability at concentrations commonly used in marketed products (Remmele R L Jr., et al., Pharm Res., 15(2): 200-8 (1998)).

Development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein significantly minimizing the associated stability risks. With liquid formulations, preservative effectiveness and stability have to be maintained over the entire product shelf-life (−18-24 months). An important point to note is that preservative effectiveness has to be demonstrated in the final formulation containing the active drug and all excipient components.

Some preservatives can cause injection site reactions, which is another factor that needs consideration when choosing a preservative. In clinical trials that focused on the evaluation of preservatives and buffers in Norditropin, pain perception was observed to be lower in formulations containing phenol and benzyl alcohol as compared to a formulation containing m-cresol (Kappelgaard A. M., Horm Res. 62 Suppl 3:98-103 (2004)). Interestingly, among the commonly used preservative, benzyl alcohol possesses anesthetic properties (Minogue S C, and Sun DA., AnesthAnalg., 100(3): 683-6 (2005)). In various aspects the use of preservatives provides a benefit that outweighs any side effects.

k. Methods of Preparation of Pharmaceutical Formulations

The present invention further contemplates methods for the preparation of pharmaceutical formulations.

The present methods further comprise one or more of the following steps: adding a stabilizing agent as described herein to said mixture prior to lyophilizing, adding at least one agent selected from a bulking agent, an osmolality regulating agent, and a surfactant, each of which as described herein, to said mixture prior to lyophilization.

The standard reconstitution practice for lyophilized material is to add back a volume of pure water or sterile water for injection (WFI) (typically equivalent to the volume removed during lyophilization), although dilute solutions of antibacterial agents are sometimes used in the production of pharmaceuticals for parenteral administration (Chen, Drug Development and Industrial Pharmacy, 18:1311-1354 (1992)). Accordingly, methods are provided for preparation of reconstituted rVWF compositions comprising the step of adding a diluent to a lyophilized rVWF composition of the invention.

The lyophilized material may be reconstituted as an aqueous solution. A variety of aqueous carriers, e.g., sterile water for injection, water with preservatives for multi dose use, or water with appropriate amounts of surfactants (for example, an aqueous suspension that contains the active compound in admixture with excipients suitable for the manufacture of aqueous suspensions). In various aspects, such excipients are suspending agents, for example and without limitation, sodium carboxymethylcellulose, methylcellulose, hydroxypropylmethylcellulose, sodium alginate, polyvinylpyrrolidone, gum tragacanth and gum acacia; dispersing or wetting agents are a naturally-occurring phosphatide, for example and without limitation, lecithin, or condensation products of an alkylene oxide with fatty acids, for example and without limitation, polyoxyethylene stearate, or condensation products of ethylene oxide with long chain aliphatic alcohols, for example and without limitation, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example and without limitation, polyethylene sorbitan monooleate. In various aspects, the aqueous suspensions also contain one or more preservatives, for example and without limitation, ethyl, or n-propyl, p-hydroxybenzoate.

l. Exemplary rVWF Formulation for Administration

In some embodiments, the present methods provide for an enhanced formulation that allows a final product with high potency (high rVWF concentration and enhanced long term stability) in order to reduce the volume for the treatment (100 IU/ml to 10000 IU/ml). In some embodiments, the rVWF concentration in the formulation for administration is about 100 IU/ml to 10000 IU/ml. In some embodiments, the rVWF concentration in the formulation for administration is about 500 IU/ml to 10000 IU/ml. In some embodiments, the rVWF concentration in the formulation for administration is about 1000 IU/ml to 10000 IU/ml. In some embodiments, the rVWF concentration in the formulation for administration is about 2000 IU/ml to 10000 IU/ml. In some embodiments, the rVWF concentration in the formulation for administration is about 3000 IU/ml to 10000 IU/ml. In some embodiments, the rVWF concentration in the formulation for administration is about 4000 IU/ml to 10000 IU/ml. In some embodiments, the rVWF concentration in the formulation for administration is about 5000 IU/ml to 10000 IU/ml. In some embodiments, the rVWF concentration in the formulation for administration is about 6000 IU/ml to 10000 IU/ml. In some embodiments, the rVWF concentration in the formulation for administration is about 7000 IU/ml to 10000 IU/ml. In some embodiments, the rVWF concentration in the formulation for administration is about 8000 IU/ml to 10000 IU/ml. In some embodiments, the rVWF concentration in the formulation for administration is about 9000 IU/ml to 10000 IU/ml.

In some embodiments, the formulation for administration comprises one or more zwitterionic compounds, including for example, amino acids like Histidine, Glycine, Arginine. In some embodiments, the formulation for administration comprises a component with amphipathic characteristic having a minimum of one hydrophobic and one hydrophilic group, including for example polysorbate 80, octylpyranosid, dipeptides, and/or amphipathic peptides. In some embodiments, the formulation for administration comprises a non reducing sugar or sugar alcohol or disaccharides, including for example, sorbitol, mannitol, sucrose, or trehalose. In some embodiments, the formulation for administration comprises a nontoxic water soluble salt, including for example, sodium chloride, that results in a physiological osmolality. In some embodiments, the formulation for administration comprises a pH in a range from 6.0 to 8.0. In some embodiments, the formulation for administration comprises a pH of about 6.0, about 6.5, about 7, about 7.5 or about 8.0. In some embodiments, the formulation for administration comprises one or more bivalent cations that stabilize rVWF, including for example, Ca2+, Mg2+, Zn2+, Mn2+ and/or combinations thereof. In some embodiments, the formulation for administration comprises about 1 mM to about 50 mM Glycine, about 1 mM to about 50 mM Histidine, about zero to about 300 mM sodium chloride (e.g., less than 300 mM sodium), about 0.01% to about 0.05% polysorbate 20 (or polysorbate 80), and about 0.5% to about 20% (w/w) sucrose with a pH of about 7.0 and having a physiological osmolarity at the time point of administration.

In some embodiments, the formulation for administration can be freeze dried. In some embodiments, the formulation for administration is stable and can be stored in liquid state at about 2° C. to about 8° C., as well as at about 18° C. to about 25° C. In some embodiments, the formulation for administration is stable and can be stored in liquid state at about 2° C. to about 8° C. In some embodiments, the formulation for administration is stable and can be stored in liquid state at about 18° C. to about 25° C.

V. Administration of rVWF for Methods of Prophylactic Treatment in Patients with Severe VWD

In some embodiments, the present invention provides for prophylactically treating spontaneous bleeding episodes in a subject with severe von Willebrand Disease (VWD). In some embodiments, the prophylactic treatment comprises administering to the subject a recombinant von Willebrand Factor (rVWF) in order to reduce the frequency and/or duration of spontaneous bleeding episodes.

In some embodiments, spontaneous bleeding episodes include any episode not related to trauma. In some embodiments, the efficacy of the treatment is indicated by a reduction in the number of spontaneous bleeding episodes. In some embodiments, a reduction is the number of spontaneous bleeding episodes is indicated by a reduction in the annual bleeding rate (ABR). In some embodiments, the pretreatment ABR is determined based on the following formula: number of bleeds/days not on treatment regimen. In some embodiments, the pretreatment ABR is determined based on the following formula: number of bleeds/12 months prior to the prophylactic treatment with rVWF. In some embodiments, the prophylactic ABR (ABR after prophylactic treatment) is determined based on the following formula: number of bleeds/days on treatment regimen.

In some embodiments, a reduction of ≥25% in annual bleeding rate (ABR) for spontaneous bleeding episodes during rVWF prophylaxis relative to the pretreatment ABR is indicative of prophylactic treatment efficacy. In some embodiments, a reduction of ≥30% in annual bleeding rate (ABR) for spontaneous bleeding episodes during rVWF prophylactic treatment relative to the pretreatment ABR is indicative of prophylactic treatment efficacy. In some embodiments, a reduction of ≥35% in annual bleeding rate (ABR) for spontaneous bleeding episodes during rVWF prophylactic treatment relative to the pretreatment ABR is indicative of prophylactic treatment efficacy. In some embodiments, a reduction of ≥40% in annual bleeding rate (ABR) for spontaneous bleeding episodes during rVWF prophylactic treatment relative to the pretreatment ABR is indicative of prophylactic treatment efficacy. In some embodiments, a reduction of ≥45% in annual bleeding rate (ABR) for spontaneous bleeding episodes during rVWF prophylactic treatment relative to the pretreatment ABR is indicative of prophylactic treatment efficacy. In some embodiments, a reduction of ≥50% in annual bleeding rate (ABR) for spontaneous bleeding episodes during rVWF prophylactic treatment relative to the pretreatment ABR is indicative of prophylactic treatment efficacy. In some embodiments, a reduction of ≥55% in annual bleeding rate (ABR) for spontaneous bleeding episodes during rVWF prophylactic treatment relative to the pretreatment ABR is indicative of prophylactic treatment efficacy. In some embodiments, a reduction of ≥60% in annual bleeding rate (ABR) for spontaneous bleeding episodes during rVWF prophylactic treatment relative to the pretreatment ABR is indicative of prophylactic treatment efficacy. In some embodiments, a reduction of ≥65% in annual bleeding rate (ABR) for spontaneous bleeding episodes during rVWF prophylactic treatment relative to the pretreatment ABR is indicative of prophylactic treatment efficacy. In some embodiments, a reduction of ≥70% in annual bleeding rate (ABR) for spontaneous bleeding episodes during rVWF prophylactic treatment relative to the pretreatment ABR is indicative of prophylactic treatment efficacy. In some embodiments, a reduction of ≥75% in annual bleeding rate (ABR) for spontaneous bleeding episodes during rVWF prophylactic treatment relative to the pretreatment ABR is indicative of prophylactic treatment efficacy. In some embodiments, a reduction of ≥80% in annual bleeding rate (ABR) for spontaneous bleeding episodes during rVWF prophylactic treatment relative to the pretreatment ABR is indicative of prophylactic treatment efficacy. In some embodiments, a reduction of ≥85% in annual bleeding rate (ABR) for spontaneous bleeding episodes during rVWF prophylactic treatment relative to the pretreatment ABR is indicative of prophylactic treatment efficacy. In some embodiments, a reduction of ≥90% in annual bleeding rate (ABR) for spontaneous bleeding episodes during rVWF prophylactic treatment relative to the pretreatment ABR is indicative of prophylactic treatment efficacy.

In some embodiments, prophylactic treatment efficacy can be measured by obtaining samples and examining vWF:RCo and/or FVIII activities before and after prophylactic treatment with rVWF. In some embodiments, samples for examining FVIII, FVIII:C, VWF:RCo, VWF:Ag, and/or VWF collagen-binding capacity activities are obtained prior to the prophylactic treatment with rVWF and after the prophylactic treatment with rVWF. In some embodiments, samples for examining FVIII, FVIII:C, VWF:RCo, VWF:Ag, and VWF collagen-binding capacity activity are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF. In some embodiments, samples for examining FVIII, FVIII:C, VWF:RCo, VWF:Ag, and VWF collagen-binding capacity activity are obtained 25 to 31 days after prophylactic treatment with rVWF. In some embodiments, samples for FVIII, FVIII:C, VWF:RCo, VWF:Ag, and VWF collagen-binding capacity activity are obtained after or during a bleeding episode and in such embodiments, a sample is take prior to rVWF administration, 2 hours after administration and then every 12-24 hours until resolution of the bleeding event. In some embodiments, treatment efficacy can be determined after or during a bleeding episode, and in such embodiments, samples for examining FVIII, FVIII:C, VWF:RCo, VWF:Ag, and/or VWF collagen-binding capacity activities are obtained after the bleeding episode, and further wherein the samples are obtained prior to rVWF administration, 2 hours after administration and then every 12-24 hours until resolution of the bleeding event. In some embodiments, FVIII, FVIII:C, VWF:RCo, VWF:Ag, and VWF collagen-binding capacity activity versus time profiles are determined based on the samples in order to monitor treatment efficacy for the prophylactic treatment with rVWF. In some embodiments, FVIII:C is also measured by a 1-stage clotting assay versus time profiles in order to monitor treatment efficacy for the prophylactic treatment with rVWF. In some embodiments, FVIII, FVIII:C, VWF:RCo, VWF:Ag, and/or VWF collagen-binding capacity activity levels are improved after the prophylactic treatment with rVWF as compared to the levels prior to the prophylactic treatment with rVWF. Ira some embodiments, FVIII, FVIII:C, VWF/RCo, VWF:Ag, and/or VWF collagen-binding capacity activity levels are improved after the prophylactic treatment with rVWF as compared to the levels prior to the prophylactic treatment with rVWF and this improvement is indicative of treatment efficacy. In some embodiments, prophylactic treatment efficacy is indicated by an improvement in FVIII, FVIII:C, VWF:RCo, VWF:Ag, and/or VWF collagen-binding capacity activity levels after the prophylactic treatment with rVWF as compared to the levels prior to the prophylactic treatment with rVWF. In some embodiments, an improvement in FVIII, FVIII:C, VWF:RCo, VWF:Ag, and/or VWF collagen-binding capacity activity levels includes a change in the activity levels such that the activity levels are closer to normal levels, e.g., levels in a subject that does not have VWD.

In some aspects, prophylactic treatment efficacy of rVWF administration is determined after or during a bleeding episode. In some embodiments, samples for examining FVIII, FVIII:C, VWF:RCo, VWF:Ag, and/or VWF collagen-binding capacity activities are obtained after the bleeding episode. In some embodiments, samples for examining FVIII, FVIII:C, VWF:RCo, VWF:Ag, and/or VWF collagen-binding capacity activities are obtained during the bleeding episode. In some instances, samples are obtained from a patient during a bleeding episode such that the FVIII, FVIII:C, VWF:RCo, VWF:Ag, and/or VWF collagen-binding capacity activities can be determined using the samples. In other instances, samples are obtained from a patient after a bleeding episode. In some embodiments the samples are obtained prior to rVWF administration. In some embodiments the samples are obtained after rVWF administration. In some embodiments the samples are obtained 2 hours after rVWF administration. In some embodiments the samples are obtained every 12-24 hours until resolution of the bleeding episode. In certain embodiments, samples are obtained prior to rVWF administration and 2 hours after rVWF administration. In some embodiments, samples are obtained prior to rVWF administration and every 12-24 hours until resolution of the bleeding episode. In other embodiments, samples are obtained prior to rVWF administration, 2 hours after rVWF administration and every 12-24 hours until resolution of the bleeding episode. As such, the samples can be obtained prior to rVWF administration and prior to a bleeding episode. In some embodiments, samples are obtained after rVWF administration and prior to a bleeding episode. In some embodiments, samples are obtained after rVWF administration and during a bleeding episode. In certain embodiments, samples are obtained after rVWF administration and after resolution of a bleeding episode.

In some aspects, rVWF is administered to a subject at a range from 40-80 IU/kg, e.g., 40, 45, 50, 55, 60, 65, 70, 75, 80, 90, 100, 40-100, 40-80, 50-80, 60-80, 70-80, 40-50, 40-60, 40-70, 40-50, 50-60, 60-70, or 70-80 IU/kg. In some embodiments, rVWF is administered at least once a week to prevent a spontaneous bleeding episode. In some instances, the subject is given a single administration of rVWF. In some instances, the subject is administered a single infusion of rVWF.

In some aspects, rVWF is administered to a subject at a range from 40-80 IU/kg, e.g., 40, 45, 50, 55, 60, 65, 70, 75, 80, 90, 100, 40-100, 40-80, 50-80, 60-80, 70-80, 40-50, 40-60, 40-70, 40-50, 50-60, 60-70, or 70-80 IU/kg. In some embodiments, the dose ranges from 40 IU/kg to 60 IU/kg. In some embodiments, the dose ranges from 45 IU/kg to 55 IU/kg. In some embodiments, the dose is about 40 IU/kg, about 50 IU/kg, or about 60 IU/kg. In some embodiments, rVWF is administered at least two times each week to prevent a spontaneous bleeding episode. In other embodiments, rVWF is administered two or more times, e.g., 2, 3, 4, 5, or more times, a week to prevent a spontaneous bleeding episode. In some instances, the subject is given two administrations of rVWF. In some instances, the subject is administered two infusions of rVWF. In some embodiments, rVWF is administered twice a week. In some embodiments, rVWF is administered twice a week as an infusion dose ranging from 40 IU/kg to 60 IU/kg. In some embodiments, rVWF is administered twice a week as an infusion dose ranging from 40 IU/kg to 60 IU/kg by IV infusion. Each infusion can include a range from about 40-80 IU/kg rVWF, e.g., 40, 45, 50, 55, 60, 65, 70, 75, 80, 40-80, 50-80, 60-80, 70-80, 40-50, 40-60, 40-70, 40-50, 50-60, 60-70, or 70-80 IU/kg rVWF. In some embodiments, each infusion ranges from 40 IU/kg to 60 IU/kg. In some embodiments, each infusion ranges from 45 IU/kg to 55 IU/kg. In some embodiments, each infusion is about 40 IU/kg, about 50 IU/kg, or about 60 IU/kg. In some embodiments, the infusions can be substantially equal in amount. For instance, a first infusion and a second infusion can be substantially equal in amount. In some embodiments, the total dose of rVWF administered to the subject is about 40-160 IU/kg, e.g., 40-150, 40-125, 40-100, 40-90, 40-75, 50-150, 50-100, 75-150, 100-125, or 100-160 IU/kg.

In some aspects, rVWF is administered to a subject at a range from 40-80 IU/kg, e.g., 40, 45, 50, 55, 60, 65, 70, 75, 80, 90, 100, 40-100, 40-80, 50-80, 60-80, 70-80, 40-50, 40-60, 40-70, 40-50, 50-60, 60-70, or 70-80 IU/kg. In some embodiments, rVWF is administered at least two times each week to prevent a spontaneous bleeding episode. In some embodiments, rVWF is administered at least three times each week to prevent a spontaneous bleeding episode. In other embodiments, rVWF is administered three or more times, e.g., 3, 4, 5, or more times, a week to prevent a spontaneous bleeding episode. In some instances, the subject is given three administrations of rVWF. In some instances, the subject is administered three infusions of rVWF. Each infusion can include a range from about 40-80 IU/kg rVWF, e.g., 40, 45, 50, 55, 60, 65, 70, 75, 80, 40-80, 50-80, 60-80, 70-80, 40-50, 40-60, 40-70, 40-50, 50-60, 60-70, or 70-80 IU/kg rVWF. In some embodiments, the infusions can be substantially equal in amount. For instance, a first infusion, second infusion, and third infusion can be substantially equal in amount. In some embodiments, the total dose of rVWF administered to the subject is about 80-240 IU/kg, e.g., 120-240, 140-240, 140-200, 160-240, 180-240, 200-240, 80-120, 80-160, 80-200, 120-220, or 220-240 IU/kg. In some embodiments, the total dose of rVWF administered to the subject is less than about 160 IU/kg per week. In some embodiments, the total dose of rVWF administered to the subject is less than about 240 IU/kg per week.

In some embodiments, rVWF is administered at least once a week, at least twice (two times) a week, at least thrice (three times) a week, every day, every other day, every 2-3 days, every 2-4 days, every 2-5 days, and the like. In some instances, rVWF is administered for a total of 1 day, 2 days, 3 days, 4 days, 5 days, 6 days, or 7 days over the course of a 7-day period. In some embodiments, rVWF is administered on non-consecutive days. In some embodiments, rVWF is administered on consecutive days.

In some embodiments, rVWF is administered at least every 12 hours, 24 hours, 36 hour, 48 hours, 60 hours, 72 hours, 84 hours, or 96 hours. In some instances, rVWF is administered at least every 60 hours, 72 hours, or 84 hours. In some instances, rVWF is administered at least every 72 hours.

In some embodiments, recombinant Factor VIII (rFVIII) is also administered to the subject with severe VWD to prevent or reduce the frequency and/or duration of a spontaneous bleeding episode. In some cases, the treatment administered comprises rVWF and rFVIII. In other cases, the treatment administered does not include rFVIII. In some embodiments, rFVIII is administered to the subject at a range of about 10-70 IU/kg, e.g., 10-70, 10-60, 10-50, 10-40, 10-30, 10-20, 20-30, 30-40, 40-50, 50-60, or 60-70 IU/kg. In some instances, rFVIII is administered in the initial (first) dose or initial (first) infusion. In some instances, rFVIII is not administered in the initial (first) dose or initial (first) infusion. In some cases, rFVIII is administered as part of a second dose or second infusion. In some cases, rFVIII is not administered as part of a second dose or second infusion. In some cases, rFVIII is administered as part of a third dose or third infusion. In some cases, rFVIII is not administered as part of a third dose or third infusion.

In some embodiments, a subject with VWD who is at risk of having a spontaneous bleeding episode is administered a single infusion of rVWF and rFVIII. In some embodiments, the second administration of rVWF is not administered with FVIII. In some embodiments, the third administration of rVWF is not administered with FVIII.

In some embodiments, a subject with VWD who is at risk of having a spontaneous bleeding episode is administered a single infusion of rVWF and rFVIII. In some embodiments, the second administration of rVWF is administered with FVIII. In some embodiments, the third administration of rVWF is not administered with FVIII.

In some embodiments, a subject with VWD who is at risk of having a spontaneous bleeding episode is administered a single infusion of rVWF and rFVIII. In some embodiments, the second administration of rVWF is administered with FVIII. In some embodiments, the third administration of rVWF is administered with FVIII.

In some embodiments, a subject with VWD who is at risk of having a spontaneous bleeding episode is administered a single infusion of rVWF, and not rFVIII. In some embodiments, the second administration of rVWF is administered with FVIII. In some embodiments, the third administration of rVWF is administered with FVIII.

In some embodiments, a subject with VWD who is at risk of having a spontaneous bleeding episode is administered a single infusion of rVWF, and not rFVIII. In some embodiments, the second administration of rVWF is not administered with FVIII. In some embodiments, the third administration of rVWF is administered with FVIII.

In some embodiments, a subject with VWD who is at risk of having a spontaneous bleeding episode is administered a single infusion of rVWF, and not rFVIII. In some embodiments, the second administration of rVWF is administered with FVIII. In some embodiments, the third administration of rVWF is not administered with FVIII.

In some embodiments, a subject with VWD who is at risk of having a spontaneous bleeding episode is administered a first infusion and second infusion of rVWF. In some embodiments, the first and/or second infusion of rVWF is administered with FVIII.

In some embodiments, a subject with VWD who is at risk of having a spontaneous bleeding episode is administered a first infusion, second infusion, and third infusion of rVWF. In some embodiments, the first, second and/or third infusion of rVWF is administered with FVIII.

In some embodiments, of the method, when rVWF and FVIII are administered together, the rVWF to FVIII ratio is about 1.5:0.8. In some embodiments, of the method, when rVWF and FVIII are administered together, the rVWF to FVIII ratio is about 1.3:1. In some embodiments, of the method, when rVWF and FVIII are administered together, the rVWF to FVIII ratio is about 1.1:0.8. In some embodiments, of the method, when rVWF and FVIII are administered together, the rVWF to FVIII ratio is about 1.5:1. In some embodiments, of the method, when rVWF and FVIII are administered together, the rVWF to FVIII ratio is about 1.1:1.2.

In some embodiments, about 40 IU/kg rVWF of the rVWF is administered for prophylaxis of spontaneous bleeding. In some embodiments, about 45 IU/kg of the rVWF is administered for prophylaxis of spontaneous bleeding. In some embodiments, about 50 IU/kg of the rVWF is administered for prophylaxis of spontaneous bleeding. In some embodiments, about 55 IU/kg of the rVWF is administered for prophylaxis of spontaneous bleeding. In some embodiments, about 60 IU/kg of the rVWF is administered for prophylaxis of spontaneous bleeding. In some embodiments, about 65 IU/kg of the rVWF is administered for prophylaxis of spontaneous bleeding. In some embodiments, about 70 IU/kg of the rVWF is administered for prophylaxis of spontaneous bleeding. In some embodiments, about 75 IU/kg of the rVWF is administered for prophylaxis of spontaneous bleeding. In some embodiments, about 80 IU/kg of the rVWF is administered for prophylaxis of spontaneous bleeding. In some embodiments, about 40 IU/kg-80 IU/kg of the rVWF is administered for prophylaxis of spontaneous bleeding. In some embodiments, about 50 IU/kg-80 IU/kg of the rVWF is administered for prophylaxis of spontaneous bleeding. In some embodiments, about 40 IU/kg-70 IU/kg of the rVWF is administered for prophylaxis of spontaneous bleeding. In some embodiments, about 50 IU/kg-80 IU/kg of the rVWF is administered for prophylaxis of spontaneous bleeding. In some embodiments, about 40 IU/kg-60 IU/kg of the rVWF is administered for prophylaxis of spontaneous bleeding. In some embodiments, about 50 IU/kg-80 IU/kg of the rVWF is administered for prophylaxis of spontaneous bleeding.

In some embodiments, 40-80 IU/kg rVWF of the rVWF is administered twice per week for prophylactic treatment of spontaneous bleeding. In some embodiments, 50-80 IU/kg of the rVWF is administered twice per week for prophylactic treatment of spontaneous bleeding. In some embodiments, 40 IU/kg rVWF of the rVWF is administered twice per week for prophylactic treatment of spontaneous bleeding. In some embodiments, 45 IU/kg of the rVWF is administered twice per week for prophylactic treatment of spontaneous bleeding. In some embodiments, 50 IU/kg of the rVWF is administered twice per week for prophylactic treatment of spontaneous bleeding. In some embodiments, 55 IU/kg of the rVWF is administered twice per week for prophylactic treatment of spontaneous bleeding. In some embodiments, 60 IU/kg of the rVWF is administered twice per week for prophylactic treatment of spontaneous bleeding. In some embodiments, 65 IU/kg of the rVWF is administered twice per week for prophylactic treatment of spontaneous bleeding. In some embodiments, 70 IU/kg of the rVWF is administered twice per week for prophylactic treatment of spontaneous bleeding. In some embodiments, 75 IU/kg of the rVWF is administered twice per week for prophylactic treatment of spontaneous bleeding. In some embodiments, 80 IU/kg of the rVWF is administered twice per week for prophylactic treatment of spontaneous bleeding.

In some embodiments, 40-80 IU/kg rVWF of the rVWF is administered three times per week for prophylactic treatment of spontaneous bleeding. In some embodiments, 50-80 IU/kg of the rVWF is administered three times per week for prophylactic treatment of spontaneous bleeding. In some embodiments, 40 IU/kg rVWF of the rVWF is administered three times per week for prophylactic treatment of spontaneous bleeding. In some embodiments, 45 IU/kg of the rVWF is administered three times per week for prophylactic treatment of spontaneous bleeding. In some embodiments, 50 IU/kg of the rVWF is administered three times per week for prophylactic treatment of spontaneous bleeding. In some embodiments, 55 IU/kg of the rVWF is administered three times per week for prophylactic treatment of spontaneous bleeding. In some embodiments, 60 IU/kg of the rVWF is administered three times per week for prophylactic treatment of spontaneous bleeding. In some embodiments, 65 IU/kg of the rVWF is administered three times per week for prophylactic treatment of spontaneous bleeding. In some embodiments, 70 IU/kg of the rVWF is administered three times per week for prophylactic treatment of spontaneous bleeding. In some embodiments, 75 IU/kg of the rVWF is administered three times per week for prophylactic treatment of spontaneous bleeding. In some embodiments, 80 IU/kg of the rVWF is administered three times per week for prophylactic treatment of spontaneous bleeding. In some embodiments, 50 IU/kg of the rVWF is administered at least every 72 hours for prophylactic treatment of spontaneous bleeding.

In some embodiments, 40-80 IU/kg rVWF of the rVWF is administered once every 2 or 3 days for prophylactic treatment of spontaneous bleeding. In some embodiments, 50-80 IU/kg of the rVWF is administered once every 2 or 3 days for prophylactic treatment of spontaneous bleeding. In some embodiments, 40 IU/kg rVWF of the rVWF is administered once every 2 or 3 days for prophylactic treatment of spontaneous bleeding. In some embodiments, 45 IU/kg of the rVWF is administered once every 2 or 3 days for prophylactic treatment of spontaneous bleeding. In some embodiments, 50 IU/kg of the rVWF is administered once every 2 or 3 days for prophylactic treatment of spontaneous bleeding. In some embodiments, 55 IU/kg of the rVWF is administered once every 2 or 3 days for prophylactic treatment of spontaneous bleeding. In some embodiments, 60 IU/kg of the rVWF is administered once every 2 or 3 days for prophylactic treatment of spontaneous bleeding. In some embodiments, 65 IU/kg of the rVWF is administered once every 2 or 3 days for prophylactic treatment of spontaneous bleeding. In some embodiments, 70 IU/kg of the rVWF is administered once every 2 or 3 days for prophylactic treatment of spontaneous bleeding. In some embodiments, 75 IU/kg of the rVWF is administered once every 2 or 3 days for prophylactic treatment of spontaneous bleeding. In some embodiments, 80 IU/kg of the rVWF is administered once every 2 or 3 days for prophylactic treatment of spontaneous bleeding.

In some embodiments, 40-80 IU/kg of the rVWF is administered once every 3 or 4 days for prophylactic treatment of spontaneous bleeding. In some embodiments, 50-80 IU/kg of the rVWF is administered once every 3 or 4 days for prophylactic treatment of spontaneous bleeding. In some embodiments, 40 IU/kg of the rVWF is administered once every 3 or 4 days for prophylactic treatment of spontaneous bleeding. In some embodiments, 45 IU/kg of the rVWF is administered once every 3 or 4 days for prophylactic treatment of spontaneous bleeding. In some embodiments, 50 IU/kg of the rVWF is administered once every 3 or 4 days for prophylactic treatment of spontaneous bleeding. In some embodiments, 55 IU/kg of the rVWF is administered once every 3 or 4 days for prophylactic treatment of spontaneous bleeding. In some embodiments, 60 IU/kg of the rVWF is administered once every 3 or 4 days for prophylactic treatment of spontaneous bleeding. In some embodiments, 65 IU/kg of the rVWF is administered once every 3 or 4 days for prophylactic treatment of spontaneous bleeding. In some embodiments, 70 IU/kg of the rVWF is administered once every 3 or 4 days for prophylactic treatment of spontaneous bleeding. In some embodiments, 75 IU/kg of the rVWF is administered once every 3 or 4 days for prophylactic treatment of spontaneous bleeding. In some embodiments, 80 IU/kg of the rVWF is administered once every 3 or 4 days for prophylactic treatment of spontaneous bleeding.

In some embodiments, 40-80 IU/kg of the rVWF is administered once either on day 1 and day 5, on day 2 and day 6, or on day 3 and day 7 of a 7 day (week) period for prophylactic treatment of spontaneous bleeding. In some embodiments, 50-80 IU/kg of the rVWF is administered once either on day 1 and day 5, on day 2 and day 6, or on day 3 and day 7 of a 7 day (week) period for prophylactic treatment of spontaneous bleeding. In some embodiments, 40 IU/kg of the rVWF is administered once either on day 1 and day 5, on day 2 and day 6, or on day 3 and day 7 of a 7 day (week) period for prophylactic treatment of spontaneous bleeding. In some embodiments, 45 IU/kg of the rVWF is administered once either on day 1 and day 5, on day 2 and day 6, or on day 3 and day 7 of a 7 day (week) period for prophylactic treatment of spontaneous bleeding. In some embodiments, 50 IU/kg of the rVWF is administered once either on day 1 and day 5, on day 2 and day 6, or on day 3 and day 7 of a 7 day (week) period for prophylactic treatment of spontaneous bleeding. In some embodiments, 55 IU/kg rVWF of the rVWF is administered once either on day 1 and day 5, on day 2 and day 6, or on day 3 and day 7 of a 7 day (week) period for prophylactic treatment of spontaneous bleeding. In some embodiments, 60 IU/kg of the rVWF is administered once either on day 1 and day 5, on day 2 and day 6, or on day 3 and day 7 of a 7 day (week) period for prophylactic treatment of spontaneous bleeding. In some embodiments, 65 IU/kg of the rVWF is administered once either on day 1 and day 5, on day 2 and day 6, or on day 3 and day 7 of a 7 day (week) period for prophylactic treatment of spontaneous bleeding. In some embodiments, 70 IU/kg of the rVWF is administered once either on day 1 and day 5, on day 2 and day 6, or on day 3 and day 7 of a 7 day (week) period for prophylactic treatment of spontaneous bleeding. In some embodiments, 75 IU/kg of the rVWF is administered once either on day 1 and day 5, on day 2 and day 6, or on day 3 and day 7 of a 7 day (week) period for prophylactic treatment of spontaneous bleeding. In some embodiments, 80 IU/kg of the rVWF is administered once either on day 1 and day 5, on day 2 and day 6, or on day 3 and day 7 of a 7 day (week) period for prophylactic treatment of spontaneous bleeding.

In some embodiment, the subject receives prophylactic treatment of spontaneous bleeding for at least a month. In some embodiment, the subject receives prophylactic treatment of spontaneous bleeding for at least 6 months. In some embodiment, the subject receives prophylactic treatment of spontaneous bleeding for at least 1 year. In some embodiment, the subject receives prophylactic treatment of spontaneous bleeding for at least 2 years. In some embodiment, the subject receives prophylactic treatment of spontaneous bleeding for at least 5 years. In some embodiment, the subject receives prophylactic treatment of spontaneous bleeding for at least 10 years.

In some embodiments, a subject with severe VWD is administered a weekly dose of rVWF for prophylactic treatment of spontaneous bleeding episodes. In some embodiments, a weekly dose of rVWF that is substantially equivalent to a weekly dose of plasma-derived VWF (pdVWF) is administered to the subject for prophylaxis of spontaneous bleeding. In some embodiments, a weekly dose of rVWF that is functionally equivalent to a weekly dose of plasma-derived VWF (pdVWF) is administered to the subject for prophylaxis of spontaneous bleeding. In some embodiments, a weekly dose of rVWF that is about 10% less than a weekly dose of plasma-derived VWF (pdVWF) is administered to the subject for prophylaxis of spontaneous bleeding. In some embodiments, a weekly dose of rVWF that is about 10% more than a weekly dose of plasma-derived VWF (pdVWF) is administered to the subject for prophylaxis of spontaneous bleeding.

In some embodiments, a weekly dose of rVWF is administered as at least two separate doses or at least 2 infusions. In some embodiments, a subject is provided at least two doses per week. In some embodiments, a weekly dose of rVWF is administered as 2 infusions. In some embodiments, a weekly dose of rVWF is administered as 2 intravenous infusions. In some cases, the subject is administered a twice-weekly dose of rVWF or in other words, the subject is administered rVWF twice a week. In some embodiments, a weekly dose of rVWF is administered as 2 intravenous infusions. In some embodiments, rVWF is administered to the subject twice a week. In some embodiments, rVWF is administered to the subject every 3 or 4 days. In some embodiments, a first infusion of rVWF is administered and a second infusion of rVWF is administered 4 days later over a 7-day period. In some embodiments, rVWF is administered once on day 1 and day 5 of a 7-day period (a week) for prophylactic treatment of spontaneous bleeding. In some embodiments, a subject is administered a weekly dose that is divided into two infusions such that the subject receives a first infusion on day 1 and second infusion on day 5 of the week period. In some embodiments, rVWF is administered once on day 2 and day 6 of a week period for prophylactic treatment of spontaneous bleeding. In some embodiments, a subject is administered a weekly dose that is divided into two infusions such that the subject receives a first infusion on day 2 and second infusion on day 6 of the week period. In some embodiments, rVWF is administered once on day 3 and day 7 of a week period for prophylactic treatment of spontaneous bleeding. In some embodiments, a subject is administered a weekly dose that is divided into two infusions such that the subject receives a first infusion on day 3 and second infusion on day 7 of the week period. In some embodiments, a weekly dose of rVWF is up to 80 IU/kg of rVWF per each infusion. In some embodiments, a weekly dose of rVWF is divided into 2 intravenous infusions of up to 80 IU/kg of rVWF per each infusion.

In some embodiments, a weekly dose of rVWF is administered as at least 3 separate doses or at least 3 infusions. In some embodiments, a subject is provided at least 3 doses per week. In some embodiments, a weekly dose of rVWF is administered as 3 intravenous infusions. In some cases, the subject is administered a thrice-weekly dose of rVWF or in other words, the subject is administered rVWF three times a week. In some embodiments, rVWF is administered to the subject three times a week. In some embodiments, a weekly dose of rVWF is administered as 3 intravenous infusions such that each infusion is administered on a different day. In some embodiments, rVWF is administered to the subject every 2 or 3 days. In some embodiments, a first infusion of rVWF is administered, a second infusion of rVWF is administered 2 days later, and a third infusion of rVWF is administered 3 days later over a 7-day period. In some embodiments, rVWF is administered once on day 1, day 3, and day 6 of a 7-day period for prophylactic treatment of spontaneous bleeding. In some embodiments, a subject is administered a weekly dose that is divided into 3 infusions such that the subject receives a first infusion on day 1, second infusion on day 3, a third infusion on day 6 of the week period. In some embodiments, rVWF is administered once on day 1, day 3, and day 6 of a week period for prophylactic treatment of spontaneous bleeding. In some embodiments, a subject is administered a weekly dose that is divided into 3 infusions such that the subject receives a first infusion on day 2, second infusion on day 4, a third infusion on day 7 of the week period. In some embodiments, rVWF is administered once on day 2, day 4, and day 7 of a week period for prophylactic treatment of spontaneous bleeding. In some embodiments, a weekly dose of rVWF is up to 80 IU/kg of rVWF per each infusion. In some embodiments, a weekly dose of rVWF is divided into 3 intravenous infusions of up to 80 IU/kg of rVWF per each infusion.

In some embodiments, a weekly dose of rVWF is administered as a single dose or a single infusion. In some embodiments, a weekly dose of rVWF is administered as a single infusion. In some embodiments, a weekly dose of rVWF is administered as a single intravenous infusion. In some embodiments, a subject is administered a once-weekly dose of rVWF if the subject has previously received a once-weekly dose of pdVWF. In some embodiments, a weekly dose of rVWF is up to 80 IU/kg of rVWF for such an infusion.

In some embodiments, the subject experiences at least a 5% reduction (e.g., 5%, 7%, 10%, 12%, 14%, 15%, 17%, 20%, 22%, 24%, 25%, 27%, 30%, 32%, 34%, 35%, 37%, 40%, 42%, 44%, 45%, 47%, 50% or more reduction) in annual bleeding rate (ABR) for spontaneous bleeding events upon receiving the prophylactic treatment described herein. In some embodiments, the subject experiences at least a 25% reduction (e.g., 25%, 27%, 30%, 35%, 40%, 45%, 50%, 55%, 60%, 65%, 70%, 75%, 80%, 85%, 90% or more reduction) in ABR for spontaneous bleeding events upon receiving the prophylactic treatment. In some embodiments, a reduction is the number of spontaneous bleeding episodes is indicated by a reduction in the annual bleeding rate (ABR). In some embodiments, the pretreatment ABR is determined based on the following formula: number of bleeds/days not on treatment regimen. In some embodiments, the pretreatment ABR is determined based on the following formula: number of bleeds/12 months prior to the prophylactic treatment with rVWF. In some embodiments, the prophylactic ABR (ABR after prophylactic treatment) is determined based on the following formula: number of bleeds/days on treatment regimen.

In some embodiments, the subject experiences no spontaneous bleeding events during the course of prophylactic treatment with rVWF. In some embodiments, the subject experiences 1-2 spontaneous bleeding events during the course of prophylactic treatment with rVWF. In some embodiments, the subject experiences 1-3 spontaneous bleeding events during the course of prophylactic treatment with rVWF. In some embodiments, the subject experiences 3-5 spontaneous bleeding events during the course of prophylactic treatment with rVWF. In some embodiments, the subject experiences 1-5 spontaneous bleeding events during the course of prophylactic treatment with rVWF. In some embodiments, the subject experiences more than 5 spontaneous bleeding events during the course of prophylactic treatment with rVWF.

In some embodiments, administration of rVWF reduces the number of spontaneous bleeding events to 0 per year. In some embodiments, administration of rVWF reduces the number of spontaneous bleeding events to 1 per year. In some embodiments, administration of rVWF reduces the number of spontaneous bleeding events to 2 per year. In some embodiments, administration of rVWF reduces the number of spontaneous bleeding events to 3 per year. In some embodiments, administration of rVWF reduces the number of spontaneous bleeding events to 4 per year. In some embodiments, administration of rVWF reduces the number of spontaneous bleeding events to 5 per year. In some embodiments, administration of rVWF reduces the number of spontaneous bleeding events to more than 5 per year. In some embodiments, administration of rVWF reduces the number of spontaneous bleeding events to 1-5 per year. In some embodiments, administration of rVWF reduces the number of spontaneous bleeding events to 3-5 per year. In some embodiments, administration of rVWF reduces the number of spontaneous bleeding events to 1-3 per year. In some embodiments, administration of rVWF reduces the number of spontaneous bleeding events to less than 5 per year. In some embodiments, administration of rVWF reduces the number of spontaneous bleeding events to less than 4 per year. In some embodiments, administration of rVWF reduces the number of spontaneous bleeding events to less than 3 per year. In some embodiments, administration of rVWF reduces the number of spontaneous bleeding events to less than 2 per year. In some embodiments, administration of rVWF reduces the number of spontaneous bleeding events to less than 1 per year.

In some embodiments, prophylactic treatment reduces or diminishes the severity (e.g., mild, moderate, and severe bleeding episodes), incidence, frequency, or duration of a spontaneous bleeding episode. In some embodiments, the subject administered rVWF has fewer spontaneous bleeding episodes than a control subject that has not received prophylactic treatment. In some embodiments, the subject administered rVWF has fewer spontaneous bleeding episodes over the course of treatment compared to prior to initiating treatment.

Generally, Type 1 VWD is indicated by <30 IU/dL VWF:RCo, <30 IU/dL VWF:Ag, low or normal FVIII, and >0.5-0.7 IU/dL VWF:RCo/VWF:Ag Ratio. In some embodiments, a subject diagnosed with Type 1 VWD has <20 IU/dL VWF:RCo. Type 2A VWD is indicated by <30 IU/dL VWF:RCo, <30-200 IU/dL VWF:Ag, low or normal FVIII, and <0.5-0.7 IU/dL VWF:RCo/VWF:Ag Ratio. Type 2B VWD is indicated by <30-200 IU/dL VWF:RCo, <30 IU/dL VWF:Ag, low or normal FVIII, and usually <0.5-0.7 IU/dL VWF:RCo/VWF:Ag Ratio. Type 2M VWD is indicated by <30 IU/dL VWF:RCo, <30-200 IU/dL VWF:Ag, low or normal FVIII, and <0.5-0.7 IU/dL VWF:RCo/VWF:Ag Ratio. Type 2N VWD is indicated by 30-2000 IU/dL VWF:RCo, 30-200 IU/dL VWF:Ag, very low FVIII, and >0.5-0.7 IU/dL VWF:RCo/VWF:Ag Ratio. Type 3 VWD is indicated by <3 IU/dL VWF:RCo, <3 IU/dL VWF:Ag, extremely low (<10 IU/dL) FVIII, and the VWF:RCo/VWF:Ag Ratio is not applicable. In some embodiments, a subject diagnosed with Type 3 VWD has <3 IU/dL VWF:Ag. Normal is indicated by 50-200 IU/dL VWF:RCo, 50-200 IU/dL VWF:Ag, normal FVIII, and >0.5-0.7 IU/dL VWF:RCo/VWF:Ag Ratio. In some embodiments, the subject has Type 3 VWD. In some embodiments, the subject has severe type 1 VWD. In some embodiments, the subject has severe type 2 VWD. In some embodiments, the subject has severe type 2A VWD. In some embodiments, the subject has severe type 2B VWD. In some embodiments, the subject has severe type 2M VWD. In some embodiments, the subject has severe type 3 VWD.

In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with Type 1 VWD. In some embodiments, an improvement in FVIII, VWF:RCo, and/or VWF:Ag activity levels includes a change in the activity levels such that the activity levels are closer to normal levels, levels in a subject that does not have Type 1 VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with severe Type 1 VWD. In some embodiments, an improvement in FVIII, VWF:RCo, and/or VWF:Ag activity levels includes a change in the activity levels such that the activity levels are closer to normal levels, e.g., levels in a subject that does not have severe Type 1 VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with Type 2A VWD. In some embodiments, an improvement in FVIII, VWF:RCo, and/or VWF:Ag activity levels includes a change in the activity levels such that the activity levels are closer to normal levels, e.g., levels in a subject that does not have Type 2A VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with severe Type 2A VWD. In some embodiments, an improvement in FVIII, VWF:RCo, and/or VWF:Ag activity levels includes a change in the activity levels such that the activity levels are closer to normal levels, e.g., levels in a subject that does not have severe Type 2A VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with Type 2M VWD. In some embodiments, an improvement in FVIII, VWF:RCo, and/or VWF:Ag activity levels includes a change in the activity levels such that the activity levels are closer to normal levels, e.g., levels in a subject that does not have Type 2M VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with severe Type 2M VWD. In some embodiments, an improvement in FVIII, VWF:RCo, and/or VWF:Ag activity levels includes a change in the activity levels such that the activity levels are closer to normal levels; e.g., levels in a subject that does not have severe Type 2M VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with Type 2N VWD. In some embodiments, an improvement in FVIII, VWF:RCo, and/or VWF:Ag activity levels includes a change in the activity levels such that the activity levels are closer to normal levels, e.g., levels in a subject that does not have Type 2N VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with severe Type 2N VWD. In some embodiments, an improvement in FVIII, VWF:RCo, and/or VWF:Ag activity levels includes a change in the activity levels such that the activity levels are closer to normal levels, e.g., levels in a subject that does not have severe Type 2N VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with Type 3 VWD. In some embodiments, an improvement in FVIII, VWF:RCo, and/or VWF:Ag activity levels includes a change in the activity levels such that the activity levels are closer to normal levels, e.g., levels in a subject that does not have Type 3 VWD. In some embodiments, prophylactic treatment efficacy is indicated by an improvement in FVIII, VWF:RCo, and/or VWF:Ag activity levels after the prophylactic treatment with rVWF as compared to the levels prior to the prophylactic treatment with rVWF in patients with severe Type 3 VWD. In some embodiments, an improvement in FVIII, VWF:RCo, and/or VWF:Ag activity levels includes a change in the activity levels such that the activity levels are closer to normal levels, e.g., levels in a subject that does not have severe Type 3 VWD. In some embodiments, an improvement in FVIII, VWF:RCo, and/or VWF:Ag activity levels includes a change in the activity levels such that the activity levels are closer to normal levels, e.g., levels in a subject that does not have any form of VWD.

In some embodiments, the subject has received a diagnosis of VWD. In some instance, the VWD diagnosis is confirmed by genetic testing, multimer analysis, patient history, or a combination thereof.

In some embodiments, the subject had been treated for at least 1 spontaneous bleeding event within the previous 12 months. In some embodiments, the subject had been treated for more than 1 spontaneous bleeding event within the previous 12 months. In some embodiments, the subject had been treated for at least 3 spontaneous bleeding event within the previous 12 months. In some embodiments, the subject had been treated for 3 or more spontaneous bleeding events within the previous 12 months.

In some embodiments, the subject is currently receiving on-demand treatment for a spontaneous bleeding event (e.g., episode). In some embodiments, the subject is currently receiving VWF therapy (e.g., rVWF or pdVWF therapy) to treat a spontaneous bleeding event. In some embodiments, the subject is not currently receiving on-demand treatment for a spontaneous bleeding event (e.g., episode). In some embodiments, the subject is not currently receiving VWF therapy (e.g., rVWF or pdVWF therapy) to treat a spontaneous bleeding event.

In some embodiments, the subject has been prophylactically treated for spontaneous bleeding by administration of pdVWF within the previous 12 months. In some embodiments, the subject has been prophylactically treated for spontaneous bleeding by administration of pdVWF for the previous 12 months. In some embodiments, the subject has been prophylactically treated for spontaneous bleeding by administration of pdVWF for at least the previous 12 months. In some embodiments, the subject has not been prophylactically treated for spontaneous bleeding by administration of pdVWF within the previous 12 months. In some embodiments, the subject has not been prophylactically treated for spontaneous bleeding by administration of pdVWF for the previous 12 months. In some embodiments, the subject has not been prophylactically treated for spontaneous bleeding by administration of pdVWF for at least the previous 12 months.

Generally, minor bleeding is characterized by acute or subacute clinically overt bleeding that did not satisfy the criteria for major bleeding and led to hospital admission for bleeding, physician-guided medical or surgical treatment for bleeding, or a change in antithrombotic therapy (including study drugs) for bleeding (Aristotle clinical definition); All other bleeding (except major and ICH) (RE-LY clinical definition); Overt bleeding not meeting the criteria for major bleeding but requiring medical intervention, unscheduled contact (visit or telephone) with a physician, temporary interruption of study drug (i.e., delayed dosing), pain, or impairment of daily activities) Rocket-AF clinical definition); Clinically relevant bleeding was defined as skin hematoma >25 cm², spontaneous nosebleed of >5 minutes duration, macroscopic hematuria, spontaneous rectal bleeding, gingival bleeding for >5 minutes, any bleeding leading to hospitalization, any bleeding leading to transfusion <2 U, or any other bleeding considered relevant by the investigator (Petro clinical definition); and/or CRNM (clinically relevant non-major bleeding) defined as acute or subacute, clinically overt, not major, and leading to hospital admission for bleeding, physician-guided medical or surgical treatment for bleeding, or a change in antithrombotic therapy as well as minor bleeding events defined as acute clinically overt events not meeting the criteria for either major or CRNM bleeding (Aristotle-J clinical definition). See, for example, Wells G, Coyle D, Cameron C, et al. Safety, Effectiveness, and Cost-Effectiveness of New Oral Anticoagulants Compared with Warfarin in Preventing Stroke and Other Cardiovascular Events in Patients with Atrial Fibrillation [Internet]. Ottawa (ON): Canadian Agency for Drugs and Technologies in Health; 2012 Apr. 9. 3, CLINICAL REVIEW. Available on the World Wide Web at www.ncbi.nlm.nih.gov/books/NBK169813/. Minor bleeding can include events were defined as those not fulfilling the criteria of major or clinically significant bleeding; minor bleeding from a wound (bleeding at the injection site, epistaxis, or wound hematoma not requiring operative decompression); overt bleeding that did not meet the criteria for major hemorrhage and associated with ≥1 of the following: epistaxis lasting more than 5 minutes or requiring intervention, ecchymosis or hematoma ≥5 cm at its greatest dimension, hematuria not associated with urinary catheter related trauma, GI hemorrhage not related to intubation or placement of a NG tube, wound hematoma or complications, subconjunctival hemorrhage necessitating cessation of medication; minor bleeding in the GI or urinary tract and hematoma at the site of an injection; and/or overt bleeding not meeting the criteria for major hemorrhage. See, for example, Sobieraj D M, Coleman C I, Tongbram V, et al. Venous Thromboembolism Prophylaxis in Orthopedic Surgery [Internet]. Rockville (Md.): Agency for Healthcare Research and Quality (US); 2012 March (Comparative Effectiveness Reviews, No. 49.) Appendix F, Additional Evidence Tables. Available from the World Wide Web at www.ncbi.nlm.nih.gov/booksNBK92309/.

Generally major bleeding is characterized by International Society on Thrombosis and Haemostasis (ISTH) standards, and includes, any life threatening and/or fatal bleeding; symptomatic bleeding into a critical area or organ and major bleeding was separated into intracranial (intracerebral, subdural) and extracranial (GI, non-GI) bleeding (RE-LY clinical definition); symptomatic bleeding into a critical anatomic site (Rocket-AF clinical definition); Life-threatening retroperitoneal, intracranial, intraocular, or intraspinal bleeding; or bleeding requiring surgery (Artistotle-J clinical definition). Major bleeding events can include those where there is fall in hemoglobin at least 20 g/L or transfusion of >2 units of whole blood (packed cells mentioned in life-threatening bleed definition; RE-LY definition of life-threatening bleeding: ≥1 of the following criteria: (1) fatal, symptomatic intracranial bleed; (2) reduction in hemoglobin level of at least 5.0 g/L; (3) transfusion of at least 4 U of blood or packed cells; (4) associated with hypotension requiring the use of intravenous inotropic agents; or (5) necessitated surgical intervention); fall in hemoglobin >2 g/dL or transfusion of >2 units of whole blood/red cells (ISTH or Rocket-AF clinical definition); and/or bleeding requiring surgery or transfusion of ≥2 U or associated with a decrease in hemoglobin of ≥2.0 g/L episodes. See, for example, Wells G, Coyle D, Cameron C, et al. Safety, Effectiveness, and Cost-Effectiveness of New Oral Anticoagulants Compared with Warfarin in Preventing Stroke and Other Cardiovascular Events in Patients with Atrial Fibrillation [Internet]. Ottawa (ON): Canadian Agency for Drugs and Technologies in Health; 2012 Apr. 9. 3, CLINICAL REVIEW. Available on the World Wide Web at www.ncbi.nlm.nih.gov/books/NBK169813/. Major bleeding can include clinically overt bleeding associated with >20 g/L fall in Hb; clinically overt leading to transfusion of >2 U packed cells or whole blood; fatal, retroperitoneal, intracranial, intraocular or intraspinal bleeding; bleeding warranting treatment cessation or leading to reoperation; fatal, retroperitoneal, intracranial, or intraspinal bleeding; bleeding that involved any other critical organ; bleeding leading to reoperation; overt bleeding with a bleeding index ≥2; major bleeding from a wound (wound hematoma requiring operative decompression), or major bleeding not related to a wound (gastrointestinal or intracerebral hemorrhage); clinically overt bleeding associated with either a decrease in Hb ≥2 g/dL or a need for a transfusion of ≥2 U RBC; intracranial or retroperitoneal (resulted in the permanent discontinuation of anticoagulation). See, for example, Sobieraj D M, Coleman C I, Tongbram V, et al. Venous Thromboembolism Prophylaxis in Orthopedic Surgery [Internet]. Rockville (Md.): Agency for Healthcare Research and Quality (US); 2012 March (Comparative Effectiveness Reviews, No. 49.) Appendix F, Additional Evidence Tables. Available from: the World Wide Web at www.ncbi.nlm.nih.gov/books/NBK92309/.

Compositions of rVWF can be contained in pharmaceutical formulations, as described herein. Such formulations can be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. In some embodiments, the rVWF is administered intravenously. Administration by intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

In one aspect, formulations of the invention are administered by an initial bolus followed by a continuous infusion to maintain therapeutic circulating levels of drug product. As another example, the inventive compound is administered as a one-time dose. Those of ordinary skill in the art will readily optimize effective dosages and administration regimens as determined by good medical practice and the clinical condition of the individual patient. The route of administration can be, but is not limited to, by intravenous, intraperitoneal, subcutaneous, or intramuscular administration. The frequency of dosing depends on the pharmacokinetic parameters of the agents and the route of administration. The optimal pharmaceutical formulation is determined by one skilled in the art depending upon the route of administration and desired dosage. See for example, Remington's Pharmaceutical Sciences, 18th Ed., 1990, Mack Publishing Co., Easton, Pa. 18042 pages 1435-1712, the disclosure of which is hereby incorporated by reference in its entirety for all purposes and in particular for all teachings related to formulations, routes of administration and dosages for pharmaceutical products. Such formulations influence the physical state, stability, rate of in vivo release, and rate of in vivo clearance of the administered agents. Depending on the route of administration, a suitable dose is calculated according to body weight, body surface area or organ size. Appropriate dosages may be ascertained through use of established assays for determining blood level dosages in conjunction with appropriate dose-response data. The final dosage regimen is determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. By way of example, a typical dose of a recombinant VWF of the present invention is approximately 50 IU/kg, equal to 500 μg/kg. As studies are conducted, further information will emerge regarding the appropriate dosage levels and duration of treatment for various diseases and conditions.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, Highly stabilized York, Gait, “Oligonucleotide Synthesis: A Practical Approach” 1984, IRL Press, London, Nelson and Cox (2000), Lehninger, Principles of Biochemistry 3rd Ed., W. H. Freeman Pub., Highly stabilized York, N.Y. and Berg et al. (2002) Biochemistry, 5th Ed., W. H. Freeman Pub., Highly stabilized York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “an antibody” includes a plurality of such antibodies and reference to “a host cell” includes reference to one or more host cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the above description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive method.

### EXAMPLES

### Example 1: Efficacy and Safety of Prophylaxis with Recombinant Von Willebrand Factor (VWF) in Severe Von Willebrand Disease (VWD): Design of a Prospective, Phase 3, Open-Label, International Multicenter Study

### Introduction

Patients with von Willebrand disease (VWD) have impaired hemostasis owing to a quantitative or qualitative deficit in von Willebrand Factor (VWF), a large multimeric plasma glycoprotein with key hemostatic functions mediating platelet aggregation and stabilizing blood clotting factor VIII (FVIII) in circulation (1-3). Most patients with VWD present with mild to moderate mucosal bleeding and bleeding after trauma or surgery, although life-threatening bleeding may also occur, particularly in patients with severe disease (1,2).

Recombinant VWF (rVWF, vonicog alfa, VEYVONDI™; Baxalta Innovations GmbH, a Takeda company, Vienna, Austria) is manufactured by recombinant DNA technology in the Chinese hamster ovary cell line without the addition of exogenous human or animal-derived protein (4). VWF contains the full VWF multimer profile, including ultra-large multimers that are usually deficient in plasma-derived VWF (pdVWF) concentrates exposed to ADAMTS13, the VWF-cleaving protease (5-7)

Patients with severe VWD may benefit from prophylactic rVWF treatment to maintain VWF and FVIII levels so that the risk of spontaneous bleeding episodes (BEs), including hemarthrosis, epistaxis, and gastrointestinal bleeding, is reduced (8-13).

Reducing the frequency and duration of BEs likely decreased the need for red blood cell transfusions and lower the risk of debilitating comorbidities, including arthropathy (8,9)

### Study Objectives

The objective of the study was to investigate the efficacy and safety of long-term prophylactic treatment with rVWF in patients with severe VWD.

### Study Design

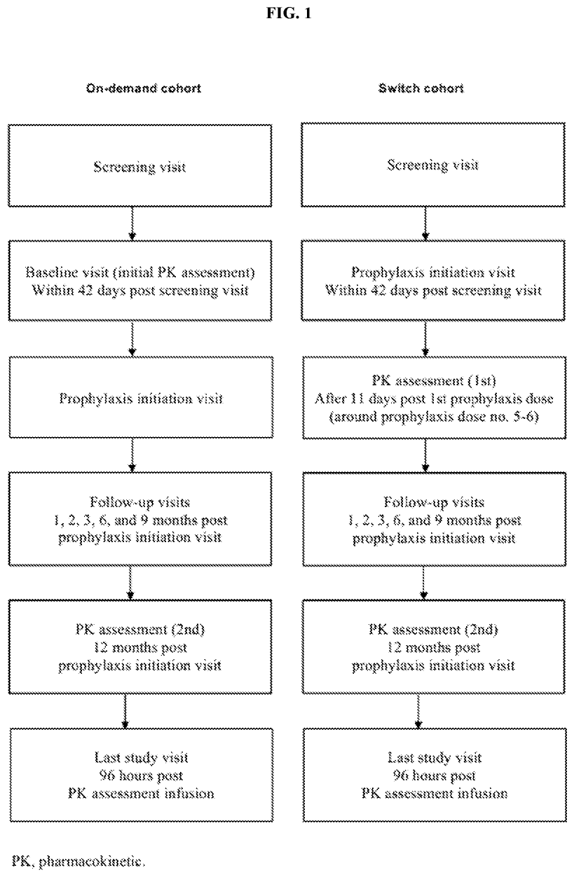

Described herein is a global, multicenter, open-label, phase 3 study (NCT02973087, EudraCT no.: 2016-001478-14) to prospectively evaluate the efficacy, safety, and pharmacokinetics of continuous prophylactic treatment with rVWF in adult patients with severe VWD (baseline VWF ristocetin cofactor activity [VWF:RCo]<20 IU/dL) who have required therapy with pdVWF to control BEs in the year prior to enrollment (FIG. 1).

Patients belong to 1 or 2 cohorts, depending on the type of VWF regimen received prior to enrollment in the study: on-demand treatment (on-demand cohort) and prophylactic treatment with a pdVWF product (switch cohort). Patients in both cohorts to receive prophylactic rVWF treatment for 1 year.

### Patients

Patients ≥18 years of age diagnosed with severe VWD (baseline VWF:RCo <20 IU/dL) requiring VWF treatment to control bleeding (key study eligibility criteria are detailed in FIG. 2) The study protocol was approved by the relevant local ethics committee and all patients will have provided written informed consent before enrollment.

### Study Treatment

Patients received rVWF prophylaxis as described below.

For the on-demand cohort, a standard prophylactic dose of 50±10 IU/kg rVWF:RCo was administered by intravenous infusion. The dose could be increased up to 80 IU/kg.

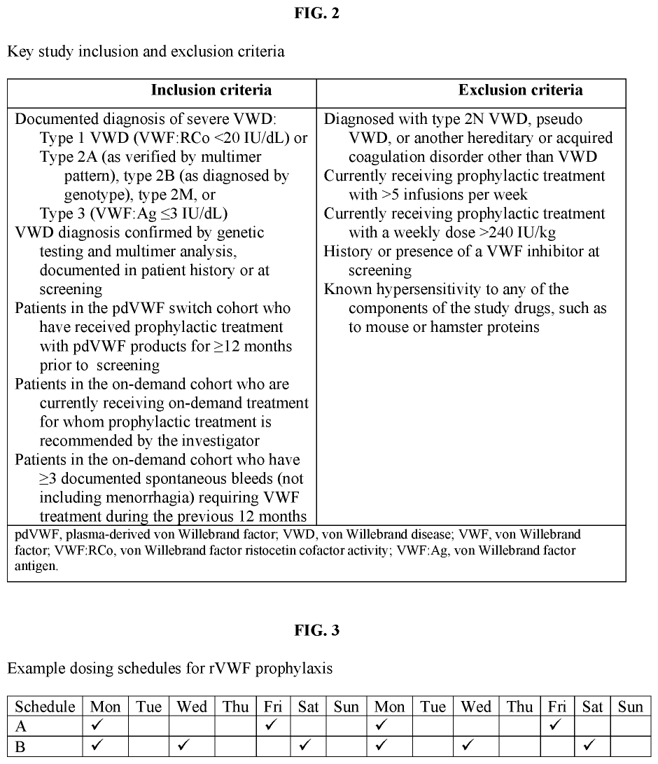

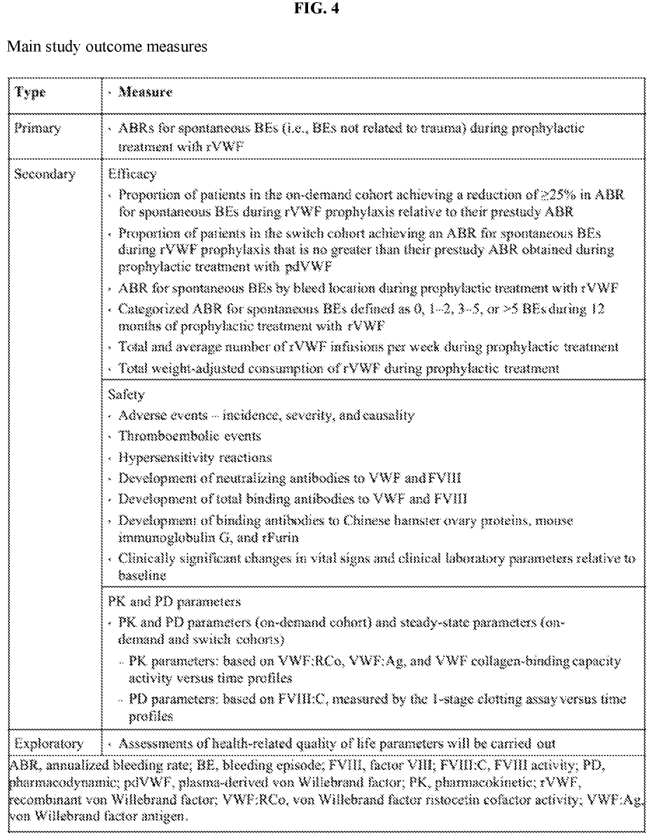

All patients were initiated on twice-weekly dosing (FIG. 3, Schedule A).

For the switch cohort, a weekly rVWF dose equivalent to ±10% of the weekly pdVWF dose received previously, was divided into 2 intravenous infusions (FIG. 3, Schedule A), to a maximum of 80 IU/kg per infusion. The weekly dose was given as 3 infusions (FIG. 3, Schedule B), according to clinical judgment; once-weekly dosing was only permitted if the patient had received prior once-weekly dosing with pdVWF.

The rVWF dose could be further individualized with the specified range on the basis of pharmacokinetic data, history of BEs, and the results from clinical and laboratory assessments. Patients experiencing BEs requiring VWF treatment would have received rVWF to treat the bleed. Weight-adjusted dosing should be based on the type and severity of the BE and appropriate clinical and laboratory measures. Recombinant FVIII (antihemophilic factor [recombinant] [ADVATE; Baxalta US Inc., a Takeda company, Lexington, Mass.]) would have been administered if an immediate increase of the FVIII level is clinically indicated.

Patients requiring elective surgery or dental procedures during the study were treated with rVWF to manage surgical bleeding and then resumed their prophylactic rVWF treatment schedule. Before surgery, rVWF was administered without recombinant FVIII if FVIII activity (FVIII:C) levels are at the recommended target level of: ≥0.4 IU/mL for minor or oral surgery, and ≥0.8 IU/mL for major surgery.

### Study Outcome Measures and Statistical Analysis

The main study outcome measures are listed in FIG. 4 (Table 3).

Statistical analysis included approximately 22 patients, including at least 5 patients with type 3 VWD and at least 8 patients in each study cohort; no formal sample size calculations were done.

The primary efficacy analysis was conducted on all patients who have received prophylactic rVWF treatment. Spontaneous ABRs will be estimated using a negative binominal regression for each study cohort (on-demand cohort or switch cohort). The comparison of prestudy and on-study spontaneous ABRs was based on the patients in the switch cohort. Assessment will use a generalized linear mixed-effects model. ABR ratio was reported with a 2-sided 95% confidence interval for each study cohort.

### Study Status and Conclusion

Study enrollment is now completed.

Results from this prospective phase 3 study have provided data on the efficacy and safety of rVWF for prophylaxis against spontaneous bleeds in patients with severe VWD.

The study period of prophylactic rVWF treatment was 1 year, and the primary efficacy outcome measure is the ABR for spontaneous (nontraumatic) bleeds.

### Primary Outcome Measures

Prospectively recorded annualized bleeding rate (ABR) for spontaneous (not related to trauma) bleeding episodes during prophylactic treatment with (rVWF) and the participants' historical ABR for spontaneous bleeding episodes during on-demand treatment (Time Frame: Approximately 1 year).

### Revised Secondary Outcome Measures

Number of participants with reduction of annualized bleeding rate (ABR) for spontaneous (not related to trauma) bleeding episodes during prophylaxis relative to the participants' own historical ABR during on-demand treatment (Time Frame: Approximately 1 year).

Number of participants with zero bleeds during prophylactic treatment with recombinant von Willebrand factor (rVWF) (Time Frame: Approximately 1 year).

Number of infusions of recombinant von Willebrand factor (rVWF) and ADVATE per month during prophylactic treatment as well as during on-demand treatment (Time Frame: Approximately 1 year).

Number of infusions of recombinant von Willebrand factor (rVWF) and ADVATE per year during prophylactic treatment as well as during on-demand treatment (Time Frame: Approximately 1 year).

Total weight adjusted consumption of recombinant von Willebrand factor (rVWF) and ADVATE per month during prophylactic treatment as well as during on-demand treatment (Time Frame: Approximately 1 year).

Total weight adjusted consumption of recombinant von Willebrand factor (rVWF) and ADVATE per year during prophylactic treatment as well as during on-demand treatment (Time Frame: Approximately 1 year).

Incidence of thromboembolic events (Time Frame: Throughout the study period of approximately 22 months).

Incidence of severe hypersensitivity reactions (Time Frame: Throughout the study period of approximately 22 months).

Number of participants who develop neutralizing antibodies to recombinant von Willebrand factor (rVWF) and Factor VIII (FVIII) (Time Frame: Throughout the study period of approximately 22 months).

Number of participants who develop total binding antibodies to recombinant von Willebrand factor (rVWF) and Factor VIII (FVIII) (Time Frame: Throughout the study period of approximately 22 months).

Number of participants who develop antibodies to Chinese hamster ovary (CHO) proteins, mouse immunoglobulin G (IgG) and rFurin (Time Frame: Throughout the study period of approximately 22 months).

Pharmacokinetics—Incremental recovery (IR) (Time Frame: 30 minutes pre-infusion; and post-infusion at 30 minutes and 1, 6, 12, 24. 48, and 72 hours).

Pharmacokinetics—Terminal half-life (T½) (Time Frame: 30 minutes pre-infusion; and post-infusion at 30 minutes and 1, 6, 12, 24. 48, and 72 hours).

Pharmacokinetics—Mean residence time (MRT) (Time Frame: 30 minutes pre-infusion; and post-infusion at 30 minutes and 1, 6, 12, 24. 48, and 72 hours).

Pharmacokinetics—Area under the curve/dose (AUC/dose) (Time Frame: 30 minutes pre-infusion; and post-infusion at 30 minutes and 1, 6, 12, 24. 48, and 72 hours).

Pharmacokinetics—Area under moment curve/dose (AUMC/dose) (Time Frame: 30 minutes pre-infusion; and post-infusion at 30 minutes and 1, 6, 12, 24. 48, and 72 hours).

Pharmacokinetics—Volume of distribution at steady state (Vss) (Time Frame: 30 minutes pre-infusion; and post-infusion at 30 minutes and 1, 6, 12, 24. 48, and 72 hours).

Pharmacokinetics—Clearance (CL) (Time Frame: 30 minutes pre-infusion; and post-infusion at 30 minutes and 1, 6, 12, 24. 48, and 72 hours).

Number of infusions of recombinant von Willebrand factor (rVWF) and ADVATE (rFVIII) per spontaneous bleeding episode (BE) (Time Frame: Throughout the study period, up to approximately 22 months).

Number of infusions of recombinant von Willebrand factor (rVWF) and ADVATE (rFVIII) per traumatic bleeding episode (BE) (Time Frame: Throughout the study period, up to approximately 22 months).

Weight-adjusted consumption of recombinant von Willebrand factor (rVWF) and ADVATE (rFVIII) per spontaneous bleeding episode (BE) (Time Frame: Throughout the study period, up to approximately 22 months).

Weight-adjusted consumption of recombinant von Willebrand factor (rVWF) and ADVATE (rFVIII) per traumatic bleeding episode (BE) (Time Frame: Throughout the study period, up to approximately 22 months).

Overall hemostatic efficacy rating at resolution of bleed (Time Frame: Throughout the study period, up to approximately 22 months). Used a 4-point scale: Excellent, Good, Moderate, None.

Intraoperative actual versus predicted blood loss—if surgery is required (Time Frame: Day 0 (at completion of surgery)). Assessed by the operating surgeon) at completion of surgery.

Intraoperative hemostatic efficacy—if surgery is required (Time Frame: Day 0 (at completion of surgery)). Score on a scale of excellent, good, moderate or none—assessed by the operating surgeon at completion of surgery.

For elective surgery: an overall assessment of hemostatic efficacy 24 hours and on Day 7 and Day 14 after the last perioperative infusion of rVWF (Time Frame: 24 hours and on Day 7 and Day 14 after the last perioperative infusion of rVWF). Score on a scale of excellent, good, moderate or none.

Daily intra- and postoperative weight-adjusted dose (Time Frame: Day 0 (surgery day) through postoperative day 14). Daily intra- and postoperative weight-adjusted dose of rVWF with or without ADVATE.

### Original Secondary Outcome Measures

Number of participants with reduction of annualized bleeding rate (ABR) for spontaneous (not related to trauma) bleeding episodes during prophylaxis relative to the participants' own historical ABR during on-demand treatment (Time Frame: Approximately 1 year).

Number of participants with zero bleeds during prophylactic treatment with recombinant von Willebrand factor (rVWF) (Time Frame: Approximately 1 year).

Number of infusions of recombinant von Willebrand factor (rVWF) and ADVATE per month during on-demand treatment (Time Frame: Approximately 1 year).

Number of infusions of recombinant von Willebrand factor (rVWF) and ADVATE per year during on-demand treatment (Time Frame: Approximately 1 year).

Total weight adjusted consumption of recombinant von Willebrand factor (rVWF) and ADVATE per month on-demand treatment (Time Frame: Approximately 1 year).

Total weight adjusted consumption of recombinant von Willebrand factor (rVWF) and ADVATE per year on-demand treatment (Time Frame: Approximately 1 year).

Incidence of thromboembolic events (Time Frame: Throughout the study period of approximately 22 months).

Incidence of severe hypersensitivity reactions (Time Frame: Throughout the study period of approximately 22 months).

Number of participants who develop neutralizing antibodies to recombinant von Willebrand factor (rVWF) and Factor VIII (FVIII) (Time Frame: Throughout the study period of approximately 22 months).

Number of participants who develop total binding antibodies to recombinant von Willebrand factor (rVWF) and Factor VIII (FVIII) (Time Frame: Throughout the study period of approximately 22 months).

Number of participants who develop antibodies to Chinese hamster ovary (CHO) proteins, mouse immunoglobulin G (IgG) and rFurin (Time Frame: Throughout the study period of approximately 22 months).

Pharmacokinetics—Incremental recovery (IR) (Time Frame: 30 minutes pre-infusion; and post-infusion at 30 minutes and 1, 6, 12, 24. 48, and 72 hours).

Pharmacokinetics—Terminal half-life (T½) (Time Frame: 30 minutes pre-infusion; and post-infusion at 30 minutes and 1, 6, 12, 24. 48, and 72 hours).

Pharmacokinetics—Mean residence time (MRT) (Time Frame: 30 minutes pre-infusion; and post-infusion at 30 minutes and 1, 6, 12, 24. 48, and 72 hours).

Pharmacokinetics—Area under the curve/dose (AUC/dose) (Time Frame: 30 minutes pre-infusion; and post-infusion at 30 minutes and 1, 6, 12, 24. 48, and 72 hours).

Pharmacokinetics—Area under moment curve/dose (AUMC/dose) (Time Frame: 30 minutes pre-infusion; and post-infusion at 30 minutes and 1, 6, 12, 24. 48, and 72 hours).

Pharmacokinetics—Volume of distribution at steady state (Vss) (Time Frame: 30 minutes pre-infusion; and post-infusion at 30 minutes and 1, 6, 12, 24. 48, and 72 hours).

Pharmacokinetics—Clearance (CL) (Time Frame: 30 minutes pre-infusion; and post-infusion at 30 minutes and 1, 6, 12, 24. 48, and 72 hours).

Number of infusions of recombinant von Willebrand factor (rVWF) and ADVATE (rFVIII) per spontaneous bleeding episode (BE) (Time Frame: Throughout the study period, up to approximately 22 months).

Number of infusions of recombinant von Willebrand factor (rVWF) and ADVATE (rFVIII) per traumatic bleeding episode (BE) (Time Frame: Throughout the study period, up to approximately 22 months).

Weight-adjusted consumption of recombinant von Willebrand factor (rVWF) and ADVATE (rFVIII) per spontaneous bleeding episode (BE) (Time Frame: Throughout the study period, up to approximately 22 months).

Weight-adjusted consumption of recombinant von Willebrand factor (rVWF) and ADVATE (rFVIII) per traumatic bleeding episode (BE) (Time Frame: Throughout the study period, up to approximately 22 months).

Overall hemostatic efficacy rating at resolution of bleed (Time Frame: Throughout the study period, up to approximately 22 months). Used a 4-point scale: Excellent, Good, Moderate, None.

### REFERENCES

1. Nichols W L, et al., Haemophilia 2008; 14:171-232.
2. Leebeek F W, Eikenboom J C. N Engl J Med 2016; 375:2067-80.
3. Reininger A J. Hamostaseologie 2015; 35:225-33.
4. European Medicines Agency. VEYVONDI Summary of Product Characteristics. https://www.ema,europa,ed/documents/product-information/veyvondi-epar-product-information_en.pdf.
5. Mannucci P M et al., Blood 1994; 83:3018-27.
6. Turecek P L et al., Hamostaseologie 2009; 20(suppl 1):S32-8.
7. Favaloro E J., Blood Transfus. 2016; 14:262-76.
8. Abshire T., Thromb Res 2009; 124(suppl 1):S15-9.
9. Berntorp E., Haemophilia 2008; 14(suppl 5):47-53.
10. Berntorp E., Petrini P., Blood Coagul Fibrinolysis 2005; 16(suppl 1):523-6.
11. Berntorp E., Semin Thromb Hemost 2006; 32:621-5.
12. Abshire T C, et al., Haemophilia 2013; 19:76-81.
13. Abshire T et al., J Thromb Haemost 2015; 13:1585-9.

### Example 2: Clinical Results Study—a Prospective, Phase 3, Open-Label, International Multicenter Study on Efficacy and Safety of Prophylaxis with RVWF in Severe Von Willebrand Disease

This example provides data from a study that was conducted at 32 sites in the United States (US), Canada, France, Germany, Italy, Netherlands, Russia, Spain, and Turkey. See also Tables 77-79 below.

### Pre-Assignment Details:

A total of 23 participants were enrolled and grouped into 2 cohorts: Prior On-demand and Switch (based on the previous von Willebrand disease [VWD] treatment they received prior to their enrolment in the current study) and received prophylactic treatment with recombinant von Willebrand factor (rVWF).

Prior on-Demand Participants:

Participants who had taken only on-demand von Willebrand factor (VWF) prior to the current study received rVWF (vonicog alpha) initial prophylactic treatment at a dose range of 50+/−10 international unit per kilogram (IU/kg), intravenous infusion, twice per week for up to 12 months. Dose escalation to higher dose (up to 80 IU/kg per infusion) or frequency (up to 3 times a week) was based on medical indication and investigator judgment. During this prophylaxis treatment period, any bleeding episodes requiring replacement therapy with VWF concentrate was treated with rVWF with or without recombinant Factor VIII (rFVIII; ADVATE); the dose was determined according to the bleeding type and severity, and was adjusted based on the participant's clinical response.

### Switch Participants:

Participants who had taken prophylactic treatment with plasma derived VWF product (pdVWF) prior to current study and switched to rVWF (vonicog alpha); received rVWF at an initial prophylactic dose of +/−10 percent (%) of weekly pdVWF dose received prior to this study. Dose escalation to higher dose (up to 80 IU/kg per infusion) or frequency (up to 3 times a week) was based on medical indication and investigator judgment. During this prophylaxis treatment period, any bleeding episodes requiring replacement therapy with VWF concentrate was treated with rVWF with or without recombinant Factor VIII (rFVIII; ADVATE); the dose was determined according to the bleeding type and severity, and was adjusted based on the participant's clinical response.

TABLE 11
PARTICIPANT FLOW

| | Prior On-demand | Switch | |
|---|---|---|---|
| | Participants | Participants | TOTAL |
| Started | Participants 13 | Participants 10 | 23 (calculated) |
| Treated | Participants 13 | Participants 10 | 23 (calculated) |
| Participants | | | |
| Completed | Participants 9 | Participants 8 | 17 (calculated) |
| Not Completed: | 4 (calculated) | 2 (calculated) | 6 (calculated) |
| (=Started − | | | |
| Completed) | | | |

Reason for not completed

| | | | |
|---|---|---|---|
| Total: (=sum | 4 (calculated) | 2 (calculated) | 6 (calculated) |
| per column) | | | |
| Withdrawal by | 2 | 1 | 3 (calculated) |
| Subject | | | |
| Adverse Event | 1 | 0 | 1 (calculated) |
| Other | 1 | 1 | 2 (calculated) |

TABLE 12
BASELINE CHARACTERISTICS

| | Prior On-demand | Switch | |
|---|---|---|---|
| | Participants | Participants | TOTAL |
| Overall Number of Baseline | 13 | 10 | 23 (calculated) |
| Participants | | | |
| Overall Number Of Units | | | Unknown (calculated) |
| Analyzed | | | |
| Type Of Units Analyzed: | | | |
| Age | 38.0 (17.6) | 43.9 (21.8) | 40.6 (49.3) |
| Female | 8 | 3 | 11 (calculated) |
| Male | 5 | 7 | 12 (calculated) |
| American Indian or Alaska | 0 | 0 | 0 (calculated) |
| Native | | | |
| Asian | 0 | 0 | 0 (calculated) |
| Native Hawaiian or Other | 0 | 0 | 0 (calculated) |
| Pacific Islander | | | |
| Black or African American | 0 | 0 | 0 (calculated) |
| White | 13 | 9 | 22 (calculated) |
| More than one race | 0 | 0 | 0 (calculated) |
| Unknown or Not Reported | 0 | 1 | 1 (calculated) |
| Hispanic or Latino | 0 | 2 | 2 (calculated) |
| Not Hispanic or Latino | 13 | 7 | 20 (calculated) |

| | |
|---|---|
| Baseline Analysis | Safety analysis set (SAS) composed of all |
| Population Description | participants who received any amount of |
| | investigational product (IP) (rVWF, vonicog alpha). |

### Baseline Analysis Population Description:

Safety analysis set (SAS) composed of all participants who received any amount of investigational product (IP) (rVWF, vonicog alpha).

The time frame was up to 12 months for the Primary (section 1 below) and for the secondary (sections 2-18 below).

1. Primary: Annualized Bleeding Rate (ABR) for Treated Spontaneous Bleeding Episodes (BEs) Assessed by Investigator

Description: ABR was defined as the number of treated spontaneous BEs during the treatment period/observation period (in years), where an observation period=date of completion/(termination−date of first dose+1)/365.2425. ABR for treated spontaneous bleeding episodes (not related to trauma) during prophylactic treatment with rVWF was reported. Full analysis set (FAS) was composed of all participants who received prophylaxis IP treatment.

TABLE 13

| | | Prior On-demand | | Switch | |
|---|---|---|---|---|---|
| | | Participants | | Participants | |
| | Number | 13 Participants | | 10 Participants | |
| | Analyzed: | | | | |
| ABR | Category | Number | 95% | Number | 95% |
| for | | | Confidence | | Confidence |
| treated | | | interval | | Interval |
| sponta- | | | | | |
| neous | | | | | |
| BEs | | | | | |

| | | |
|---|---|---|
| Number | 13 Participants | 10 Participants |
| Analyzed: | | |

| | | | |
|---|---|---|---|
| 0.555 | 0.150 to 2.048 | 0.283 | 0.021 to 3.851 |

2. Secondary: ABR Percent Reduction Success Achieved by Prior On-Demand Participants

Description: For prior on-demand participants, ABR percent reduction success was defined as at least 25% reduction of the ABR for treated spontaneous (not related to trauma) BEs during the first 12 months of rVWF (vonicog alfa) prophylaxis relative to the participant's own historical treated spontaneous ABR. ABR percent reduction for on-demand participants was calculated. Full analysis set (FAS) was composed of all participants who received prophylaxis IP treatment. This outcome measure was analyzed only for prior on-demand participants treated for spontaneous BEs.

TABLE 14

| | | Prior On-demand | |
|---|---|---|---|
| | | Participants | |
| ABR Percent Reduction | Category | Number | 95% |
| Success Achieved by Prior | | (Percent) | Confidence |
| On-demand Participants | | | Interval |
| Units: percent of ABR | | | |
| reduction | | | |

| | |
|---|---|
| Number | 13 Participants |
| Analyzed: | |

| | |
|---|---|
| 92.3 | 64.0 to 99.8 |

3. Secondary: ABR Preservation Success for pdVWF in Switch Participants

Description: For switch participants, ABR preservation success was defined as achieving an ABR for treated spontaneous BEs during first 12 months of rVWF (vonicog alfa) prophylaxis that was no greater than the participant's own historical ABR for treated spontaneous BEs during prophylactic treatment with pdVWF. ABR preservation success for pdVWF in switch participants was calculated. FAS composed of all participants who received prophylaxis IP treatment. This outcome measure was analyzed only for switch participants treated for spontaneous BEs.

TABLE 15

Switch Participants

| | | | |
|---|---|---|---|
| ABR Preservation | Category | Number | 95% |
| Success for pdVWF in | | (Percent) | Confidence |
| Switch Participants | | | Interval |
| Units: percent of ABR | | | |
| preservation | | | |

| | |
|---|---|
| Number | 10 Participants |
| Analyzed: | |

| | |
|---|---|
| 90.0 | 55.5 to 99.7 |

4. Secondary: Number of Participants Categorized Based on Spontaneous ABR (sABR)

Description: sABR was categorized based on number of BEs as 0, greater than (>) 0 through 2, >2 through 5, >5 during the prophylactic treatment with rVWF (vonicog alfa) through 12 months. Bleeding occurring at multiple locations related to the same injury was counted as a single bleeding episode. Bleeding episodes of unknown cause were counted as spontaneous bleeds. Number of participants based on categorized sABR was calculated. FAS composed of all participants who received prophylaxis IP treatment.

TABLE 16

| | | Prior On-demand | Switch |
|---|---|---|---|
| | | Participants | Participants |
| Number of Participants | Category | Count of | Count of |
| Categorized Based on | | Participants | Participants |
| Spontaneous ABR | | | |
| (sABR) | | | |
| Units: participants | | | |
| | Number | 13 Participants | 10 Participants |
| | Analyzed: | | |
| | 0 BEs | 11 | 7 |
| | | (84.62%) | (70%) |
| | >0 | 0 | 1 |
| | through | | |
| | 2 BEs | | |
| | | (%) | (10%) |
| | >2 | 1 | 1 |
| | through | | |
| | 5 BEs | | |
| | | (7.69%) | (10%) |
| | >5 BEs | 1 | 1 |
| | | (7.69%) | (10%) |

5. Secondary: Total Number of Infusions Administered Per Participant During Prophylactic Treatment with rVWF

Description: For each participant, the total number of infusions was counted as the total number of unique infusions of rVWF which were administered between the dates of informed consent and termination from the study, inclusive, regardless of the date and time of administration. The total number of infusions administered during the study was entered in electronic case record form (eCRF), and recorded in ERT system. Total number of infusions administered per participant during prophylactic treatment With rVWF through 12 months was calculated. The FAS was composed of all participants who received prophylaxis IP treatment. The measure type was the count of participants.

TABLE 17

| | | Prior On-demand | | Switch | |
|---|---|---|---|---|---|
| | | Participants | | Participants | |
| Total Number of | Category | Mean | Standard | Mean | Standard |
| Infusions | | | Deviation | | Deviation |
| Administered | | | | | |
| Per Participant | | | | | |
| During Prophylactic | | | | | |
| Treatment With rVWF | | | | | |
| Units: Infusion | | | | | |

| | | |
|---|---|---|
| Number | 13 Participants | 10 Participants |
| Analyzed: | | |

| | | | |
|---|---|---|---|
| 65.1 | 38.4 | 87.8 | 30.2 |

6. Secondary: Average Number of Infusions Per Week Per Participant During Prophylactic Treatment with rVWF

Description: Average number of infusions per week per participant during prophylactic treatment with rVWF through 12 months was calculated. The FAS was composed of all participants who received prophylaxis IP treatment. Units of measure: infusion.

TABLE 18

| | | Prior On-demand | | Switch | |
|---|---|---|---|---|---|
| | | Participants | | Participants | |
| Average Number of | Category | Mean | Standard | Mean | Standard |
| Infusions per Week | | | Deviation | | Deviation |
| per Participant | | | | | |
| During Prophylactic | | | | | |
| Treatment With rVWF | | | | | |
| Units: Infusion | | | | | |

| | | |
|---|---|---|
| Number | 13 Participants | 10 Participants |
| Analyzed: | | |

| | | | |
|---|---|---|---|
| 1.88 | 0.7 | 1.85 | 0.4 |

7. Secondary: Total Weight Adjusted Consumption of rVWF During Prophylactic Treatment

Description: For each participant, the body weight-adjusted dose (IU/kg) was derived as the number of units of rVWF infused (IU) divided by the last available body weight (kilogram [kg]) prior to the infusion. The total dose (IU/kg) per participant was determined as the sum of all doses. Total weight adjusted consumption of rVWF (vonicog alfa) during prophylactic treatment was reported. The FAS was composed of all participants who received prophylaxis IP treatment. Units of measure IU/kg.

TABLE 19

| | | Prior On-demand | | Switch | |
|---|---|---|---|---|---|
| | | Participants | | Participants | |
| Total Weight Adjusted | Category | Mean | Standard | Mean | Standard |
| Consumption of rVWF | | | Deviation | | Deviation |
| During Prophylactic | | | | | |
| Treatment | | | | | |
| Units: IU/kg | | | | | |

| | | |
|---|---|---|
| Number | 13 Participants | 10 Participants |
| Analyzed: | | |

| | | | |
|---|---|---|---|
| 98.6 | 40.6 | 94.0 | 30.4 |

8. Secondary: ABR for Treated Spontaneous BEs by Location of Bleeding While on Prophylactic Treatment With rVWF

Description: Spontaneous ABR by location of bleeding (for example: oral and other mucosa, menorrhagia, hemarthrosis, etc.) while on prophylactic treatment with rVWF was reported. FAS composed of all subjects who received prophylaxis IP treatment. Units: spontaneous BEs per year.

TABLE 20

| | | Prior On-demand | Switch |
|---|---|---|---|
| | | Participants | Participants |
| ABR for Treated | Category | Number | Number |
| Spontaneous BEs by | | | |
| Location of Bleeding | | | |
| While on Prophylactic | | | |
| Treatment With rVWF | | | |
| Units: Spontaneous Bes | | | |
| per year | | | |
| Oral and other mucosa | Number | 13 Participants | 10 Participants |
| | Analyzed: | | |
| | | 55.6 | 77.8 |
| Menorrhagia | Number | 13 Participants | 10 Participants |
| | Analyzed: | | |
| | | 33.3 | 0 |
| Hemarthrosis | Number | 13 Participants | 10 Participants |
| | Analyzed: | | |
| | | 0 | 5.6 |
| Other | Number | 13 Participants | 10 Participants |
| | Analyzed: | | |
| | | 12.5 | 0 |
| Unknown | Number | 13 Participants | 10 Participants |
| | Analyzed: | | |
| | | 0 | 16.7 |

9. Secondary: Number of Participants Reporting One or More Treatment-Emergent Adverse Events (TEAEs) and Serious TEAEs

Description: An adverse event (AE) is defined as any untoward medical occurrence in a participant administered IP that does not necessarily have a causal relationship with the treatment. TEAEs were events which occurred on or after the date and time of administration of the first dose of study medication. TEAEs included both serious AEs and non-serious AEs. Number of participants with TEAEs and serious TEAEs were reported. The SAS was composed of all participants who received any amount of IP (rVWF, vonicog alpha). Time frame: Up to 12 months or long as long as subjects are on treatments. Units: Subjects.

TABLE 21

| | | Prior On-demand | Switch |
|---|---|---|---|
| | | Participants | Participants |
| Number of Participants | Category | Count of | Count of |
| Reporting one or More | | Participants | Participants |
| Treatment-emergent | | | |
| Adverse Events (TEAEs) | | | |
| and Serious TEAEs | | | |
| Units: Participants | | | |
| Participants With | Number | 13 Participants | 10 Participants |
| TEAEs | Analyzed: | | |
| | | 10 (76.92%) | 7 (70%) |
| Participants with | Number | 13 Participants | 10 Participants |
| serious TEAEs | Analyzed: | | |
| | | 1 (7.69%) | 2 (20%) |

10. Secondary: Number of Participants Based on Severity of TEAEs

Description: An AE is defined as any untoward medical occurrence in a participant administered IP that does not necessarily have a causal relationship with the treatment. TEAEs were events which occurred on or after the date and time of administration of the first dose of study medication. TEAEs included both serious AEs and non-serious AEs. Severity of TEAEs was determined by following definitions: Mild: No limitation of usual activities; Moderate: Some limitation of usual activities and may required therapeutic intervention; Severe: Inability to carry out usual activities with sequelae, which required therapeutic intervention. Number of participants based on severity of TEAEs were reported. The SAS was composed of all participants who received any amount of IP (rVWF, vonicog alpha). Time frame: Up to 12 months or long as long as subjects are on treatments. Units: Subjects.

TABLE 22

| | | Prior On-demand | Switch |
|---|---|---|---|
| | | Participants | Participants |
| Number of Participants | Category | Count of | Count of |
| Based on Severity of | | Participants | Participants |
| TEAEs | | | |
| Units: Participants | | | |
| Mild | Number | 13 Participants | 10 Participants |
| | Analyzed: | | |
| | | 7 (53.85%) | 4 (40%) |
| Moderate | Number | 13 Participants | 10 Participants |
| | Analyzed: | | |
| | | 1 (7.69%) | 2 (20%) |
| Severe | Number | 13 Participants | 10 Participants |
| | Analyzed: | | |
| | | 2 (15.38%) | 1 (10%) |

11. Secondary: Number of Participants with TEAEs Based Causality

Description: An AE is defined as any untoward medical occurrence in a participant administered IP that does not necessarily ye a causal relationship with the treatment. TEAEs were events which occurred on or after the date and time of administration of the first dose of study medication. TEAEs included both serious AEs and non-serious AEs. Number of participants with TEAEs based causality was reported. The SAS was composed of all participants who received any amount of IP (rVWF, vonicog alpha). Time frame: Up to 12 months or long as long as subjects are on treatments. Units: Subjects.

TABLE 23

| | | Prior On-demand | Switch |
|---|---|---|---|
| | | Participants | Participants |
| Number of Participants | Category | Count of | Count of |
| with TEAEs Based | | Participants | Participants |
| Causality | | | |
| Units: Participants | | | |
| | Number | 13 Participants | 10 Participants |
| | Analyzed: | | |
| | | 0 (%) | 0 (%) |

12. Secondary: Number of Participants with Thromboembolic Events

Description: Thromboembolism defined as formation in a blood vessel of a clot (thrombus) that breaks loose and carried by the blood stream to plug another vessel. Number of participants with thromboembolic events as TEAEs of special interest was reported. The SAS composed of all participants who received any amount of IP (rVWF, vonicog alpha). Time frame: Up to 12 months or long as long as subjects are on treatments. Units: Subjects.

TABLE 24

| | | Prior On-demand | Switch |
|---|---|---|---|
| | | Participants | Participants |
| Number of Participants | Category | Count of | Count of |
| with Thromboembolic | | Participants | Participants |
| Events | | | |
| Units: Participants | | | |
| | Number | 13 Participants | 10 Participants |
| | Analyzed: | | |
| | | 1 (7.69%) | 0 |

13. Secondary: Number of Participants with Hypersensitivity Reactions

Description: Hypersensitivity (also called hypersensitivity reaction or intolerance) defined as undesirable reactions produced by the normal immune system, including allergies and autoimmunity. Number of participants with hypersensitivity reactions as TEAEs of special interest was calculated. The SAS composed of all participants who received any amount of IP (rVWF, vonicog alpha).

TABLE 25

| | | Prior On-demand | Switch |
|---|---|---|---|
| | | Participants | Participants |
| Number of Participants | Category | Count of | Count of |
| with Hypersensitivity | | Participants | Participants |
| Reactions | | | |
| Units: Participants | | | |
| | Number | 13 Participants | 10 Participants |
| | Analyzed: | | |
| | | 0 (0%) | 1 (10%) |

14. Secondary: Number of Participants who Developed Neutralizing Antibodies to von Willebrand Factor (rVWF) and Factor VIII (FVIII)

Description: Three functional VWF assays for von Willebrand factor collagen binding (VWF:CB), von Willebrand factor: Ristocetin Cofactor (VWF:RCo) and von Willebrand factor VIII B (VWF:FVIIIB) were used to test the presence of neutralizing anti-VWF antibodies. Neutralizing antibodies to VWF:RCo, VWF:CB and VWF:FVIIIB activities was measured by assays based on the Bethesda assay established for quantitative analysis of FVIII inhibitors (Nijmegen modification of the Bethesda assay). Only confirmed neutralizing anti-VWF antibodies were considered inhibitors. Number of participants who developed neutralizing antibodies to rVWF and FVIII were assessed. The SAS was composed of all participants who received any amount of IP (rVWF, vonicog alpha). Units: Subjects.

TABLE 26

| | | Prior On-demand | Switch |
|---|---|---|---|
| | | Participants | Participants |
| Number of Participants | Category | Count of | Count of |
| who Developed | | Participants | Participants |
| Neutralizing Antibodies | | | |
| to von Willebrand | | | |
| Factor (rVWF) and | | | |
| Factor VIII (FVIII) | | | |
| Units: Participants | | | |
| | Number | 13 Participants | 10 Participants |
| | Analyzed: | | |
| | | 0 (%) | 0 (%) |

15. Secondary: Number of Participants Who Developed of Total Binding Antibodies to Von Willebrand Factor (rVWF) and Factor VIII (FVIII)

Description: The presence of total binding anti-VWF antibodies was determined by an enzyme-linked immunosorbent assay (ELISA) employing polyclonal anti-human Immunoglobulin (Ig) antibodies (IgG, IgM and IgA). Binding antibodies against FVIII was analyzed using a proprietary enzyme immunoassay. Number of participants who developed of total binding antibodies to rVWF and FVIII was assessed. The SAS was composed of all participants who received any amount of IP (rVWF, vonicog alpha).

TABLE 27

| | | Prior On-demand | Switch |
|---|---|---|---|
| | | Participants | Participants |
| Number of Participants | Category | Count of | Count of |
| who Developed of Total | | Participants | Participants |
| Binding Antibodies to | | | |
| von Willebrand factor | | | |
| (rVWF) and Factor VIII | | | |
| (FVIII) | | | |
| Units: Participants | | | |
| | Number | 13 Participants | 10 Participants |
| | Analyzed: | | |
| | | 0 (%) | 0 (%) |

16. Secondary: Number of Participants Who Developed Binding Antibodies to Chinese Hamster Ovary (CHO) Proteins, Mouse Immunoglobulin G (IgG) and/or rFurin

Description: Total Ig antibodies (IgG, IgA, IgM) against CHO protein and human furin was analyzed using ELISA. For detection and quantification of IgG antibodies originating from human plasma that were directed against mouse-IgG (HAMA: human anti-mouse antibodies) was assessed using ELISA (Medac, Hamburg, Germany). Number of participants who developed binding antibodies to CHO proteins, Mouse IgG and/or rFurin was assessed. The SAS was composed of all participants who received any amount of IP (rVWF, vonicog alpha).

TABLE 28

| | | Prior On-demand | Switch |
|---|---|---|---|
| | | Participants | Participants |
| Number of Participants | Category | Count of | Count of |
| who Developed Binding | | Participants | Participants |
| Antibodies to Chinese | | | |
| Hamster Ovary (CHO) | | | |
| proteins, Mouse | | | |
| Immunoglobulin G | | | |
| (IgG) and/or rFurin | | | |
| Units: Participants | | | |
| | Number | 13 Participants | 10 Participants |
| | Analyzed: | | |
| | | 0 (%) | 0 (%) |

17. Secondary: Number of Participants with Clinically Significant Change from Baseline in Vital Signs

Description: Vital signs included blood pressure (systolic and diastolic), pulse rate, respiratory rate and body temperature. Number of participants with clinically significant change from baseline in vital signs was assessed. The SAS was composed of all participants who received any amount of IP (rVWF, vonicog alpha).

TABLE 29

| | | Prior On-demand | Switch |
|---|---|---|---|
| | | Participants | Participants |
| Number of Participants | Category | Count of | Count of |
| With Clinically | | Participants | Participants |
| Significant Change From | | | |
| Baseline in Vital Signs | | | |
| Units: Participants | | | |
| | Number | 13 Participants | 10 Participants |
| | Analyzed: | | |
| | | 0 (%) | 0 (%) |

18. Secondary: Number of Participants with Clinically Significant Change from Baseline in Clinical Laboratory Parameters

Description: Clinical laboratory parameters included hematology and clinical chemistry assessments. Number of participants with clinically significant change from baseline in clinical laboratory parameters was assessed. The SAS was composed of all participants who received any amount of IP (rVWF, vonicog alpha).

TABLE 30

| | | Prior On-demand | Switch |
|---|---|---|---|
| | | Participants | Participants |
| Number of Participants | Category | Count of | Count of |
| with Clinically | | Participants | Participants |
| Significant Change From | | | |
| Baseline in Clinical | | | |
| Laboratory Parameters | | | |
| Units: Participants | | | |
| | Number | 13 Participants | 10 Participants |
| | Analyzed: | | |
| | | 0 (%) | 0 (%) |

For the Secondary Sections 19-65 Below: Information on Time Frame and Analysis

The time frame at baseline was within 30 minutes pre-infusion; and post-infusion at 15, 30 minutes and 1, 3, 6, 12, 24, 30, 48, 72, and 96 hours for the secondary (sections 19-65 below).

PK full analysis set (PKFAS) composed of all participants who received at least one IP infusion and who provided at least one quantifiable pharmacokinetic or pharmacodynamic post dose measurement for pharmacokinetic and/or pharmacodynamics analysis. Here “overall number of participants analyzed” were participants who were evaluable for this outcome measure. As planned, this outcome measure was assessed only for prior on-demand participants.

19. Secondary: Pharmacokinetic (PK) Assessment: Incremental Recovery (IR) Based on Von Willebrand Factor: Ristocetin Cofactor (VWF:Rco) Activity

Description: IR at the maximum plasma concentration of VWF:RCo activity at initial PK assessment was reported. Measure type: arithmetic mean. Precision/dispersion type: standard deviation. Unit of measure: International Units per deciliter/International Units per kilogram ([IU/dL]/[IU/kg]).

TABLE 31

| | | Prior On-demand | |
|---|---|---|---|
| | | Participants | |
| Pharmacokinetic (PK) Assessment: | Category | Mean | Standard |
| Incremental Recovery (IR) Based | | | Deviation |
| on Von Willebrand Factor: | | | |
| Ristocetin Cofactor (VWF:Rco) | | | |
| Activity | | | |
| Units: (IU/dL)/(IU/kg) | | | |

| | |
|---|---|
| Number | 11 Participants |
| Analyzed: | |

| | |
|---|---|
| 1.463 | 0.3205 |

20. Secondary: Pharmacokinetic Assessment: Incremental Recovery (IR) Based on Von Willebrand Factor Antigen (VWF:Ag) Activity

Description: IR based on VWF:Ag activity at initial PK assessment was reported. Measure type: arithmetic mean. Precision/dispersion type: standard deviation. Units: ([IU/dL]/[IU/kg]).

TABLE 32

| | | Prior On-demand | |
|---|---|---|---|
| | | Participants | |
| Pharmacokinetic Assessment: | Category | Mean | Standard |
| Incremental Recovery (IR) Based | | | Deviation |
| on Von Willebrand Factor Antigen | | | |
| (VWF:Ag) Activity | | | |
| Units (IU/dL)/(IU/kg) | | | |

| | |
|---|---|
| Number | 12 Participants |
| Analyzed: | |

| | |
|---|---|
| 1.699 | 0.3488 |

21. Secondary: Pharmacokinetic Assessment: Incremental Recovery (IR) Based on Von Willebrand Factor Collagen Binding (VWF:CB) Activity

Description: IR at the maximum plasma concentration of VWF:CB activity at initial PK assessment was reported. Measure type: arithmetic mean. Precision/dispersion type: standard deviation. Units: ([IU/dL]/[IU/kg]).

TABLE 33

| | | Prior On-demand | |
|---|---|---|---|
| | | Participants | |
| Pharmacokinetic Assessment: | Category | Mean | Standard |
| Incremental Recovery (IR) Based | | | Deviation |
| on Von Willebrand Factor Collagen | | | |
| Binding (VWF:CB) Activity | | | |
| Units: (IU/dL)/(IU/kg) | | | |

| | |
|---|---|
| Number | 12 Participants |
| Analyzed: | |

| | |
|---|---|
| 2.405 | 0.5737 |

22. Secondary: Pharmacokinetic Assessment: Terminal Half-Life (T½) Based on Von Willebrand Factor: Ristocetin Cofactor (VWF:Rco) Activity

Description: T½ defined as the time in hours required for the concentration of the drug to reach half of its original value. T½ based on VWF:Rco activity at initial PK assessment was reported. Measure type: median. Precision/dispersion type: full range (min-max).

TABLE 34

| | | Prior On-demand | |
|---|---|---|---|
| | | Participants | |
| Pharmacokinetic Assessment: | Category | Median | Full Range |
| Terminal Half-life (T½) Based on | | | (min-max) |
| Von Willebrand Factor: Ristocetin | | | |
| Cofactor (VWF:Rco) Activity | | | |
| Units: hours | | | |

| | |
|---|---|
| Number | 11 Participants |
| Analyzed: | |

| | |
|---|---|
| 15.98 | 9.01 to 45.8 |

23. Secondary: Pharmacokinetic Assessment: Terminal Half-Life (T½) Based on Von Willebrand Factor Antigen (VWF:Ag) Activity

Description: T½ defined as the time in hours required for the concentration of the drug to reach half of its original value. T½ based on VWF:Ag activity at initial PK assessment was reported. Measure type: median. Precision/dispersion type: full range (min-max).

TABLE 35

| | | Prior On-demand | |
|---|---|---|---|
| | | Participants | |
| Pharmacokinetic Assessment: | Category | Median | Full Range |
| Terminal Half-life (T½) Based on | | | (min-max) |
| Von Willebrand Factor Antigen | | | |
| (VWF:Ag) Activity | | | |
| Units: hours | | | |

| | |
|---|---|
| Number | 12 Participants |
| Analyzed: | |

| | |
|---|---|
| 21.81 | 12.6 to 39.3 |

24. Secondary: Pharmacokinetic Assessment: Terminal Half-Life (T½) Based on Von Willebrand Factor Collagen Binding (VWF:CB) Activity

Description: T½ defined as the time in hours required for the concentration of the drug to reach half of its original value. T½ based on VWF:CB activity at initial PK assessment was reported. Measure type: arithmetic mean. Precision/dispersion type: standard deviation.

TABLE 36

| | | Prior On-demand | |
|---|---|---|---|
| | | Participants | |
| Pharmacokinetic Assessment: | Category | Median | Full Range |
| Terminal Half-life (T½) Based on | | | (min-max) |
| Von Willebrand Factor Collagen | | | |
| Binding (VWF:CB) Activity | | | |
| Units: hours | | | |

| | |
|---|---|
| Number | 12 Participants |
| Analyzed: | |

| | |
|---|---|
| 18.64 | 12.8 to 29.4 |

25. Secondary: Pharmacokinetic Assessment: Mean Residence Time (MRT) Based on Von Willebrand Factor: Ristocetin Cofactor (VWF:Rco) Activity

Description: MRT was calculated as (AUMC0-∞/AUC0-∞)−T½ where T1 represented the time duration of infusion, where AUMC represented the area under the first moment curve. MRT based on VWF:Rco activity at initial PK assessment was reported. Measure type: arithmetic mean. Precision/dispersion type: standard deviation.

TABLE 37

| | | Prior On-demand | |
|---|---|---|---|
| | | Participants | |
| Pharmacokinetic Assessment: | Category | Mean | Standard |
| Mean Residence Time (MRT) Based | | | Deviation |
| on Von Willebrand Factor: Ristocetin | | | |
| Cofactor (VWF:Rco) Activity | | | |
| Units: hours | | | |

| | |
|---|---|
| Number | 11 Participants |
| Analyzed: | |

| | |
|---|---|
| 23.27 | 11.00 |

26. Secondary: Pharmacokinetic Assessment: Mean Residence Time (MRT) Based on Von Willebrand Factor Antigen (VWF:Ag) Activity

Description: MRT was calculated as (AUMC0-∞/AUC0-∞)−T½ where T1 represented the time duration of infusion, where AUMC represented the area under the first moment curve. MRT based on VWF:Ag activity at initial PK assessment was reported. Measure type: arithmetic mean. Precision/dispersion type: standard deviation.

TABLE 38

| | | Prior On-demand | |
|---|---|---|---|
| | | Participants | |
| Pharmacokinetic Assessment: | Category | Mean | Standard |
| Mean Residence Time (MRT) Based | | | Deviation |
| on Von Willebrand Factor Antigen | | | |
| (VWF:Ag) Activity | | | |
| Units: hours | | | |

| | |
|---|---|
| Number | 12 Participants |
| Analyzed: | |

| | |
|---|---|
| 33.55 | 9.777 |

27. Secondary: Pharmacokinetic Assessment: Mean Residence Time (MRT) Based on Von Willebrand Factor Collagen Binding (VWF:CB) Activity

Description: MRT was calculated as (AUMC0-∞/AUC0-∞)−T½ where T1 represented the time duration of infusion, where AUMC represented the area under the first moment curve. MRT based on VWF:CB activity at initial PK assessment was reported. Measure type: arithmetic mean. Precision/dispersion type: standard deviation.

TABLE 39

| | | Prior On-demand | |
|---|---|---|---|
| | | Participants | |
| Pharmacokinetic Assessment: | Category | Mean | Standard |
| Mean Residence Time (MRT) Based | | | Deviation |
| on Von Willebrand Factor Collagen | | | |
| Binding (VWF:CB) Activity | | | |
| Units: hours | | | |

| | |
|---|---|
| Number | 12 Participants |
| Analyzed: | |

| | |
|---|---|
| 27.39 | 6.860 |

28. Secondary: Pharmacokinetic Assessment: Area Under the Plasma Concentration Versus Time Curve from Time 0 Extrapolated to Infinity (AUC0-∞) Based on Von Willebrand Factor: Ristocetin Cofactor (VWF:Rco) Activity

Description: AUC0-∞ based on VWF:Rco activity at initial PK assessment was reported. Measure type: arithmetic mean. Precision/dispersion type: standard deviation. Unit of measure: International Units*hour per deciliter (IU*h/dL).

TABLE 40

| | | Prior On-demand | |
|---|---|---|---|
| | | Participants | |
| Pharmacokinetic Assessment: | Category | Mean | Standard |
| Area Under the Plasma Concentration | | | Deviation |
| Versus Time Curve From Time 0 | | | |
| Extrapolated to Infinity (AUC0-∞) | | | |
| Based on Von Willebrand Factor: | | | |
| Ristocetin Cofactor (VWF:Rco) Activity | | | |
| UnitsL IU*h/dL | | | |

| | |
|---|---|
| Number | 11 Participants |
| Analyzed: | |

| | |
|---|---|
| 1199 | 467.8 |

29. Secondary: Pharmacokinetic Assessment: Area Under the Plasma Concentration Versus Time Curve from Time 0 Extrapolated to Infinity (AUC0-∞) Based on Von Willebrand Factor Antigen (VWF:Ag) Activity

Description: AUC0-∞ based on VWF:Ag activity at initial PK assessment was reported. Measure type: arithmetic mean. Precision/dispersion type: standard deviation.

TABLE 41

| | | Prior On-demand | |
|---|---|---|---|
| | | Participants | |
| Pharmacokinetic Assessment: | Category | Mean | Standard |
| Area Under the Plasma Concentration | | | Deviation |
| Versus Time Curve From Time 0 | | | |
| Extrapolated to Infinity (AUC0-∞) | | | |
| Based on Von Willebrand Factor | | | |
| Antigen (VWF:Ag) Activity | | | |
| Units: IU*h/dL | | | |

| | |
|---|---|
| Number | 12 Participants |
| Analyzed: | |

| | |
|---|---|
| 2578 | 1067 |

30. Secondary: Pharmacokinetic Assessment: Area Under the Plasma Concentration Versus Time Curve from Time 0 Extrapolated to Infinity (AUC0-∞) Based on Von Willebrand Factor Collagen Binding (VWF:CB) Activity

Description: AUC0-∞ based on VWF:CB activity at initial PK assessment was reported. Measure type: arithmetic mean. Precision/dispersion type: standard deviation.

TABLE 42

| | | Prior On-demand | |
|---|---|---|---|
| | | Participants | |
| Pharmacokinetic Assessment: | Category | Mean | Standard |
| Area Under the Plasma Concentration | | | Deviation |
| Versus Time Curve From Time 0 | | | |
| Extrapolated to Infinity (AUC0-∞) | | | |
| Based on Von Willebrand Factor | | | |
| Collagen Binding (VWF:CB) Activity | | | |
| Units: IU*h/dL | | | |

| | |
|---|---|
| Number | 12 Participants |
| Analyzed: | |

| | |
|---|---|
| 3010 | 1221 |

31. Secondary: Pharmacokinetic Assessment: Area Under the Plasma Concentration Versus Time Curve from Time 0 to the Last Measurable Concentration (AUC0-tlast) Based on Von Willebrand Factor: Ristocetin Cofactor (VWF:Rco) Activity

Description: AUC0-tlast based on VWF:Rco activity at initial PK assessment was reported. Measure type: arithmetic mean. Precision/dispersion type: standard deviation.

TABLE 43

| | | Prior On-demand | |
|---|---|---|---|
| | | Participants | |
| Pharmacokinetic Assessment: | Category | Mean | Standard |
| Area Under the Plasma Concentration | | | Deviation |
| Versus Time Curve From Time 0 to the | | | |
| Last Measurable Concentration | | | |
| (AUC0-tlast) Based on Von Willebrand | | | |
| Factor: Ristocetin Cofactor (VWF:Rco) | | | |
| Activity | | | |
| Units: IU*h/dL | | | |

| | |
|---|---|
| Number | 12 Participants |
| Analyzed: | |

| | |
|---|---|
| 919.8 | 378.4 |

32. Secondary: Pharmacokinetic Assessment: Area Under the Plasma Concentration Versus Time Curve from Time 0 to the Last Measurable Concentration (AUC0-tlast) Based on Von Willebrand Factor Antigen (VWF:Ag) Activity

Description: AUC0-tlast based on VWF:Ag activity at initial PK assessment was reported. Measure type: arithmetic mean. Precision/dispersion type: standard deviation.

TABLE 44

| | | Prior On-demand | |
|---|---|---|---|
| | | Participants | |
| Pharmacokinetic Assessment: | Category | Mean | Standard |
| Area Under the Plasma | | | Deviation |
| Concentration Versus Time | | | |
| Curve From Time 0 to the | | | |
| Last Measurable Concentration | | | |
| (AUC0-tlast) Based on Von | | | |
| Willebrand Factor Antigen | | | |
| (VWF:Ag) Activity | | | |
| Units: IU*h/dL | | | |

| | |
|---|---|
| Number | 12 Participants |
| Analyzed: | |

| | |
|---|---|
| 2357 | 848.6 |

33. Secondary: Pharmacokinetic Assessment: Area Under the Plasma Concentration Versus Time Curve From Time 0 to the Last Measurable Concentration (AUC0-tlast) Based on Von Willebrand Factor Collagen Binding (VWF:CB) Activity

Description: AUC0-tlast based on VWF:CB activity at initial PK assessment was reported. Measure type: arithmetic mean. Precision/dispersion type: standard deviation.

TABLE 45

| | | Prior On-demand | |
|---|---|---|---|
| | | Participants | |
| Pharmacokinetic Assessment: | Category | Mean | Standard |
| Area Under the Plasma Concentration | | | Deviation |
| Versus Time Curve From Time 0 to the | | | |
| Last Measurable Concentration | | | |
| (AUC0-tlast) Based on Von Willebrand | | | |
| Factor Collagen Binding (VWF:CB) | | | |
| Activity | | | |
| Units: IU*h/dL | | | |

| | |
|---|---|
| Number | 12 Participants |
| Analyzed: | |

| | |
|---|---|
| 2824 | 1112 |

34. Secondary: Pharmacokinetic Assessment: Maximum Plasma Concentration (Cmax) Based on Von Willebrand Factor: Ristocetin Cofactor (VWF:Rco) Activity

Description: Cmax based on VWF:Rco at initial PK assessment was reported. Measure type: arithmetic mean. Precision/dispersion type: standard deviation. Unit of measure: International Units per deciliter.

TABLE 46

| | | Prior On-demand | |
|---|---|---|---|
| | | Participants | |
| Pharmacokinetic Assessment: | Category | Mean | Standard |
| Maximum Plasma Concentration | | | Deviation |
| (Cmax) Based on Von Willebrand | | | |
| Factor: Ristocetin Cofactor | | | |
| (VWF:Rco) Activity | | | |
| Units: IU/dL | | | |

| | |
|---|---|
| Number | 12 Participants |
| Analyzed: | |

| | |
|---|---|
| 74.62 | 16.09 |

35. Secondary: Pharmacokinetic Assessment: Maximum Plasma Concentration (Cmax) Based on Von Willebrand Factor Antigen (VWF:Ag) Activity

Description: Cmax based on VWF:Ag activity at initial PK assessment was reported. Measure type: arithmetic mean. Precision/dispersion type: standard deviation.

TABLE 47

| | | Prior On-demand | |
|---|---|---|---|
| | | Participants | |
| Pharmacokinetic Assessment: | Category | Mean | Standard |
| Maximum Plasma Concentration | | | Deviation |
| (Cmax) Based on Von Willebrand | | | |
| Factor Antigen (VWF:Ag) Activity | | | |
| Units: IU/dL | | | |

| | |
|---|---|
| Number | 12 Participants |
| Analyzed: | |

| | |
|---|---|
| 85.1 | 19.2 |

36. Secondary: Pharmacokinetic Assessment: Maximum Plasma Concentration (Cmax) Based on Von Willebrand Factor Collagen Binding (VWF:CB) Activity.

Description: Cmax based on VWF:CB activity at initial PK assessment was reported. Measure type: arithmetic mean. Precision/dispersion type: standard deviation.

TABLE 48

| | | Prior On-demand | |
|---|---|---|---|
| | | Participants | |
| Pharmacokinetic Assessment: | Category | Mean | Standard |
| Maximum Plasma Concentration | | | Deviation |
| (Cmax) Based on Von Willebrand | | | |
| Factor Collagen Binding | | | |
| (VWF:CB) Activity | | | |
| Units: IU/dL | | | |

| | |
|---|---|
| Number | 12 Participants |
| Analyzed: | |

| | |
|---|---|
| 120.74 | 29.83 |

37. Secondary: Pharmacokinetic Assessment: Minimum Time to Reach the Maximum Plasma Concentration (Tmax) Based on Von Willebrand Factor: Ristocetin Cofactor (VWF:Rco) Activity.

Description: Tmax based on VWF:Rco activity at initial PK assessment was reported. Measure type: median. Precision/dispersion type: Full range (min-max).

TABLE 49

| | | Prior On-demand | |
|---|---|---|---|
| | | Participants | |
| Pharmacokinetic Assessment: | Category | Median | Full Range |
| Minimum Time to Reach the | | | |
| Maximum Plasma Concentration | | | |
| (Tmax) Based on Von Willebrand | | | |
| Factor: Ristocetin Cofactor | | | |
| (VWF:Rco) Activity. | | | |
| Units: hours | | | |

| | |
|---|---|
| Number | 12 Participants |
| Analyzed: | |

| | |
|---|---|
| 0.540 | 0.27 to 1.02 |

38. Secondary: Pharmacokinetic Assessment: Minimum Time to Reach the Maximum Plasma Concentration (Tmax) Based on Von Willebrand Factor Antigen (VWF:Ag) Activity

Description: Tmax based on VWF:Ag activity at initial PK assessment was reported. Measure type: median. Precision/dispersion type: Full range (min-max).

TABLE 50

| | | Prior On-demand | |
|---|---|---|---|
| | | Participants | |
| Pharmacokinetic Assessment: | Category | Median | Full Range |
| Minimum Time to Reach the | | | |
| Maximum Plasma Concentration | | | |
| (Tmax) Based on Von Willebrand | | | |
| Factor Antigen (VWF:Ag) Activity | | | |
| Units: hours | | | |

| | |
|---|---|
| Number | 12 Participants |
| Analyzed: | |

| | |
|---|---|
| 1 | 0.27 to 3.25 |

39. Secondary: Pharmacokinetic Assessment: Minimum Time to Reach the Maximum Plasma Concentration (Tmax) Based on Von Willebrand Factor Collagen Binding (VWF:CB) Activity.

Description: Tmax based on VWF:CB activity at initial PK assessment was reported. Measure type: median. Precision/dispersion type: Full range (min-max).

TABLE 51

| | | Prior On-demand | |
|---|---|---|---|
| | | Participants | |
| Pharmacokinetic Assessment: | Category | Median | Full Range |
| Minimum Time to Reach the | | | |
| Maximum Plasma Concentration | | | |
| (Tmax) Based on Von Willebrand | | | |
| Factor Antigen (VWF:Ag) Activity | | | |
| Units: hours | | | |

| | |
|---|---|
| Number | 12 Participants |
| Analyzed: | |

| | |
|---|---|
| 0.500 | 0.27 to 1.02 |

40. Secondary: Pharmacokinetic Assessment: Volume of Distribution at Steady State (Vss) Based on Von Willebrand Factor: Ristocetin Cofactor (VWF:Rco) Activity.

Description: Volume of distribution is defined as the theoretical volume in which the total amount of drug would need to be uniformly distributed to produce the desired plasma concentration of a drug. Vss based on VWF:Rco activity at initial assessment was reported. Measure type: arithmetic mean. Precision/dispersion type: standard deviation.

TABLE 52

| | | Prior On-demand | |
|---|---|---|---|
| | | Participants | |
| Pharmacokinetic Assessment: | Category | Mean | Standard |
| Volume of Distribution at | | | Deviation |
| Steady State (Vss) Based on | | | |
| Von Willebrand Factor: Ristocetin | | | |
| Cofactor (VWF:Rco) Activity. | | | |
| Units: dL/kg | | | |

| | |
|---|---|
| Number | 11 Participants |
| Analyzed: | |

| | |
|---|---|
| 1.052 | 0.4981 |

41. Secondary: Pharmacokinetic Assessment: Volume of Distribution at Steady State (Vss) Based on Von Willebrand Factor Antigen (VWF:Ag) Activity.

Volume of distribution is defined as the theoretical volume in which the total amount of drug would need to be uniformly distributed to produce the desired plasma concentration of a drug. Vss based on VWF:Ag activity at initial assessment was reported. Measure type: arithmetic mean. Precision/dispersion type: standard deviation.

TABLE 53

| | | Prior On-demand | |
|---|---|---|---|
| | | Participants | |
| Pharmacokinetic Assessment: | Category | Mean | Standard |
| Volume of Distribution at | | | Deviation |
| Steady State (Vss) Based on | | | |
| Von Willebrand Factor Antigen | | | |
| (VWF:Ag) Activity | | | |
| Units: dL/kg | | | |

| | |
|---|---|
| Number | 12 Participants |
| Analyzed: | |

| | |
|---|---|
| 0.6860 | 0.1556 |

42. Secondary: Pharmacokinetic Assessment: Volume of Distribution at Steady State (Vss) Based on Von Willebrand Factor Collagen Binding (VWF:CB) Activity

Description: Volume of distribution is defined as the theoretical volume in which the total amount of drug would need to be uniformly distributed to produce the desired plasma concentration of a drug. Vss based on VWF:CB activity at initial assessment was reported. Measure type: arithmetic mean. Precision/dispersion type: standard deviation.

TABLE 54

| | | Prior On-demand | |
|---|---|---|---|
| | | Participants | |
| Pharmacokinetic Assessment: | Category | Mean | Standard |
| Volume of Distribution at | | | Deviation |
| Steady State (Vss) Based on | | | |
| Von Willebrand Factor Collagen | | | |
| Binding (VWF:CB) Activity | | | |
| Units: dL/kg | | | |

| | |
|---|---|
| Number | 12 Participants |
| Analyzed: | |

| | |
|---|---|
| 0.4889 | 0.1371 |

43. Secondary: Pharmacokinetic Assessment: Clearance (CL) Based on Von Willebrand Factor: Ristocetin Cofactor (VWF:Rco) Activity

Description: Clearance is defined as a quantitative measure of the rate at which a drug substance is removed from the body. CL based on VWF:Rco activity at initial PK assessment was reported. Measure type: arithmetic mean. Precision/dispersion type: standard deviation. Unit of measure: deciliter per kilogram per hour (dL/kg/h).

TABLE 55

| | | Prior On-demand | |
|---|---|---|---|
| | | Participants | |
| Pharmacokinetic Assessment: | Category | Mean | Standard |
| Clearance (CL) Based on Von | | | Deviation |
| Willebrand Factor: Ristocetin | | | |
| Cofactor (VWF:Rco) Activity | | | |
| Units: dL/kg/h | | | |

| | |
|---|---|
| Number | 11 Participants |
| Analyzed: | |

| | |
|---|---|
| 0.04765 | 0.01620 |

44. Secondary: Pharmacokinetic Assessment: Clearance (CL) Based on Von Willebrand Factor Antigen (VWF:Ag) Activity

Description: Clearance is defined as a quantitative measure of the rate at which a drug substance is removed from the body. CL based on VWF:Ag activity at initial PK assessment was reported. Measure type: arithmetic mean. Precision/dispersion type: standard deviation.

TABLE 56

| | | Prior On-demand | |
|---|---|---|---|
| | | Participants | |
| Pharmacokinetic Assessment: | Category | Mean | Standard |
| Clearance (CL) Based on Von | | | Deviation |
| Willebrand Factor Antigen | | | |
| (VWF:Ag) Activity | | | |
| Units: dL/kg/h | | | |

| | |
|---|---|
| Number | 12 Participants |
| Analyzed: | |

| | |
|---|---|
| 0.02185 | 0.006821 |

45. Secondary: Pharmacokinetic Assessment: Clearance (CL) Based on Von Willebrand Factor Collagen Binding (VWF:CB) Activity

Description: Clearance is defined as a quantitative measure of the rate at which a drug substance is removed from the body. CL based on VWF:CB activity at initial PK assessment was reported. Measure type: arithmetic mean. Precision/dispersion type: standard deviation.

TABLE 57

| | | Prior On-demand | |
|---|---|---|---|
| | | Participants | |
| Pharmacokinetic Assessment: | Category | Mean | Standard |
| Clearance (CL) Based on Von | | | Deviation |
| Willebrand Factor Collagen | | | |
| Binding (VWF:CB) Activity | | | |
| Units: dL/kg/h | | | |

| | |
|---|---|
| Number | 12 Participants |
| Analyzed: | |

| | |
|---|---|
| 0.01872 | 0.005946 |

46. Secondary: Pharmacodynamic (PD) Assessment: Maximum Plasma Concentration (Cmax) Based on Factor VIII Clotting (FVIII:C) Activity

Description: Cmax based on FVIII:C activity at initial PD assessment by the 1-stage clotting assay was reported. Measure type: arithmetic mean. Precision/dispersion type: standard deviation.

TABLE 58

| | | Prior On-demand | |
|---|---|---|---|
| | | Participants | |
| Pharmacodynamic (PD) Assessment: | Category | Mean | Standard |
| Maximum Plasma Concentration | | | Deviation |
| (Cmax) Based on Factor VIII | | | |
| Clotting (FVIII:C) Activity | | | |
| Units: IU/dL | | | |

| | |
|---|---|
| Number | 12 Participants |
| Analyzed: | |

| | |
|---|---|
| 90.8 | 32.1 |

47. Secondary: Pharmacodynamic Assessment: Minimum Time to Reach the Maximum Plasma Concentration (Tmax) Based on Factor VIII Clotting (FVIII:C) Activity

Description: Tmax based on FVIII:C activity at initial PD assessment by the 1-stage clotting assay was reported. Measure type: arithmetic mean. Precision/dispersion type: standard deviation.

TABLE 59

| | | Prior On-demand | |
|---|---|---|---|
| | | Participants | |
| Pharmacodynamic Assessment: | Category | Median | Full Range |
| Minimum Time to Reach the | | | |
| Maximum Plasma Concentration | | | |
| (Tmax) Based on Factor VIII | | | |
| Clotting (FVIII:C) Activity | | | |
| Units: hours | | | |

| | |
|---|---|
| Number | 12 Participants |
| Analyzed: | |

| | |
|---|---|
| 24.055 | 11.98 to 46.30 |

48. Secondary: Pharmacodynamic Assessment: Area Under the Plasma Concentration Versus Time Curve From Time 0 to the Last Measurable Concentration (AUC0-tlast) Based on Factor VIII Clotting (FVIII:C) Activity

Description: AUC0-tlast based on FVIII:C activity at initial PD assessment by the 1-stage clotting assay was reported. Measure type: arithmetic mean. Precision/dispersion type: standard deviation.

TABLE 60

| | | Prior On-demand | |
|---|---|---|---|
| | | Participants | |
| Pharmacodynamic Assessment: | Category | Mean | Standard |
| Area Under the Plasma Concentration | | | Deviation |
| Versus Time Curve From Time 0 to the | | | |
| Last Measurable Concentration | | | |
| (AUC0-tlast) Based on Factor VIII | | | |
| Clotting (FVIII:C) Activity | | | |
| Units: IU*h/dL | | | |

| | |
|---|---|
| Number | 12 Participants |
| Analyzed: | |

| | |
|---|---|
| 4949 | 2436 |

49. Secondary: Pharmacodynamic Assessment at Steady State: Area Under the Plasma Concentration Versus Time Curve From Time 0 to end of the Partial Dosing Interval (AUC0-tau;ss) Based on Von Willebrand Factor: Ristocetin Cofactor (VWF:Rco) Activity

Description: AUC0-tau;ss based on VWF:Rco activity at steady state during end of study (Month 12) was reported. Measure type: arithmetic mean. Precision/dispersion type: standard deviation. Data not provided.

50. Secondary: Pharmacokinetic Assessment at Steady State: Area Under the Plasma Concentration Versus Time Curve From Time 0 to end of the Partial Dosing Interval (AUC0-tau;ss) Based on Von Willebrand Factor Collagen Binding (VWF:CB) Activity

Description: AUC0-tau;ss based on VWF:CB activity at steady state during end of study (Month 12) was reported. Measure type: arithmetic mean. Precision/dispersion type: standard deviation. Data not provided.

51. Secondary: Pharmacokinetic Assessment at Steady State: Area Under the Plasma Concentration Versus Time Curve from Time 0 to End of the Partial Dosing Interval (AUC0-tau;ss) Based on Von Willebrand Factor Collagen Binding (VWF:CB) Activity

Description: AUC0-tau;ss based on VWF:CB activity at steady state during end of study (Month 12) was reported. Measure type: arithmetic mean. Precision/dispersion type: standard deviation. Data not provided.

52. Secondary: Pharmacokinetics Assessment at Steady State: Maximum Plasma Concentration During the Partial Dosing Interval at Steady State (Cmax;ss) Based on Von Willebrand Factor: Ristocetin Cofactor (VWF:Rco) Activity

Description: Cmax;ss based on VWF:Rco activity at steady state during end of study (Month 12) was reported. Measure type: arithmetic mean. Precision/dispersion type: standard deviation.

TABLE 61

| | | Prior On-demand | | Switch | |
|---|---|---|---|---|---|
| | | Participants | | Participants | |
| Pharmacokinetics Assessment | Category | Mean | Standard | Mean | Standard |
| at Steady State: Maximum Plasma | | | Deviation | | Deviation |
| Concentration During the Partial | | | | | |
| Dosing Interval at Steady State | | | | | |
| (Cmax; ss) Based on Von | | | | | |
| Willebrand Factor: Ristocetin | | | | | |
| Cofactor (VWF:Rco) Activity | | | | | |
| Units: IU/dL | | | | | |

| | | |
|---|---|---|
| Number | 9 Participants | 7 Participants |
| Analyzed: | | |

| | | | |
|---|---|---|---|
| 92.63 | 37.05 | 102.89 | 44.74 |

53. Secondary: Pharmacokinetics Assessment at Steady State: Maximum Plasma Concentration During the Partial Dosing Interval at Steady State (Cmax;ss) Based on Von Willebrand Factor Antigen (VWF:Ag) Activity

Description: Cmax;ss based on VWF:Ag activity at steady state during end of study (Month 12) was reported. Measure type: arithmetic mean. Precision/dispersion type: standard deviation.

TABLE 62

| | | Prior On-demand | | Switch | |
|---|---|---|---|---|---|
| | | Participants | | Participants | |
| Pharmacokinetics Assessment | Category | Mean | Standard | Mean | Standard |
| at Steady State: Maximum | | | Deviation | | Deviation |
| Plasma Concentration During | | | | | |
| the Partial Dosing Interval at | | | | | |
| Steady State (Cmax; ss) Based | | | | | |
| on Von Willebrand Factor | | | | | |
| Antigen (VWF:Ag) Activity | | | | | |
| Units: IU/dL | | | | | |

| | | |
|---|---|---|
| Number | 9 Participants | 7 Participants |
| Analyzed: | | |

| | | | |
|---|---|---|---|
| 108.1 | 39.6 | 107.1 | 37.4 |

54. Secondary: Pharmacokinetics Assessment at Steady State: Maximum Plasma Concentration During the Partial Dosing Interval at Steady State (Cmax;ss) Based on Von Willebrand Factor Collagen Binding (VWF:CB) Activity

Description: Cmax;ss based on VWF:CB activity at steady state during end of study (Month 12) was reported. Measure type: arithmetic mean. Precision/dispersion type: standard deviation.

TABLE 63

| | | Prior On-demand | | Switch | |
|---|---|---|---|---|---|
| | | Participants | | Participants | |
| Pharmacokinetics Assessment at | Category | Mean | Standard | Mean | Standard |
| Steady State: Maximum Plasma | | | Deviation | | Deviation |
| Concentration During the Partial | | | | | |
| Dosing Interval at Steady State | | | | | |
| (Cmax; ss) Based on Von | | | | | |
| Willebrand Factor Collagen | | | | | |
| Binding (VWF:CB) Activity | | | | | |
| Units: IU/dL | | | | | |

| | | |
|---|---|---|
| Number | 9 Participants | 7 Participants |
| Analyzed: | | |

| | | | |
|---|---|---|---|
| 137.48 | 44.97 | 162.19 | 60.04 |

55. Secondary: Pharmacokinetics Assessment at Steady State: Minimum Time to Reach the Maximum Plasma Concentration at Steady State (Tmax;ss) Based on Von Willebrand Factor: Ristocetin Cofactor (VWF:Rco) Activity

Description: Tmax;ss based on VWF:Rco activity at steady state during end of study (Month 12) was reported. Measure type: median. Precision/dispersion type: full range (min-max).

TABLE 64

| | | Prior On-demand | | Switch | |
|---|---|---|---|---|---|
| | | Participants | | Participants | |
| Pharmacokinetics Assessment at | Category | Median | Full Range | Median | Full Range |
| Steady State: Minimum Time | | | | | |
| to Reach the Maximum Plasma | | | | | |
| Concentration at Steady State | | | | | |
| (Tmax; ss) Based on Von | | | | | |
| Willebrand Factor: Ristocetin | | | | | |
| Cofactor (VWF:Rco) Activity | | | | | |
| Units: hours | | | | | |

| | | |
|---|---|---|
| Number | 9 Participants | 7 Participants |
| Analyzed: | | |

| | | | |
|---|---|---|---|
| 0.330 | 0.25 to 1.25 | 0.400 | 0.33 to 0.52 |

56. Secondary: Pharmacokinetics Assessment at Steady State: Minimum Time to Reach the Maximum Plasma Concentration at Steady State (Tmax;ss) Based on Von Willebrand Factor Antigen (VWF:Ag) Activity

Description: Tmax;ss based on VWF:Ag activity at steady state during end of study (Month 12) was reported. Measure type: median. Precision/dispersion type: full range (min-max).

TABLE 65

| | | Prior On-demand | | Switch | |
|---|---|---|---|---|---|
| | | Participants | | Participants | |
| Pharmacokinetics Assessment | Category | Median | Full Range | Median | Full Range |
| at Steady State: Minimum | | | | | |
| Time to Reach the Maximum | | | | | |
| Plasma Concentration at | | | | | |
| Steady State (Tmax; ss) Based | | | | | |
| on Von Willebrand Factor | | | | | |
| Antigen (VWF:Ag) Activity | | | | | |
| Units: hours | | | | | |

| | | |
|---|---|---|
| Number | 9 Participants | 7 Participants |
| Analyzed: | | |

| | | | |
|---|---|---|---|
| 0.580 | 0.28 to 1.17 | 0.330 | 0.23 to 1.17 |

57. Secondary: Pharmacokinetics Assessment at Steady State: Minimum Time to Reach the Maximum Plasma Concentration at Steady State (Tmax;ss) Based on Von Willebrand Factor Collagen Binding (VWF:CB) Activity

Description: Tmax;ss based on VWF:CB activity at steady state during end of study (Month 12) was reported. Measure type: median. Precision/dispersion type: full range (min-max).

TABLE 66

| | | Prior On-demand | | Switch | |
|---|---|---|---|---|---|
| | | Participants | | Participants | |
| Pharmacokinetics Assessment at | Category | Median | Full Range | Median | Full Range |
| Steady State: Minimum Time | | | | | |
| to Reach the Maximum Plasma | | | | | |
| Concentration at Steady State | | | | | |
| (Tmax; ss) Based on Von | | | | | |
| Willebrand Factor Collagen | | | | | |
| Binding (VWF:CB) Activity | | | | | |
| Units: hours | | | | | |

| | | |
|---|---|---|
| Number | 9 Participants | 7 Participants |
| Analyzed: | | |

| | | | |
|---|---|---|---|
| 0.420 | 0.28 to 3.00 | 0.670 | 0.33 to 3.02 |

58. Secondary: Pharmacokinetics Assessment at Steady State: Minimum Plasma Concentration During the Partial Dosing Interval at Steady State (Cmin;ss) Based on Von Willebrand Factor: Ristocetin Cofactor (VWF:Rco) Activity

Description: Cmin;ss based on VWF:Rco activity at steady state during end of study (Month 12) was reported. Measure type: arithmetic mean. Precision/dispersion type: standard deviation.

TABLE 67

| | | Prior On-demand | | Switch | |
|---|---|---|---|---|---|
| | | Participants | | Participants | |
| Pharmacokinetics Assessment at | Category | Mean | Standard | Mean | Standard |
| Steady State: Minimum Plasma | | | Deviation | | Deviation |
| Concentration During the Partial | | | | | |
| Dosing Interval at Steady State | | | | | |
| (Cmin; ss) Based on Von | | | | | |
| Willebrand Factor: Ristocetin | | | | | |
| Cofactor (VWF:Rco) Activity | | | | | |
| Units: IU/dL | | | | | |

| | | |
|---|---|---|
| Number | 9 Participants | 7 Participants |
| Analyzed: | | |

| | |
|---|---|
| NA | NA |

| | |
|---|---|
| Data not reported | Data not reported |
| as value was | as value was |
| below the limit of | below the limit of |
| quantification. | quantification. |

59. Secondary: Pharmacokinetics Assessment at Steady State: Minimum Plasma Concentration During the Partial Dosing Interval at Steady State (Cmin;ss) Based on Von Willebrand Factor Antigen (VWF:Ag) Activity

Description: Cmin;ss based on VWF:Rco activity at steady state during end of study (Month 12) was reported. Measure type: arithmetic mean. Precision/dispersion type: standard deviation.

TABLE 68

| | | Prior On-demand | | Switch | |
|---|---|---|---|---|---|
| | | Participants | | Participants | |
| Pharmacokinetics Assessment | Category | Mean | Standard | Mean | Standard |
| at Steady State: Minimum | | | Deviation | | Deviation |
| Plasma Concentration During | | | | | |
| the Partial Dosing Interval at | | | | | |
| Steady State (Cmin; ss) Based | | | | | |
| on Von Willebrand Factor | | | | | |
| Antigen (VWF:Ag) Activity | | | | | |
| Units: IU/dL | | | | | |

| | | |
|---|---|---|
| Number | 9 Participants | 7 Participants |
| Analyzed: | | |

| | | | |
|---|---|---|---|
| 6.3 | 4.8 | 11.7 | 8.9 |

60. Secondary: Pharmacokinetics Assessment at Steady State: Minimum Plasma Concentration During the Partial Dosing Interval at Steady State (Cmin;ss) Based on Von Willebrand Factor Collagen Binding (VWF:CB) Activity

Description: Cmin;ss based on VWF:CB activity at steady state during end of study (Month 12) was reported. Measure type: arithmetic mean. Precision/dispersion type: standard deviation.

TABLE 69

| | | Prior On-demand | | Switch | |
|---|---|---|---|---|---|
| | | Participants | | Participants | |
| Pharmacokinetics Assessment | Category | Mean | Standard | Mean | Standard |
| at Steady State: Minimum | | | Deviation | | Deviation |
| Plasma Concentration During | | | | | |
| the Partial Dosing Interval at | | | | | |
| Steady State (Cmin; ss) Based | | | | | |
| on Von Willebrand Factor Collagen | | | | | |
| Binding (VWF:CB) Activity | | | | | |
| Units: IU/dL | | | | | |

| | | |
|---|---|---|
| Number | 9 Participants | 7 Participants |
| Analyzed: | | |

| | | | |
|---|---|---|---|
| 3.82 | 6.86 | 9.94 | 8.46 |

61. Secondary: harmacodynamic Assessment at Steady State: Area Under the Plasma Concentration Versus Time Curve from Time 0 to End of the Partial Dosing Interval (AUC0-tau;ss) Based on Factor VIII Clotting (FVIII:C) Activity.

Description: AUC0-tau;ss based on FVIII:C activity at steady state during end of study (Month 12) was reported. Measure type: arithmetic mean. Precision/dispersion type: standard deviation. Data not provided.

62. Secondary: Pharmacodynamic Assessment at Steady State: Maximum Plasma Concentration During the Partial Dosing Interval at Steady State (Cmax;ss) Based on Factor VIII Clotting (FVIII:C) Activity

Description: Cmax;ss based on FVIII:C activity was assessed at steady state during end of study (Month 12) was reported. Measure type: arithmetic mean. Precision/dispersion type: standard deviation.

TABLE 70

| | | Prior On-demand | | Switch | |
|---|---|---|---|---|---|
| | | Participants | | Participants | |
| Pharmacodynamic Assessment | Category | Mean | Standard | Mean | Standard |
| at Steady State: Maximum | | | Deviation | | Deviation |
| Plasma Concentration During | | | | | |
| the Partial Dosing Interval at | | | | | |
| Steady State (Cmax; ss) Based | | | | | |
| on Factor VIII Clotting | | | | | |
| (FVIII:C) Activity | | | | | |
| Units: IU/dL | | | | | |

| | | |
|---|---|---|
| Number | 9 Participants | 7 Participants |
| Analyzed: | | |

| | | | |
|---|---|---|---|
| 104.1 | 35.1 | 75.7 | 37.3 |

63. Secondary: Pharmacodynamic Assessment at Steady State: Minimum Time to Reach the Maximum Plasma Concentration at Steady State (Tmax;ss) Based on Factor VIII Clotting (FVIII:C) Activity

Description: Tmax;ss based on FVIII:C activity at steady state during end of study (Month 12) was reported. Measure type: arithmetic mean. Precision/dispersion type: standard deviation.

TABLE 71

| | | Prior On-demand | | Switch | |
|---|---|---|---|---|---|
| | | Participants | | Participants | |
| Pharmacodynamic Assessment | Category | Median | Full Range | Median | Full Range |
| at Steady State: Minimum | | | | | |
| Time to Reach the Maximum | | | | | |
| Plasma Concentration at | | | | | |
| Steady State (Tmax; ss) | | | | | |
| Based on Factor VIII | | | | | |
| Clotting (FVIII:C) Activity | | | | | |
| Units: hours | | | | | |

| | | |
|---|---|---|
| Number | 9 Participants | 7 Participants |
| Analyzed: | | |

| | | | |
|---|---|---|---|
| 24.500 | 6.17 to 29.27 | 24.070 | 1.08 to 30.03 |

64. Secondary: Pharmacodynamic Assessment at Steady State: Minimum Plasma Concentration During the Partial Dosing Interval at Steady State (Cmin;ss) Based on Factor VIII Clotting (FVIII:C) Activity

Description: Cmin;ss based on FVIII:C activity at steady state during end of study (Month 12) was reported. Measure type: arithmetic mean. Precision/dispersion type: standard deviation.

TABLE 72

| | | Prior On-demand | | Switch | |
|---|---|---|---|---|---|
| | | Participants | | Participants | |
| Pharmacodynamic Assessment | Category | Mean | Standard | Mean | Standard |
| at Steady State: Minimum | | | Deviation | | Deviation |
| Plasma Concentration During | | | | | |
| the Partial Dosing Interval | | | | | |
| at Steady State (Cmin; ss) | | | | | |
| Based on Factor VIII | | | | | |
| Clotting (FVIII:C) Activity | | | | | |
| Units: hours | | | | | |

| | | |
|---|---|---|
| Number | 9 Participants | 7 Participants |
| Analyzed: | | |

| | | | |
|---|---|---|---|
| 15.8 | 8.6 | 22.9 | 11.3 |

65. Secondary: Factor VIII (FVIII) Clotting Activity.

Description: FVIII clotting activity (FVIII:C) levels was assessed. Measure type: arithmetic mean. Precision/dispersion type: standard deviation.

TABLE 73

| | | Prior On-demand | | Switch | |
|---|---|---|---|---|---|
| | | Participants | | Participants | |
| | Number of | 10 | | 8 | |
| | Participants | | | | |
| | Analyzed: | | | | |
| Factor VIII (FVIII) | Category | Mean | Standard | Mean | Standard |
| clotting activity. | | | Deviation | | Deviation |
| Units: IU/dL | | | | | |

| | | | |
|---|---|---|---|
| At Baseline | Number | 10 Participants | 8 Participants |
| | Analyzed: | | |

| | | | |
|---|---|---|---|
| 6.5 | 10.05 | 6.0 | 7.21 |

TABLE 74
Adverse Events
ALL-CAUSE MORTALITY

| | Prior On-demand | Switch |
|---|---|---|
| | Participants | Participants |
| Total Number Affected | 0 | 0 |
| Total Number At Risk | 13 | 10 |

TABLE 75
Serious Adverse Events

| | Prior On-demand | Switch |
|---|---|---|
| | Participants | Participants |
| Total # Affected by any | 1 | 2 |
| Serious Adverse Event | | |
| Total # at Risk by any | 13 | 10 |
| Serious Adverse Event | | |
| Total # affected by Non | 10 | 6 |
| Serious Adverse Events | | |

Infections and infestations

Urinary tract infection¹

| | | |
|---|---|---|
| Number of participants affected | 0 | 1 |
| Number of events | 0 | 1 |
| Number of participants at risk | 13 | 10 |
| [blank = Total] | | |

Injury, poisoning and procedural complications
Multiple injuries

| | | |
|---|---|---|
| Number of participants affected | 1 | 0 |
| Number of events | 1 | 0 |
| Number of participants at risk | 13 | 10 |
| [blank = Total] | | |

Musculoskeletal and connective
tissue disorders

Rheumatoid arthritis

| | | |
|---|---|---|
| Number of participants affected | 0 | 1 |
| Number of events | 0 | 1 |
| Number of participants at risk | 13 | 10 |
| [blank = Total] | | |

† Events were collected by systematic assessment
¹Term from vocabulary, MedDRA 23.0

TABLE 76
Other Adverse Events
Frequency Threshold for reporting Other Adverse Events: 5%

| | Prior On-demand | Switch |
|---|---|---|
| | Participants | Participants |
| Total # Affected by any | 10 | 6 |
| Other Adverse Event | | |
| Total # at Risk by any | 13 | 10 |
| Other Adverse Event | | |

Cardiac disorders

Supraventricular tachycardia

| | | |
|---|---|---|
| Number of participants affected | 1 | 0 |
| Number of events | 1 | 0 |
| Number of participants at risk | 13 | 10 |
| [blank = Total] | | |

Ventricular extrasystoles

| | | |
|---|---|---|
| Number of participants affected | 1 | 0 |
| Number of events | 1 | 0 |
| Number of participants at risk | 13 | 10 |
| [blank = Total] | | |

Gastrointestinal disorders

Diarrhea

| | | |
|---|---|---|
| Number of participants affected | 0 | 1 |
| Number of events | 0 | 1 |
| Number of participants at risk | 13 | 10 |
| [blank = Total] | | |

Flatulence

| | | |
|---|---|---|
| Number of participants affected | 0 | 1 |
| Number of events | 0 | 1 |
| Number of participants at risk | 13 | 10 |
| [blank Total] | | |

Toothache

| | | |
|---|---|---|
| Number of participants affected | 1 | 0 |
| Number of events | 1 | 0 |
| Number of participants at risk | 13 | 10 |
| [blank = Total] | | |

General disorders and
administration site conditions

Injection site irritation

| | | |
|---|---|---|
| Number of participants affected | 1 | 0 |
| Number of events | 1 | 0 |
| Number of participants at risk | 13 | 10 |
| [blank = Total] | | |

Infections and infestations

Ear infection

| | | |
|---|---|---|
| Number of participants affected | 2 | 0 |
| Number of events | 2 | 0 |
| Number of participants at risk | 13 | 10 |
| [blank = Total] | | |

Gastroenteritis

| | | |
|---|---|---|
| Number of participants affected | 0 | 2 |
| Number of events | 0 | 2 |
| Number of participants at risk | 13 | 10 |
| [blank = Total] | | |

Herpes virus infection

| | | |
|---|---|---|
| Number of participants affected | 1 | 0 |
| Number of events | 1 | 0 |
| Number of participants at risk | 13 | 10 |
| [blank = Total] | | |

Nasopharyngitis

| | | |
|---|---|---|
| Number of participants affected | 1 | 0 |
| Number of events | 1 | 0 |
| Number of participants at risk | 13 | 10 |
| [blank = Total] | | |

Oral herpes

| | | |
|---|---|---|
| Number of participants affected | 0 | 1 |
| Number of events | 0 | 1 |
| Number of participants at risk | 13 | 10 |
| [blank = Total] | | |

Pharyngitis

| | | |
|---|---|---|
| Number of participants affected | 0 | 1 |
| Number of events | 0 | 1 |
| Number of participants at risk | 13 | 10 |
| [blank = Total] | | |

Upper respiratory tract infection

| | | |
|---|---|---|
| Number of participants affected | 0 | 1 |
| Number of events | 0 | 1 |
| Number of participants at risk | 13 | 10 |
| [blank = Total] | | |

Urinary tract infection

| | | |
|---|---|---|
| Number of participants affected | 1 | 0 |
| Number of events | 1 | 0 |
| Number of participants at risk | 13 | 10 |
| [blank = Total] | | |

Injury, poisoning and
procedural complications

Fall

| | | |
|---|---|---|
| Number of participants affected | 1 | 0 |
| Number of events | 1 | 0 |
| Number of participants at risk | 13 | 10 |
| [blank = Total] | | |

Joint injury

| | | |
|---|---|---|
| Number of participants affected | 2 | 0 |
| Number of events | 2 | 0 |
| Number of participants at risk | 13 | 10 |
| [blank = Total] | | |

Investigations

Alanine aminotransferase increased

| | | |
|---|---|---|
| Number of participants affected | 1 | 1 |
| Number of events | 1 | 1 |
| Number of participants at risk | 13 | 10 |
| [blank = Total] | | |

Aspartate aminotransferase increased

| | | |
|---|---|---|
| Number of participants affected | 1 | 0 |
| Number of events | 1 | 0 |
| Number of participants at risk | 13 | 10 |
| [blank = Total] | | |

Metabolism and nutrition disorders

Gout¹

| | | |
|---|---|---|
| Number of participants affected | 1 | 0 |
| Number of events | 1 | 0 |
| Number of participants at risk | 13 | 10 |
| [blank = Total] | | |

Increased appetite

| | | |
|---|---|---|
| Number of participants affected | 0 | 1 |
| Number of events | 0 | 1 |
| Number of participants at risk | 13 | 10 |
| [blank = Total] | | |

Iron deficiency

| | | |
|---|---|---|
| Number of participants affected | 1 | 0 |
| Number of events | 1 | 0 |
| Number of participants at risk | 13 | 10 |
| [blank = Total] | | |

Vitamin B12 deficiency

| | | |
|---|---|---|
| Number of participants affected | 1 | 0 |
| Number of events | 1 | 0 |
| Number of participants at risk | 13 | 10 |
| [blank = Total] | | |

Musculoskeletal and connective
tissue disorders

Arthralgia

| | | |
|---|---|---|
| Number of participants affected | 1 | 2 |
| Number of events | 1 | 2 |
| Number of participants at risk | 13 | 10 |
| [blank = Total] | | |

Neck pain

| | | |
|---|---|---|
| Number of participants affected | 0 | 1 |
| Number of events | 0 | 1 |
| Number of participants at risk | 13 | 10 |
| [blank = Total] | | |

Seronegative arthritis

| | | |
|---|---|---|
| Number of participants affected | 1 | 0 |
| Number of events | 1 | 0 |
| Number of participants at risk | 13 | 10 |
| [blank = Total] | | |

Nervous system disorders

Headache

| | | |
|---|---|---|
| Number of participants affected | 4 | 0 |
| Number of events | 4 | 0 |
| Number of participants at risk | 13 | 10 |
| [blank = Total] | | |

Psychiatric disorders

Sleep disorder

| | | |
|---|---|---|
| Number of participants affected | 1 | 0 |
| Number of events | 1 | 0 |
| Number of participants at risk | 13 | 10 |
| [blank = Total] | | |

Skin and subcutaneous
tissue disorders

Purpura

| | | |
|---|---|---|
| Number of participants affected | 1 | 0 |
| Number of events | 1 | 0 |
| Number of participants at risk | 13 | 10 |
| [blank = Total] | | |

Rash pruritic

| | | |
|---|---|---|
| Number of participants affected | 0 | 1 |
| Number of events | 0 | 1 |
| Number of participants at risk | 13 | 10 |
| [blank = Total] | | |

† Events were collected by systematic assessment
¹Term from vocabulary, MedDRA 23.0

TABLE 77
Population of Trial Subjects

| | Actual number of |
|---|---|
| Country | subjects enrolled |
| Canada | 1 |
| France | 2 |
| Germany | 2 |
| Italy | 3 |
| Netherlands | 1 |
| Russian Federation | 4 |
| Spain | 2 |
| Turkey | 7 |
| United States | 1 |
| Total Worldwide | 23 |
| Total: EEA | 10 |

20 subjects were adults between the age of 18-64 years and 3 subjects were older than 65 years. The total number of subjects was 23.

TABLE 78
Products

| | | Product | Dosage and | Pharmaceutical | Routes of |
|---|---|---|---|---|---|
| Arm | Product Name | Other Name | Administration Details | Forms | Administration |
| Prior | Recombinant | vonicog | Subjects received an | Infusion | Intravenous |
| On- | von | alpha | initial prophylactic dose | | use |
| demand | Willebrand | | of 50 +/− 10 IU/kg, | | |
| Subjects | factor | | intravenous, infusion, | | |
| | (rVWF) | | twice per week for up to | | |
| | | | 12 months. | | |
| Prior | Recombinant | octocog | Subjects received | Infusion | Intravenous |
| On- | Factor VIII | alfa/ | rFVIII (octocog | | use |
| demand | (rFVIII) | ADVATE | alfa/ADVATE), | | |
| Subjects | | | intravenous, infusion for | | |
| | | | treatment of bleeding | | |
| | | | episode. | | |
| Switch | Recombinant | vonicog | Subjects received | Infusion | Intravenous |
| Subjects | von | alpha | rVWF at an initial | | use |
| | Willebrand | | prophylactic dose | | |
| | factor | | of +/−10% | | |
| | (rVWF) | | of weekly pdVWF | | |
| | | | dose received prior to | | |
| | | | this study, intravenous, | | |
| | | | infusion, twice per | | |
| | | | week for up to 12 | | |
| | | | months. | | |
| Switch | Recombinant | octocog | Subjects received | Infusion | Intravenous |
| | Factor VIII | alfa/ | rFVIII (octocog | | use |
| | (rFVIII) | ADVATE | alfa/ADVATE), | | |
| | | | intravenous, infusion for | | |
| | | | treatment of bleeding | | |
| | | | episode. | | |

TABLE 79
Gender and Categorical Characteristics

| | Prior On-demand | Switch | |
|---|---|---|---|
| Reporting Group Values | Subjects | Subjects | Total |
| Overall number of baseline | 13 | 10 | 23 |
| subjects | | | |
| Female | 8 | 3 | 11 |
| Male | 5 | 7 | 12 |

RACE

| | | | |
|---|---|---|---|
| American Indian or Alaska | 0 | 0 | 0 |
| Native | | | |
| Asian | 0 | 0 | 0 |
| Native Hawaiian or Other | 0 | 0 | 0 |
| Pacific Islander | | | |
| Black or African American | 0 | 0 | 0 |
| White | 13 | 9 | 22 |
| More than one race | 0 | 0 | 0 |
| Unknown or Not Reported | 0 | 1 | 1 |

ETHNICITY

| | | | |
|---|---|---|---|
| Hispanic or Latino | 0 | 2 | 2 |
| Not Hispanic or Latino | 13 | 7 | 20 |
| Unknown or Not Reported | 0 | 1 | 1 |

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for prophylactically treating spontaneous bleeding episodes in a subject with severe von Willebrand Disease (VWD) comprising administering to the subject twice-weekly at least one dose of recombinant von Willebrand Factor (rVWF) ranging from at least about 40 IU/kg to about 80 IU/kg, thereby reducing the frequency and/or duration of spontaneous bleeding episodes.

2. The method of claim 1, wherein the at least one dose of rVWF ranges from at least about 50 IU/kg to about 80 IU/kg.

3. The method of claim 1 or 2, wherein the subject has a baseline VWF ristocetin cofactor activity (VWF:RCo) ranging from 20 IU/dL or less, or is diagnosed with Type 1 VWD.

4. The method of claim 1 or 2, wherein the subject is diagnosed with Type 2A, 2B, or 2M VWD.

5. The method of claim 1 or 2, wherein the subject has a VWF:antigen content (VWF:Ag) ranging from 3 IU/dL or greater, or is diagnosed with Type 3 VWD.

6. The method of any one of claims 1 to 5, wherein the subject has been administered prophylactic treatment of plasma-derived VWF (pdVWF) within the past 12 months before initial administration of rVWF.

7. The method of any one of claims 1 to 6, wherein the subject has experienced at least 3 spontaneous bleeding episodes within the past 12 months.

8. The method of any one of claims 1 to 7, wherein the administration occurs every 3 to 4 days.

9. The method of any one of claims 1 to 8, wherein the administration occurs on day 1 and day 5, day 2 and day 6, or day 3 and day 7 of a 7 day period.

10. The method of any one of claims 1 to 9, wherein the administration occurs at least every 24 hours, 36 hours, 48 hours, 72 hours, or 84 hours.

11. The method of any one of claims 1 to 10, wherein the administration occurs at least every 72 hours.

12. The method of any one of claims 1 to 11, further comprising administering to the subject at least one dose of recombinant Factor VIII (rFVIII).

13. The method of claim 12, wherein the administration of the at least one dose of rFVIII is concomitantly or sequentially administered with the at least one dose of rVWF.

14. The method of any one of the preceding claims, wherein the subject resumes the prophylactic treatment after receiving elective surgery or oral surgery.

15. The method of claim 14, wherein if the elective surgery is minor surgery or oral surgery and the subject has FVIII activity (FVIII:C) of at least 0.4 IU/mL or greater, the subject is administered rVWF without rFVIII before surgery.

16. The method of claim 14, wherein if the elective surgery is major surgery and the subject has FVIII activity (FVIII:C) of at least 0.8 IU/mL or greater, the subject is administered rVWF without rFVIII before surgery.

17. The method of any one of claims 1 to 16, wherein prophylactic treatment efficacy is indicated by a reduction of ≥25% in annual bleeding rate (ABR) for spontaneous bleeding episodes during rVWF prophylaxis relative to the pretreatment ABR.

18. The method of any one of claims 1 to 16, wherein prophylactic treatment efficacy is indicated by a reduction of ≥25%, ≥30%, ≥35%, ≥40%, ≥45%, ≥50%, ≥55%, ≥60%, ≥65%, ≥70%, ≥75%, ≥80%, ≥85%, ≥90%, or ≥95% in annual bleeding rate (ABR) for spontaneous bleeding episodes during rVWF prophylaxis relative to the pretreatment ABR.

19. The method of any one of claims 1 to 18, wherein prophylactic treatment efficacy is measured by examining vWF:RCo and/or FVIII activities in samples obtained from the subject before and after prophylactic treatment with rVWF.

20. The method of any one of claims 1 to 19, wherein prophylactic treatment efficacy is measured by examining FVIII, FVIII:C, VWF:RCo, VWF:Ag and/or VWF collagen-binding capacity activities in samples obtained from the subject before and after prophylactic treatment with rVWF.

21. The method of claim 19 or 20, wherein the samples for examining FVIII, FVIII:C, VWF:RCo, VWF:Ag, and/or VWF collagen-binding capacity activities are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF.

22. The method of any one of claims 19 to 21, wherein the samples for examining FVIII, FVIII:C, VWF:RCo, VWF:Ag, and/or VWF collagen-binding capacity activities are obtained 25 to 31 days after prophylactic treatment with rVWF.

23. The method of any one of claims 1 to 22, wherein prophylactic treatment efficacy is determined after or during a bleeding episode, wherein samples for examining FVIII, FVIII:C, VWF:RCo, VWF:Ag, and/or VWF collagen-binding capacity activities are obtained after the bleeding episode, and further wherein the samples are obtained prior to rVWF administration, 2 hours after rVWF administration and then every 12-24 hours until resolution of the bleeding episode.

24. The method of any one of claims 1 to 23, wherein prophylactic treatment efficacy is indicated by an improvement in FVIII, FVIII:C, VWF:RCo, VWF:Ag, and/or VWF collagen-binding capacity activity levels after the prophylactic treatment with rVWF as compared to the levels prior to the prophylactic treatment with rVWF.

25. A method for prophylactically treating spontaneous bleeding episodes in a subject with severe von Willebrand Disease (VWD) comprising administering to the subject a weekly dose of recombinant von Willebrand Factor (rVWF) substantially equivalent to a corresponding weekly dose of plasma-derived VWF (pdVWF) previously administered to said subject, thereby reducing the frequency and/or duration of spontaneous bleeding episodes.

26. The method of claim 25, wherein the weekly dose of rVWF is about 10% less than the corresponding weekly dose of pdVWF.

27. The method of claim 25, wherein the weekly dose of rVWF is about 10% more than the corresponding weekly dose of pdVWF.

28. The method of any one of claims 25 to 27, wherein the weekly dose of rVWF is two individual infusions administered on separate days.

29. The method of any one of claims 25 to 27, wherein the weekly dose of rVWF is three individual infusions administered on separate days.

30. The method of any one of claims 25 to 27, wherein the weekly dose of rVWF is a single infusion.

31. The method of any one of claims 28 to 30, wherein each individual infusion comprises up to 80 IU/kg rVWF.

32. The method of any one of claims 28 to 31, wherein each individual infusion comprises 80 IU/kg rVWF.

33. The method of any one of claims 28 to 31, wherein each individual infusion comprises 50 IU/kg rVWF.

34. The method of any one of claims 25 to 33, wherein the subject has a baseline VWF ristocetin cofactor activity (VWF:RCo) ranging from 20 IU/dL or less, or is diagnosed with Type 1 VWD.

35. The method of any one of claims 25 to 34, wherein the subject is diagnosed with Type 2A, 2B, or 2M VWD.

36. The method of any one of claims 25 to 35, wherein the subject has a VWF:antigen content (VWF:Ag) ranging from 3 IU/dL or greater, or is diagnosed with Type 3 VWD.

37. The method of any one of claims 25 to 36, wherein the subject has received prophylactic treatment of pdVWF for at least 12 months.

38. The method of any one of claims 28 and 31 to 37, wherein the two individual infusions are administered on day 1 and day 5, or day 2 and day 6, or day 3 and day 7 of a 7 day period.

39. The method of any one of claims 27 and 31 to 37, wherein the three individual infusions are administered on day 1, day 3, and day 6 of a 7 day period.

40. The method of any one of claims 25 to 37, wherein the administration occurs at least every 24 hours, 36 hours, 48 hours, 72 hours, or 84 hours.

41. The method of any one of claims 25 to 37, wherein the administration occurs at least every 72 hours.

42. The method of any one of claims 25 to 41, further comprising administering to the subject at least one dose of recombinant Factor VIII (rFVIII).

43. The method of claim 42, wherein the administration of the at least one dose of rFVIII is concomitantly or sequentially administered with the weekly dose of rVWF.

44. The method of any one of claims 25 to 43, wherein the subject resumes the prophylactic treatment after receiving elective surgery or oral surgery.

45. The method of claim 44, wherein if the elective surgery is minor surgery or oral surgery and the subject has a FVIII activity (FVIII:C) of at least 0.4 IU/mL or greater, the subject is administered rVWF without rFVIII prior to surgery.

46. The method of claim 44, wherein if the elective surgery is major surgery and the subject has a FVIII activity (FVIII:C) of at least 0.8 IU/mL or greater, the subject is administered rVWF without rFVIII prior to surgery.

47. The method of any one of claims 25 to 43, wherein prophylactic treatment efficacy is indicated by a reduction of >25% in annual bleeding rate (ABR) for spontaneous bleeding episodes during rVWF prophylaxis relative to the pretreatment ABR.

48. The method of any one of claims 25 to 47, wherein prophylactic treatment efficacy is indicated by a reduction of ≥25%, ≥30%, ≥35%, ≥40%, ≥45%, ≥50%, ≥55%, ≥60%, ≥65%, ≥70%, ≥75%, ≥80%, ≥85%, ≥90%, or ≥95% in annual bleeding rate (ABR) for spontaneous bleeding episodes during rVWF prophylaxis relative to the pretreatment ABR.

49. The method of any one of claims 25 to 48, wherein prophylactic treatment efficacy is measured by examining vWF:RCo and/or FVIII activities in samples obtained from the subject before and after prophylactic treatment with rVWF.

50. The method of any one of claims 25 to 49, wherein prophylactic treatment efficacy is measured by examining FVIII, FVIII:C, VWF:RCo, VWF:Ag, and/or VWF collagen-binding capacity activities in samples obtained from the subject before and after prophylactic treatment with rVWF.

51. The method of claim 49 or 50, wherein the samples for examining FVIII, FVIII:C, VWF:RCo, VWF:Ag, and/or VWF collagen-binding capacity activities are obtained 15 minutes, 30 minutes, 60 minutes, 3 hours, 6 hours, 12 hours, 24 hours, 28 hours, 32 hours, 48 hours, 72 hours, or 96 hours, after prophylactic treatment with rVWF.

52. The method of any one of claims 49 to 51, wherein the samples for examining FVIII, FVIII:C, VWF:RCo, VWF:Ag, and/or VWF collagen-binding capacity activities are obtained 25 to 31 days after prophylactic treatment with rVWF.

53. The method of any one of claims 25 to 52, wherein prophylactic treatment efficacy is determined after or during a bleeding episode, wherein samples for examining FVIII, FVIII:C, VWF:RCo, VWF:Ag, and/or VWF collagen-binding capacity activities are obtained after the bleeding episode, and further wherein the samples are obtained prior to rVWF administration, 2 hours after rVWF administration and then every 12-24 hours until resolution of the bleeding episode.

54. The method of any one of claims 25 to 53, wherein prophylactic treatment efficacy is indicated by an improvement in FVIII, FVIII:C, VWF:RCo, VWF:Ag, and/or VWF collagen-binding capacity activity levels after the prophylactic treatment with rVWF as compared to the levels prior to the prophylactic treatment with rVWF.

55. The method of any one of the preceding claims, wherein spontaneous bleeding comprises any one selected from the group consisting of hemarthrosis, epistaxis, muscle bleeding, oral bleeding, and gastrointestinal bleeding.

56. The method of any one of the preceding claims, wherein the subject is not diagnosed with type 2N VWD or pseudo VWD.
